SEQUENCE LISTING (8 sequences)

SEQ ID NO: 1 - protein, 14 aa, Artificial Sequence
  Description of Artificial Sequence Synthetic peptide
LAPITYPQGLALAK

SEQ ID NO: 2 - protein, 15 aa, Artificial Sequence
  Description of Artificial Sequence Synthetic peptide
KLAPITYPQGLALAK

SEQ ID NO: 3 - protein, 12 aa, Artificial Sequence
  Description of Artificial Sequence Synthetic peptide
NPEQEPIPIVLR

SEQ ID NO: 4 - protein, 18 aa, Artificial Sequence
  Description of Artificial Sequence Synthetic peptide
AFDAESDPSNAPGSGTEK

SEQ ID NO: 5 - protein, 21 aa, Artificial Sequence
  Description of Artificial Sequence Synthetic peptide
GHFPFTHVRLLDQQNPDEDFS

SEQ ID NO: 6 - protein, 15 aa, Artificial Sequence
  Description of Artificial Sequence Synthetic peptide
FPFVAVSIGFAVNKK

SEQ ID NO: 7 - protein, 19 aa, Artificial Sequence
  Description of Artificial Sequence Synthetic peptide
LKEQGQAPITPQQGQALAK

SEQ ID NO: 8 - protein, 27 aa, Artificial Sequence
  Description of Artificial Sequence Synthetic peptide
IREPLVIFCATTGQGDPPDNMKNFWRF

(19) United States
(12) Patent Application Publication
(10) Pub. No.: US 2008/0124310 A1
(43) Pub. Date: May 29, 2008

(54) Bead based receptor biology

(76) Inventors: John G. Marshall, Toronto (CA); Andrzej Jankowski, Milton (CA)

Correspondence Address: MCHALE & SLAVIN, P.A., 2855 PGA BLVD, PALM BEACH GARDENS, FL, 33410, US

(21) Appl. No.: 11/590,967

(22) Filed: Nov. 1, 2006

(51) Int. Cl. A61K 38/43; C12N 5/06; C12Q 1/68; G01N 33/53

(52) U.S. Cl. 424/94.1; 435/375; 435/6; 435/7.2; 514/2

(57) ABSTRACT

A method for capturing activated receptor signaling complexes from live cells, utilizing bead based biology wherein live cells are contacted with ligand coated beads to form bead binding sites and thereby initiating formation of a ligand-receptor complex at said bead binding site; and a process for distinguishing and confirming non-specifically bound proteins from specifically bound receptor complexes by utilization of one or more methods of biochemical or biophysical analysis, thereby providing, in a preferred embodiment, a utilization of confocal microscopy and proteomic mass spectroscopy.

### FIELD OF THE INVENTION

This invention relates to a method for capturing activated receptor signaling complexes from live cells; particularly to a method for utilizing bead based biology wherein live cells are contacted with ligand coated beads to form bead binding sites and thereby initiating formation of a ligand-receptor complex at said bead binding site; and most particularly to a process for distinguishing and confirming non-specifically bound proteins from specifically bound receptor complexes by utilization of one or more methods of biochemical or biophysical analysis, thereby providing, in a preferred embodiment, a utilization of confocal microscopy and proteomic mass spectroscopy.

### BACKGROUND OF THE INVENTION

Ligands presented on microscopic beads to live cells stimulate formation of receptor complexes at or near the surface of the cell. Two of the most powerful technologies applied to biological discoveries are laser confocal microscopy and proteomic identification of proteins by tandem mass spectrometry. Confocal microscopy permits in situ observation of proteins performing their cellular functions including interacting with other proteins to form cellular signaling complexes using cellular protocols and techniques. Proteomic identification permits direct elucidation of the identity of proteins within cellular signaling complexes using biochemical protocols and techniques without the need for secondary immunoglobulin reagents. There exists a need for a new technology capable of directly linking confocal microscopy to proteomic mass spectrometry such that the cellular and biochemical techniques can work together on the identical signaling complex in tandem.

In order to accomplish this, the instant inventors have devised a multi-stage bead-based biology system. In the first stage, microscopic beads coated with appropriate ligand(s) can be used to trigger the formation of signaling complexes on the surface of live otherwise unaltered cells. The beads can be excluded from the remainder of the cellular content and other impurities/contaminants, and collected while still associated with receptors and unknown signal complex proteins which then can be identified by mass spectrometry. In the second validation stage, the same beads can be used to verify the participation of the discovered proteins by confocal microscopy in a quantitative and qualitative manner thus unifying these two powerful technologies.

This bead based biological system provides a solution whereby a ligand is affixed to a bead and the bead is, in turn, used to measure the recruitment of members of the signaling pathway by microscopy and to capture the associated proteins by mass spectrometry. The bead thus serves as the link between cell biology and mass spectrometry with a self-validation step built into the process.

### DESCRIPTION OF THE PRIOR ART

Various technologies have heretofore been utilized to assist in the analysis of cell biology and protein-protein interactions.

### Beads without Ligands after Internalization by the Cell

The technologies of mass spectrometry and confocal microscopy, have previously been combined using beads without ligands, and used separately, to examine the internalized phagosome, a membrane bound organelle within phagocytic cells, 30 minutes after engulfment. From these experiments it has been taught that proteins associated with the endoplasmic reticulum membrane and proteins such as GRP78 play the main role in the machinery that internalizes latex beads with no added ligand.

### Surface Proteins Using Biotin/Streptavidin

Labeling of the surface with biotin and collecting the surface proteins using streptavidin affinity chromatography has been demonstrated to collect cell surface proteins in an un-biased manner. However this method is not specific or ideal for isolating activated receptor complexes.

### SELDI (Surface-Enhanced Laser Desorption Ionization)

In contrast to conventional chromatography that uses 3 dimensional beads or supports made of carbohydrates or polymers or ceramics or silica or ceramics or others it is possible to perform chromatographic separations on 2 dimensional surfaces such as SELDI chips. Protein-protein interactions have been achieved on SELDI Chips. SELDI chips are chromatographic surfaces, including normal phase and others that serve directly as sample introduction surfaces in MADLI mass spectrometry. However it is possible to perform chromatography on two dimensional surfaces that do not serve directly as the sample introduction surface for a mass spectrometer but rather serve to capture analytes that are eluted off the 2 dimensional surface for subsequent analysis.

### TAP Tagging

There are several technologies for capturing interacting protein-protein complexes. The used of traditional one step affinity chromatography may not always lead to sufficient quantity or purity of proteins that interact with receptor complexes to identify these proteins by mass spectrometry. The use of tandem affinity purification on 3 dimensional beads may solve this problem in some cases. However this method marred with a high background due to the large non-specific sample capacity and low specific ligand density on 3 dimensional beads. Three dimensional beads contain pores which permit a very large non-specific surface area that may not be coated in specific ligands.

These prior art methods failed to provide the researcher with a methodology capable of harvesting, identifying and validating all participants of signaling complex that form on the surface of a live cell in culture in unaltered or altered (small molecule/drug treated) form.

The prior art failed to teach or suggest the instant technology which 1) enables one to place any ligand on a nano to micro-meter bead and to present the beads to the surface receptors of a live cell in culture, whereby the receptors for the ligand under study bind to the beads and activate the associated signaling pathway that will collect at the site of contact of the bead with the cell surface; 2) in parallel fashion, provides a methodology wherein the as yet unknown proteins that accumulate at the site of the activated receptors can be mined by collecting the ligand-coated beads away from the rest of the cellular content and then identifying the proteins recruited to the beads using LC-MS protein analysis; 3) provides a means whereby the interactions of proteins that are hypothesized to participate in the pathway could then be directly visualized by fusion of their coding sequences with sequences encoding fluorescent proteins followed by transfection of the constructs into cell in culture or by antibody staining, such that the role of the newly identified proteins in the signaling event will be subsequently confirmed using drugs or by knocking out the protein at the cellular level using expression of mutant constructs or sRNAi or knock-out cell lines; and 4) ultimately visualizing the effect on cellular and protein functions by the use of confocal microscopy analysis of the interaction of the ligand coated beads with the cells.

### SUMMARY OF THE INVENTION

In contrast with the prior art, the instant invention compared presenting beads with the specific ligand bound to the activated surface receptor complexes of live cells versus similar control beads incubated with cellular homogenates. A computer or manual inspection or isotopic or isobaric tagging was used to compare the receptor proteins to the control bead proteins and thus subtract the non-specific background proteins that contaminate the beads during isolation and that do not accumulate at the activated receptor: It is shown herein that subsequent cell staining of expression of GFP constructs confirms that the proteins specifically observed in the ligand-receptor complex by mass spectrometry were observed to subsequently accumulate at the same types of ligand coated beads using confocal microscopy or biochemical methods.

Furthermore, it is shown herein that this technology will work differentially with a variety of ligands and thus may form the basis for a general method to detect and elucidate important receptor associated drug targets. The bead system can be used to verify the results of the mass spectrometer and detect proteins that accumulate above background at the site of the ligand coated bead using antibodies and fluorescent proteins. The bead system can subsequently be used with drugs, overexpression of wild type form, mutants or silencing RNA to prove the importance of the protein in receptor function. Finally the same bead system can be used with reporter constructs to monitor and characterize the capacity of drugs or therapeutic agents to effect receptor function, cellular response or metabolism. In contrast to the prior art, instead of only detecting apparent cellular contaminants, the present invention detected the proteins associated with the known signal pathway proteins of the Fc receptor and new novel drug targets not previously detected have been verified. In addition, protein-ligand interactions of proteins discovered by the ligand bead method may be performed on 2 dimensional surfaces prior to analysis by mass spectrometry.

The instantly disclosed bead-based biology technology enables a researcher to harvest, identify and validate all participants of signaling complex that form on the surface of a live cell in culture in unaltered or altered (small molecule/drug treated) form. Simply put, this technology enables one to place any ligand, for example immunoglobulin G (IgG) or OX LDL, on a nano to micro-meter bead and to present the beads to the surface receptors of a live cell in culture. The receptors for the ligand under study bind to the beads and activate the associated signaling pathway that will collect at the site of contact of the bead with the cell surface.

In parallel, the as yet unknown proteins that accumulate at the site of the activated receptors can be mined by collecting the ligand-coated beads away from rest of the cellular content and then identifying the proteins recruited to the beads using LC-MS protein analysis. The interactions of proteins that are hypothesized to participate in the pathway could then be directly visualized by fusion of their coding sequences with sequences encoding fluorescent proteins followed by transfection of the constructs into cell in culture or by antibody staining. The role of the newly identified proteins in the signaling event will then be subsequently confirmed using drugs or by knocking out the protein at the cellular level using expression of mutant constructs or sRNAi or knock-out cell lines and visualizing the effect on cellular and protein functions by the use of confocal microscopy analysis of the interaction of the ligand coated beads with the cells.

Accordingly, it is a primary objective of the instant invention to provide a comprehensive bead based receptor biology method which provides the researcher with a methodology capable of harvesting, identifying and validating all participants of signaling complex that form on the surface of a live cell in culture in unaltered or altered (small molecule/drug treated) form.

It is a further objective of the instant invention to provide a technology which enables one to place any ligand on a nano to micro-meter bead and to present the beads to the surface receptors of a live cell in culture, whereby the receptors for the ligand under study bind to the beads and activate the associated signaling pathway that will collect at the site of contact of the bead with the cell surface.

It is yet another objective of the instant invention to provide a methodology wherein the as yet unknown proteins that accumulate at the site of the activated receptors can be mined by collecting the ligand-coated beads away from the rest of the cellular content and then identifying the proteins recruited to the beads using LC-MS protein analysis.

It is a still further objective of the invention to provide a means whereby the interactions of proteins that are hypothesized to participate in the pathway could then be directly visualized by fusion of their coding sequences with sequences encoding fluorescent proteins followed by transfection of the constructs into cell in culture or by antibody staining, such that the role of the newly identified proteins in the signaling event will then be subsequently confirmed using drugs or by modifying the protein at the cellular level using expression of mutant constructs or sRNAi or knock-out cell lines.

It is yet an additional objective of the instant invention to provide a means for visualizing the effect on cellular and protein functions by the use of confocal microscopy analysis of the interaction of the ligand coated beads with the cells.

Other objects and advantages of this invention will become apparent from the following description taken in conjunction with any accompanying drawings wherein are set forth, by way of illustration and example, certain embodiments of this invention. Any drawings contained herein constitute a part of this specification and include exemplary embodiments of the present invention and illustrate various objects and features thereof.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. Illustrates the formation of a Ligand-Receptor Complex at or near the surface of the cell;

FIG. 2. Illustrates the combination of confocal microscopy and mass spectrometry allows discovery and validation of proteins associated with the ligand-receptor complex at or near the cell surface;

FIG. 3. Shows a strategy for capturing a patch of membrane containing an activated and assembled ligand-receptor complex near or on the cell surface;

FIG. 4. Depicts the identification and verification of presence of protein Actin in the signaling complex near the cell surface. Left panel, Detection of Peptide corresponding to Actin by LC-MS/MS analysis of purified signaling complex/phagosome. Right panels, Verification of Actin presence at the signaling complex using confocal microscopy after binding identical ligand coated beads;

FIG. 5. Panel A represents Internalization of phagosomes/naked beads (prior art), vs. Panel B, cell surface assembly of ligand-receptor complex (present invention);

FIG. 6. Panel A, illustrates the Prior Art, No Ligand on bead show phagosome within the cell; Panel B, Present Invention, illustrates Ligand-receptor complex of any kind on or near the cell surface or within the cell;

FIG. 7. Illustrates the use of confocal microscopy, biochemical and immunological methods to differentiate between non-specific high abundance proteins and those that form signaling complexes during binding of ligand coated beads to the cell surface;

FIG. 8. Illustrates the failure of the ER associated proteins to associate with the developing phagosome or pseudopods. Note that there is no ring of greater intensity staining around the particle during engulfment;

FIG. 9. Shows the positive and negative controls and the work flow of isolating, identifying, confirming and validating target proteins;

FIG. 10. Depicts a molecular model of the signaling network that controls engulfment of particles presenting the Fc receptor ligand IgG. This model has been developed using cytogenetic and genetic mutation studies in mammalian and other model systems, but has not been confirmed by mass spectrometry;

FIG. 11. Shows a model of cell surface receptor facilitating modified lipid particle engulfment to generate foam cells which form the core of atherosclerotic plaques;

FIG. 12. Top, illustrates MS/MS of Fc gamma RIIIA. Bottom, illustrates microscopic image of Fc receptor accumulating at the site of ligand coated bead binding (arrow);

FIG. 13. Top, illustrates MS/MS of Lyn receptor kinase. Bottom, illustrates microscopic images, endogenous receptor Lyn (red) and transiently expressed Lyn-GFP (green) accumulate at the site of ligand coated bead binding (arrow);

FIG. 14. Illustrates a Fluorescence Recovery After Photobleaching (FRAP) assay that demonstrates immobilization of the Src class kinase Lyn within the activated receptor complex upon binding of ligand coated bead;

FIG. 15. Top, illustrates MS/MS of Syk kinase. Bottom, illustrates microscopic image of a transiently expressed Syk-GFP (green). Syk-GFP accumulates at the site of ligand coated bead binding (arrow);

FIG. 16. Top, illustrates MS/MS of Phospholipase C beta 1, Bottom, illustrates microscopic image of a transiently expressed PLC PH-GFP domain (green). PLC PH-GFP accumulates at the site of ligand coated bead binding (arrow);

FIG. 17. Top, illustrates MS/MS of p110 isoform class 1 alpha of PI3K. Bottom left, illustrates microscopic image of a transient PIP3 production by PI3K as measured through the PIP3 binding PH domain of AKT fused to GFP. Bottom right, illustrates expressed p85 subunit of class I PI3K p110 alpha domain (green) localizes to the site of the ligand coated bead interaction with cell surface receptor;

FIG. 18. Top: illustrates MS/MS spectra for a 2+ peptide LAPITYPQGLALAK that correlates with Rac1 isolated with IgG coated magnetic beads binding to the cell surface of human neutrophils. Bottom: illustrates (red) Localization of endogenous Rac1 with anti-Rac1 antibodies in RAW macrophages; (green) Localization of GFP Rac1 expressed in RAW macrophages. Note that Rac1 clearly localizes to the plasma membrane that first engulfs the particle;

FIG. 19. Top: illustrates MS/MS spectra showing a 2+ peptide KLAPITYPQGLALAK correlating with RAC2 isolated with IgG coated magnetic beads binding to the cell surface of human neutrophils. Bottom LEFT: illustrates (red), detection of endogenous Rac2 with anti Rac2 antibodies; Bottom GREEN, illustrates localization of K-RAS C-terminal geranylation sequence fused to GFP in RAW macrophages. Note that the Ras superfamily member localizes with the plasma membrane that first binds the particle;

FIG. 20. Top: illustrates MS/MS spectra correlating to the 2+ peptide NPEQEPIPIVLR of the CDC42 GTPase activating protein isolated from IgG magnetic beads binding to the cell surface of human neutrophils. Bottom: illustrates expression of CDC42 GFP in RAW macrophages and accumulation at the site of ligand coated bead interaction with its receptors at the cell surface;

FIG. 21. Top: illustrates MS/MS spectra showing a 2+ peptide correlating with Dock2 isolated from IgG coated magnetic beads bound to cell surface of human neutrophils. Bottom: (red), illustrates detection of endogenous Dock2 with anti Dock2 antibodies; Note that the Rac regulator Dock2 localizes with the plasma membrane where ligand coated particle binds;

FIG. 22. Top: illustrates MS/MS spectra correlating with the 2+ peptide AFDAESDPSNAPGSGTEK from ELMO2 isolated using IgG coated beads binding to human neutrophils. Bottom: illustrates Localization of ELMO2 GFP expressed in RAW macrophages. Note that ELMO2 localized with the membrane that first binds ligand coated particle;

FIG. 23. Top: illustrates MS/MS spectra correlating with a 3+ peptide GHFPFTHVRLLDQQNPDEDFS from the proto oncogene C-CRK1 (P38 adaptor molecule) isolated with IgG coated magnetic beads bound to cell surface of human neutrophils. Bottom: (red), illustrates detection of endogenous Crk1 with anti Crk1 antibodies; (green) localization of Crk1-GFP fused to GFP in RAW macrophages. In both cases, Crk1 accumulates at the site of ligand coated bead interaction with its receptors at the cell surface of RAW macrophage cells;

FIG. 24. Top: illustrates MS/MS spectra correlating with the 2+ peptide FPFVAVSIGFAVNKK from the lipid 1 or 4 monophosphatase bearing similarity to SHIP-1 isolated using IgG coated beads binding to the cell surface of human neutrophils. Bottom: illustrates Localization of endogenous SHIP-1 expressed in RAW macrophages. Note that SHIP-1 was localized with the membrane that first binds ligand coated particle;

FIG. 25. Top: illustrates Method for quantifying particle uptake using phagocytic receptors assay, Bottom: illustrates Method for measuring effect of transfection or delivery of nucleic acids on particle engulfment and accumulation using a single cell multi label confocal microscope assay;

FIG. 26. Illustrates use of PiP2 binding domains to screen atherosclerosis drugs in macrophages;

FIG. 27. Illustrates use of PiP3 binding PH domains to screen atherosclerosis drugs in macrophages;

FIG. 28. Illustrates use of DAG binding domains to screen for atherosclerosis drugs in macrophages;

FIG. 29. Illustrates monitoring of multiple metabolites or second messengers in series or parallel;

FIG. 30. A, illustrates that modification of lipids results in ligand ox-LDL that binds to cell surface receptors and causes foam cell formation. B, illustrates comparison of modified lipid and IgG uptake when bound to nano- and micro-particles;

FIG. 31. Top, Photographs show control and drug treated (cytochalasin D, wortmannin) cells expressing AKT PH GFP fusion domain as a way to observe PIP3 metabolism at the site of ligand coated particle binding to cell surface. Bottom, Graph indicating quantitative measure of PIP3 generation at the site of ligand coated bead interaction with the cell surface;

FIG. 32. Illustrates phagocytic receptor assay use as a quantitative measure of small molecule PP2 inhibition of Src proteins. Note, Src proteins have been instantly discovered by MS and confirmed by CF as shown in FIG. 13 (discovery) and FIG. 25 (screening phagocytic receptor assay);

FIG. 33 A—depicts kinetics of PIP3 loss with wortmannin versus LY294002 using fluorescent protein domains;

FIG. 33. B—depicts screening of silencing RNA effect on foam cell formation, silencing RNA designed against PI3K class 1 alpha causes reduction in number of particles accumulating within the macrophage cells (red cells labeled with arrows) when compared to cells that did not get transfected with silencing RNA (arrows on DIC/Red left panels);

FIG. 34. Top Left, illustrates MS/MS spectra showing ions for the 2+ peptide LKEQGQAPITPQQGQALAK, 2007.3 correlating to RhoG. Top Right, illustrates Expression of RhoG GFP in RAW macrophages. Note that RhoG localizes to the membrane that binds ligand coated particle. Bottom Left, illustrates Expression of dominant negative RhoG (green) in RAW macrophages. Note that the cell expression DN RhoG has no blue (engulfed) particles;

FIG. 35. Top and Middle, Quantitative examination of mutant nucleic acid effect using phagocytic receptor assay. Bottom, Photographs of mutant nucleic acid effect on ligand coated particle uptake. Most dramatic effect observed in middle (p115_{—}242 to 912) and right photographs (RhoA_G14V) where cells expressing mutant enzyme (light blue) accumulate reduced number of phagocytic ligand coated particles (red for ligand coated particle);

FIG. 36. Illustrates use of phagocytic receptor assay to examine the effects of silencing RNA;

FIG. 37. Top, illustrates MS/MS of p115 RhoGEF. Bottom, illustrates microscopic image of a cell transiently expressing p115 RhoGEF (green). P115 RhoGEF accumulates at the site of ligand coated bead binding (arrow);

FIG. 38. Photographs, Time course of modified lipid LDL uptake by macrophage over 60 minute time period. Graph, Quantitation of observed modified lipid uptake. Table, statin has no effect on fluid phase oxLDL, but it does inhibit particle bound oxLDL accumulation by RAW 264.7 leukocytes;

FIG. 39. Illustrates effect of inhibiting PLD Pathway on Fc mediated phagocytosis using indicated drugs. The control accumulate most ligand coated particles (red). The yellow indicates that ethanol, propranolol and HELSS prevent particle accumulation;

FIG. 40. PKC/C2-GFP Domain measures DAG production at the site of particle engulfment. Penetration and efficacy of Propranolol, HELSS and Ethanol (ETOH) to prevent DAG production is demonstrated;

FIG. 41. Illustrates the use of PKC/C2-GFP Domain to measure DAG production at the site of particle engulfment and to measure the penetrance and efficacy of a potential atherosclerosis drug;

FIG. 42. Illustrates measurement of drug specificity. Akt/PH-GFP domain measures PIP3 production at the site of particle engulfment. The fluorescent signal in the presence of propranolol, HELSS and EtOH indicate that these drugs have no side effect on the PI3K pathway to PIP3;

FIG. 43. Illustrates measurement of drug specificity. The PLC-delta PH domain measures PIP2 catalysis by PLC at the base of the engulfed particle. Note that neither EtOH, HELSS or propranolol interfere with the catalytic action of PLC.

FIG. 44. Illustrates measurement of drug specificity, demonstrates that the inhibitory effect of HELSS on DAG production as measured by the PKC/C2-GFP is not due to an effect on iPLA2. Neither MAFP nor AACOCF3 prevent DAG production in contrast to the PAP-1 inhibitor HELSS;

FIG. 45. Illustrates that PLD pathway inhibitors prevent particle engulfment and the effect is reversed by DiC8;

FIG. 46. Illustrates that propranolol, but not control beta blockers, prevents particle engulfment/foam cell formation;

FIG. 47. Illustrates that PLD pathway inhibitors prevent oxidative burst;

FIG. 48. Illustrates that DiC8 partially recovers inhibition of oxidative burst (A). However arachidonic acid cannot recover inhibition of intact (B) or permeabilized cells (C);

FIG. 49. Illustrates that the PAP-1 inhibitor HELSS but not the iPLA2 inhibitors MAFP Or AACOCF3 inhibit the oxidative burst;

FIG. 50. Illustrates that Propranolol, but not control beta blockers, prevents fMLP inducedoxidative burst;

FIG. 51. Illustrates the effect of statins on engulfment of particles. Statins prevent engulfment (no red) and particles are stranded outside (yellow). Control and Cholesterol Scavenger mβcd (methyl-beta cyclodextrin) still engulf particles (red);

FIG. 52. Illustrates that removal of cellular cholesterol using mβcd has no effect on signaling due to IgG coated bead binding at the cell surface. Top, Filipin staining shows removal of cholesterol at the site of IgG coated particles in mβcd treated RAW cells as compared to untreated control; Bottom, mβcd has no effect on particle uptake and PIP3 accumulation (shown using AKT/PH-GFP domain) at sites of IgG coated particles binding to cell surface.

FIG. 53. Illustrates quantification of the effect of cholesterol lowering drugs on leukocyte macrophage mediated model of foam cell formation;

FIG. 54. Membrane proteins from RAW macrophages cell treated with lovastatin;

FIG. 55 Matrix proteins from RAW macrophages cell treated with lovastatin;

FIG. 56. Secreted proteins from RAW macrophages cell treated with lovastatin;

FIG. 57. Cytosol proteins from RAW macrophages cell treated with lovastatin;

FIG. 58 Illustrates the effect of statins on the surface expression of TSP-1;

FIG. 59. Quantification of inhibitory effect of anti-Thrombospondin-1 antibody on particle engulfment/foam cell formation;

FIG. 60. Illustrates the use of ligand covered beads to demonstrate protein-protein interaction of Actin and HS1;

FIG. 61. Depicts the use of 2D surface to characterize protein-protein interactions of HS1 by mass spectrometry;

FIG. 62. Illustrates the use of RAW macrophages to screen the function of ion channels by studying calcium dependant processes;

FIG. 63. Demonstrates the use of AKT-PH GFP domain to study ionic signaling at the site of ligand coated bead interaction with the cell surface. The effect of free extracellular calcium on PIP3 signaling at the site of ligand coated beads is shown;

FIG. 64. Illustrates the effect of intracellular calcium on the mobility of the SRC class proteins LYN's N terminus fused to GFP;

FIG. 65. Western Blot of RAW Cell Matrix fraction with Phosphotyrosine antibody. Illustrates the kinetics of protein phosphorylation in cell matrix fraction in response to ALF4 or peroxy NaVO4;

FIG. 66. Western blot of cytosol fractions with phosphotyrosine. Illustrates the kinetics of protein phosphorylation in cytosol fraction in response to ALF4 or peroxy NaVO4;

FIG. 67. Cytosol fractions of RAW Cell (7% Tricine Gel), illustrates the kinetics of protein phosphorylation in cytosol fraction (Tris gel shown) in response to ALF4 or peroxy NaVO4;

FIG. 68. Western blot of RAW cell membrane fraction with phosphotyrosine antibody; illustrates the kinetics of protein phosphorylation in a cell membrane fraction in response to ALF4 or peroxy NaVO4;

FIG. 69 A,B, and C. Illustrates J774, CHO cells expressing the Fc receptor and RAW 264.7 leukocytes binding IgG and oxLDL coated 2 um beads at the cell surface. Associated Actin (green) and phospho-Tyrosine accumulation at the vicinity of ligand-coated and receptor associated complex formation is shown, FIG. 69. C, Chinese ovarian hamster cancer cells (CHO cells) express the GFP fusion of Fcg 2A receptor. No binding shows homogeneous receptor distribution. Binding of IgG coated 2 um particles stimulate receptor complex;

FIG. 70. Mascot search results; isotopically labeled peptide belonging to NADPH oxidase is present only in the fraction collected from a signaling complex at the cell surface (labeled with the ICPL light +233.27) reagent and not control (expected label +239.22);

FIG. 71. ITRAQ isobarically labeled 116 control and 117 labeled IgG coated beads pulled from the cell membrane; A, Left panel shows MS/MS of protein PAK2 known and Right panel shows quantification, where it is only observed in the bead coated with IgG ligand when bound to cell surface and not in the control, B, Left panel shows MS/MS of RNA-binding region RNP-1 (RNA recognition motif), Right panel confirms that it is localized at 10× higher concentration in control non-specifically bound fraction than at the 117 labeled IgG ligand coated bead bound to the cell surface.

### DEFINITIONS

In accordance with this disclosure, the following terms will be understood to be defined as follows:

“Activated receptor signaling complexes” refers to all biopolymers along the pathway which moves signals from the ligand via at least one receptor to the sites of their effect within the cell, including the receptor, its directly bound proteins, the surrounding membrane and cytoskeleton, and indirectly bound proteins separated from the receptor in space or time.

“Bead binding site” refers to the location on the surface of the cell where the ligands on the bead have engaged cell surface receptors.

“Ligand” or “Receptor Ligand” refers to a biopolymer or drug which can specifically and mutually bind to a receptor, including albeit not limited to any LDL bound proteins, lipids or derivatives thereof.

“Control Bead” refers to a bead which non-specifically binds biopolymers without the interaction of ligands or receptors yielding a non-specifically bound control complex.

“Non-specifically Bound Control Complex” refers to biopolymers which bind to a control bead.

“Biopolymer” refers to discrete or complexed proteins, carbohydrates, lipids, nucleic acids and combinations thereof.

“Drug” refers to any small molecule compound, e.g. statins, propranolol; or biologically derived compound, e.g. silencing RNA, IgG, a dominant negative construct, an anti-sense DNA, an antibody, morphilinos or the like, effective to alter the natural functioning of a cell biopolymer.

“Cell Biopolymer Function Modulating Material” refers to a drug, a genetic knockout, or any naturally occurring or modified plant or fungal extract, effective to alter the natural functioning of a cell biopolymer.

“Bead” is understood to include any substrate, whether homogeneous or heterogeneous, capable of binding with or to a receptor or group of receptors. Beads may be solid or porous. Beads may be spherical or of an irregular shape or fibrous or square or a flat plane or of another shape. The beads may be of a microscopic, sub-microscopic or macroscopic dimension. A surface of glass or plastic such as a 96 well dish or other 2 dimensional surfaces may be used. The beads may be composed of hydrophobic material or hydrophilic material and may be made of carbohydrates, alginates, gelatins, synthetic or natural polymers, or silicates or any combination thereof. The beads may be derivatized to include one or more chemical moieties including, albeit not limited to, amines, carboxylates, biotin-streptavidin, silanol, polylysine, n-hydroxy succinimide (NHS), n-hydroxysulfosuccinimide, or other silicon based chemistries with or without spacer arms. The beads may be modified by the covalent or non-covalent addition of biopolymers. Functionally, a bead is understood to refer to any micro or nano sized particles useful for the attachment thereto of receptor ligands, wherein said ligand bound micro or nano particles may bind to live cells to engage the receptor and trigger the assembly and recruitment of drug targets to the receptor. Beads without at least one specific receptor ligand may serve as a negative control.

“Modified Beads” or “Modified Particles” are understood to mean beads or particles which have been rendered competent to bind cellular receptors including albeit not limited to phagocytic receptors and activate responses from cells, including albeit not limited to macrophage foam cell precursors and foam cells resultant therefrom, producing physiologically significant outcomes including, albeit not limited to, engulfment or phagocytosis.

“Macrophage Foam Cell Precursors” are understood to include any cultured leukocytes such as macrophages which serve as a model of foam cells in atherosclerotic plaques, including, albeit not limited to RAW macrophages, J774 macrophages and U937 macrophages. Leukocyte and macrophage include, but are not limited to all white blood cells, including macrophages, monocytes, dendritic cells, neutrophils and other white blood cells.

“Receptor Pathway Function” refers to determining particle internalization, or determining changes in accumulation of proteins, or determining changes in accumulation of metabolites, or determining changes in ionic concentrations at or near the bead binding site or activated receptor signaling complex compared to distal locations in the cell or compared to cells without activated receptor signaling complexes.

In accordance with the instant disclosure, the phrase “in conjunction” is understood to mean the carrying out of disparate steps in a process simultaneously, one before the other, or one after the other, the choice of which is judiciously selected in order to insure accumulation of sufficient protein or protein domain in an amount effective to efficiently conduct a required assay step.

### DETAILED DESCRIPTION OF THE INVENTION

Cells are bound by membranes composed of a lipid bilayer. Receptor proteins are proteins on the surface of cells. Receptors may exist on the outer surface of the cells and may or may not extend through the membrane from the outer surface, through the lipid bi-layers and extend within the cytoplasm of the cell. Receptors are proteins or protein, carbohydrate and lipid complexes on the surface of cells. Receptor complexes sense information from the external environment of the cell. The receptor protein and other proteins that move information are signaling proteins. Receptor complexes and their associated proteins are drug targets that are a key focus of pharmacological research. It is very difficult to isolate and identify the proteins associated with receptor complexes. Most methods for identifying the proteins in receptor-associated pathways utilize protein-protein interactions in vitro between the receptor and associated proteins and their binding partners based on the affinity of their binding in solution or genetic or cytogenetic methods relying on observations from mutations. In live cells, receptor complexes are not only based on the interaction between the receptor proteins or other signaling proteins but also may require the organizing structure provided by the membrane and the cytoskeleton. Receptors complexes may be very large composed of hundreds of protein, lipid, carbohydrate and other compounds and may require the energy and ordered structure of the living cell in order to completely assemble. To date there is no method for isolating receptor complexes from live cells and identifying the drug targets within the complex and validating the drug targets within the complex by independent biochemical or biophysical means.

The major problem with isolating intact signaling complexes from cellular extracts is that the homogenization of the cell with mechanical force or detergents randomized the arrangement of proteins and makes re-assembly of the entire signaling complex in vitro difficult to achieve. What is needed is a method that does not require the disruption of the cell and disassembly of the membrane and cytoskeletal protein scaffolds, that may potentially help hold the signaling complex together into one functional unit prior to isolation of receptor complexes from live cells. Also, it may be preferable if the nucleating receptor protein that acts as the center of the complex was binding its ligand and therefore in an active conformation. The object of the present invention is to effect the capture and identification of activated signaling complex on the cell surface and its associated protein complex drug target(s) by mass spectrometry and verify that the identified proteins are functionally associated with the receptor using confocal microscopy or other biochemical assays by a simple and rapid method.

The approach is to put the activating ligand of the signal receptor complex on a bead and allow the bead to interact with the cell of interest. The activation of the signaling complex may be measured in the cells by observing known signaling proteins translocating to the bead or by measurement of the metabolic products of the signaling pathway with a confocal microscope (or by some other measurement) at the ligand-coated bead. Once the time required for the beads to activate (or in-activate) the signaling complex upon introduction of the ligand-bead has been determined the beads may be collected. Collection of the beads by rapidly removing them from the surface of the live cells with mechanical energy serves to pull the bead away from the cell with such force as to pull off a patch of the associated membrane that contains the activated receptor and its associated effector proteins as well as the membrane and cytoskeleton that presumably surround the activated complex and hold the functioning complex together. The mechanical force may be supplied by a powerful magnet if para-magnetic beads are used or just by vigorous shaking of the cells' vessel or by disruption in detergents, homogenization, sonication, the use of a French press, the combination or other methods.

The collected beads remain bound to the area of the membrane wherein the activated receptor complex resides. The proteins associated with the ligand-bead and the proteins associated with the bead without the ligand or with an irrelevant ligand such as BSA as the controls are identified. Thus affixing the receptor ligand to the bead may permit the subsequent recovery of large molecular mass protein signaling complexes bound to the bead with the activated receptor pathway attached. The proteins associated with the signaling system bound to the beads can be identified by enzyme activity, or immunological methods or by mass spectrometry or other biochemical means.

The beads may be extracted and the proteins separated by liquid chromatography or electrophoresis followed by mass spectrometry. The beads themselves serve as the chromatographic resin and the proteins within the attached signaling complex may be released or eluted from the beads by their differential solubility in salts, chaeotropic agents, chelating agents, variations in pH or any other protein solubilizing reagents. Hydrophobic or membrane proteins or other proteins that remain on the beads after extraction of soluble proteins may be extracted for analysis by ionic or non-ionic detergents or other membrane solubilizing agents. Alternatively the insoluble membrane proteins may be directly digested to peptides with proteases in the presence of organic solvents such as methanol, ethanol, acetonitrile or other organic solvents.

The protein or peptide extracts may then be further purified by electrophoresis or chromatography, or 2D electrophoresis, capillary electrophoresis or multi-dimensional liquid chromatography of the proteins and/or multi-dimensional liquid chromatography of the peptides derived from proteolytic digest of the captured and purified proteins. The peptides resulting from the electrophoretically or chromatographically or otherwise separated proteins, or the proteins themselves, can then be identified by mass spectrometry or Edman degradation or by biochemical tests.

The mass spectrometry may be single MS or tandem MS/MS or multiple MS fragmentation performed on a MALDI-TOF, MALDI-Qq-TOF, circular ion trap, tubular ion trap, FTMS or other instruments (mass spectrometry based proteomics).

The results of the mass spectrometry can be scrutinized to compare the proteins identified from beads both with and without the activating ligand or with an irrelevant ligand or with beads that have been blocked, i.e. coated, with molecules to prevent specific or non-specific interactions. The proteins associated with the activating ligand coated beads may be compared to the proteins associated with control beads without the activating ligand or coated with an irrelevant ligand or with a blocking agent. The analysis may be performed manually or with a computer program to indicate which proteins accumulate differentially on the activating ligand beads. Subsequently, the same ligand-coated bead may be used to confirm the members of the activated receptor pathway identified by mass spectrometry by visualizing that these identified proteins play a role in the signaling pathway under consideration by high-resolution confocal microscopy.

The proteins identified by mass spectrometry associated with or differentially present on the captured bead may be confirmed by visualization at the site where the ligand coated bead contacts the cell using similar, or differently sized beads, with a microscope, by labeling the molecule of interest and measuring its recruitment to the site of the activating ligand coated bead. The proteins identified by proteomic analysis of the beads, or others means, may be labeled using immuno-fluorescence or GFP fusion or luciferase or any light emitting proteins, or dyes, or enzyme activities or light emitting proteins, dyes or enzyme activities fused to antibodies or proteins or proteins binding domains or peptides or aptamers or other molecular probes.

The microscope, or other means, can be used to confirm that the novel proteins identified by mass spectrometry play a role in the signaling complex by several means. The ligand coated beads can be introduced to the cells and the activation of the signaling pathway can be observed by the translocation of known signaling proteins to the activating beads or by the production of the metabolic products at the site of the activating bead. The entry of newly discovered protein members into the signaling complex can be observed directly by quantification of the microscopic image or indirectly by Fluorescence Recovery After Photo-bleaching (FRAP) of the implicated new molecules or by Fluorescence Resonance Energy Transfer (FRET) with known signaling molecules or fluorescence correlation analysis or measuring the rate of fluorescence decay of the molecule.

The role of the newly discovered proteins in the signaling pathway can also be confirmed by modifications such as RNA silencing, genetic knockouts and overexpressions of wild type and dominant negative constructs, anti-sense DNA, antibodies, drugs, natural products, small molecules or other methods that alter (inhibit or enhance) the function of the newly discovered proteins or protein iso-forms and observing its effects on the operation of the signaling complex. The effect of these interventions on the function of the signaling complex can be observed by the translocation of known signaling proteins to the activating beads or by the production of the metabolic products at the site of the activating bead or by some other measure of the function of the activated receptor including protein phosphorylation, particle internalization, enzyme activation, cellular transport or translocation or any other measure of endogenous receptor function.

Of critical importance is that the bead system may be used to test drugs and other cellular interventions and therapies by inhibiting the signaling proteins by pharmacological methods or using knock-out cells or cells where RNA expression of the putative signaling proteins has been silenced by interference RNA. The effect of the therapies or interventions may be measured directly by the failure of the cellular pathway to function in terms of the production of metabolic products or translocation of known signaling proteins to the bead or by some other measure of the function of the activated receptor including protein phosphorylation, particle internalization, enzyme activation, cellular transport or translocation or any other measure of receptor function.

The method may be used to:

(I) Isolate and identify intact membrane associated protein complexes of activated receptors;

(II) Compute the proteins specifically associated with receptor ligand coated beads or other coated beads;

(III) Confirm the presence and interaction of the MS/MS identified proteins at the site of the ligand beads by independent biochemical, immunological or microscopic methods;

(IV) Determine the role of the receptor complex protein and associated proteins in receptor function; and

(V) Determine the penetration, efficacy, kinetics and side effect of drugs or therapeutic molecules on receptor function.

The ligand may be bound to the bead by hydrophobic or electrostatic interactions or by covalent bond via carboxyl or amino or epoxy or by cyanogen bromide or other activation. Proteins or antibodies or peptides or small molecules or drugs or lipids, carbohydrates, nucleic acids, proteins, singly or in combination with other ligands may be covalently or non-covalently bonded to the bead. The bead or surface may be plastic, polypropylene or other polymers, PVDF, nitrocellulose, glass, normal phase or other. Proteins or antibodies can be adhered to PVDF that has been activated in methanol or organic solvents. Normal phase surfaces could be acid washed, or ethanol washed or other and coated with poly-lysine or some other polymer or other functional groups attached to silanol bonds or other bonds on the bead or surface. The proteins might also be dried onto the surface and held by electrostatic forces. Antibodies could be bound to protein G or protein A or covalently attached to the surface and might serve as the ligand directly or hold the ligand. The surface may be used as is, or modified with linking reagents such as poly-lysine, or protein cross-linkers, or esters or ether linkages or via silanol bonds or the like other bonds. The bead may have functional moieties for coupling ligands or have been derivatized via silanol bonds or other bonds. The beads may be derivatized or alkylated or methylated or alkanated or alkenated or acylated, or derivated with any variety of chemical groups. The bead/surface could be reacted with glutaraldehyde, or paraformaldehyde, N-hydroxy succinimide or sulpho-NHS, or other thiol cross-linker such as soluble N-ethylmaleimide-cross linking reagent. The crosslinking reagent will link once to surface and once to some other protein and thus covalently attach them to bead. The bead could be reacted with cleavable or non-cleavable bi-functional reagent. The beads may be coated with polymers or a natural or synthetic source. The ligand may be attached to the beads by chemical moieties including amine, carboxylic, biotin-streptavidin, silanol, polylysine, NHS or other silicon based chemistries with or without spacer arms. The bead may be particles such as a live or dead cell with or without fixation that carry a specific ligand or have been modified by the attachment of proteins or other biomolecular complexes. Thus the protein, or peptide, or antibody or small molecule or an antibody or a protein complex or a nucleic acid polymer or carbohydrate or lipid or a small molecule or drug or a complex of any of the above could then be attached to the bead or surface.

Phagocytic cellular functions are mediated through multi-ligand receptor families of proteins, glycoproteins or glyco-lipo protein complexes that are expressed on the surface of cells and cooperate to regulate cellular functions. Cellular functions include the response to ligands that promote growth and differentiation of cells and that activate or regulate cellular metabolism. Receptors also may regulate the movement of ligands and other materials into the cell. The intent of the present invention includes but is not limited to the uses of ligand coated beads that address the cellular functions associated with the phagocytic functions of cells including cellular movement and engulfment of particles.

Macrophages are a suitable model cell for phagocytic functions since macrophages can infiltrate tissues and move towards target cells and particles, and can engulf and ingest those particles while secreting destructive factors and producing oxygen radicals. Many serious diseases including the development of atherosclerotic plaques, cancer and Alzheimer dementia involve the misdirected movement of cells that can engulf other cells or tissues, secrete factors to alter their environment and infiltrate tissues.

Fatty streaks or other sources of lipid particles in the arteries may be engulfed by phagocytic receptors to yield giant foam cells that contribute to the root causes of atherosclerosis. A variety of diseases including atherosclerosis depend on the functions of cell surface receptors to trigger their onset or progression and recovery. The innate immune system is the first line of defense against microbial infections and other infectious diseases. Innate immune signals from scavenger, bacterial and antibody receptors seem to share overlapping signaling mechanisms. However, little is known with certainty about the identity and exact isoforms of the shared signal recognition and response machinery that regulate phagocyte behavior in response to infection and during inflammation that destabilizes the microenvironment around atherosclerotic plaques and other lesions that may be the direct trigger of serious disease. The activation and rupture of these plaques lead to heart attacks and strokes.

There is an urgent need to understand the precise mechanisms controlling signaling pathways leading to phagocytosis and endocytosis that may result in the engulfment of OX-LDL or IgG bearing particulates. The recognition of molecular patterns and the coordination of the host response is apparently arranged by protein complexes surrounding the surface receptors on the plasma membrane.

Macrophages possess many scavenger receptors with broad specificity and low affinity that examine extracellular complexes alongside high affinity receptors that specialize in determining the cargo's fate and triggering the best host response. The arrangement of host receptors mirrors the ligands on the particle. The host cells likely make a complex mosaic of receptors and their effectors at the site of recognition that provides for a wide variety of responses depending in part on the particle's ligands and size. Identification of the signaling molecules associated with the CD36 and the Fc receptor which mediate phagocytic actions will markedly improve our understanding of inflammatory signaling networks. Full knowledge of these will have a profound impact on our ability to prevent or treat heart attack, infectious diseases, stroke, cancers, arthritis, neurodegeneration and many other diseases and is of great importance.

A very important part of the knowledge required was to determine if the membrane and proteins required to engulf particles are derived from the new ER pathway or the previous classical endocytic pathway.

Cellular functions are mediated through multi-ligand receptor families of proteins, glycoproteins or glyco-lipo protein complexes that are expressed on the surface of cells and cooperate to regulate cellular functions. The response of phagocytes to engulf particles and produce reactive oxygen species is triggered and regulated by multi-ligand receptors on the cell surface including the Ig superfamily members such as the Fc gamma receptors and the scavenger receptors. Scavenger receptors include SR-A, CD36, CLA-1, CD68, LOX-1 and other Ig-domain-containing receptors such as cysteine rich macrophage scavenger receptors MARCS. The ligands that stimulate the activation of phagocytes via these families of multiligand receptors include hydrophobic surfaces such as polystyrene particles, OX-LDL, IgG, C-reactive protein, other modified lipids and apoptotic cells and potentially many other molecules or complexes that might be recognized as pathogen associated molecular patterns, i.e. “non-self” by the innate immune receptors. There is binding or functional data showing that CD36 and Fc receptors bind or cooperate with integrins and likely other surface receptors.

Leukocytes are white blood cells including macrophages and neutrophils that carry innate immune receptor such as scavenger receptors (SR), LPR receptors, bacterial receptors and Fc receptors that cooperate to engulf microscopic particles such as lipid aggregates. Atherosclerosis seems to require or adopt the innate, inflammatory signaling mechanisms of phagocytes to trigger onset or progression.

While excessive alcohol may prevent the function of anti-bacterial systems, these same mechanisms inhibited by alcohol may be responsible for the formation of atherosclerotic plaques. However, there is a paucity of information regarding the molecular mechanisms that regulate phagocyte behavior during inflammatory invasion that destabilizes the micro-environment around atherosclerotic plaques that may be the direct trigger of serious disease.

The two routes to foam cell formation are phagocytic engulfment of micro particles and fluid phase macropinocytosis of free lipids. Particle accumulation occurs via innate receptors producing giant foam cells. However, it has been recently shown that free fluorescent cholesterol may enter macrophages via macropinocytosis, perhaps without receptor mediation in highly activated cells. There is an urgent need to understand the precise mechanisms controlling the PLD pathway leading to phagocytosis, and perhaps the related macropinocytosis, that result in the accumulation of LDL and OX-LDL or their aggregates.

Lipid signal pathways similar to macropinocytosis and phagocytosis are responsible for the conversion of macrophages into foam cells that form atherosclerotic plaques and block arteries. The three main lipid signal pathways that regulate particle engulfment are the PI3K & PLC pathways leading to PiP3 & DAG and the PLD pathway leading to PA and DAG. While there is general agreement that the PI3K pathway is a therapeutic target and regulates both phagocytosis and macropinocytosis, less is known about PLD and there are previous publications that do not show that PAP-1 directly regulates the oxidative burst, but rather the opposite, that PAP-1 is a negative regulator of the oxidative burst. Our data show the opposite of the previously published data, we show a pharmacologically characterized PAP-1 activity is the direct regulator of the oxidative burst.

Leukocytes, including macrophages and foam cells, have innate immune receptors including bacterial receptors, scavenger receptors (SR) and Ig superfamily receptors that seem to work together and share some common signaling response mechanisms. Upon binding to inflammatory ligands, a number of intracellular biochemical events are initiated that culminate with innate phagocyte responses that may include particle engulfment and the activation of the oxidative burst. The oxidative burst is the production of superoxide anions by an enzyme complex termed the phagocytic oxidase or NADPH oxidase associated with the membrane bound organelle called the phagosome that forms around particles and cells as they are ingested by phagocytes such as neutrophils and macrophages. The convenient physical connection of the ligand receptors, PLD, and the oxidative machinery in the forming phagosome presents an attractive target for the use of sensitive LC/LC-MS/MS and live cell confocal enzyme assays to detect and measure the presence and function of proteins such as the receptor pathway proteins at the site of the activating particle. Since receptors may show lateral mobility, the receptor and its associated proteins may accumulate at the site of the ligand coated bead.

Many receptors may cooperate to engulf particles and other cellular functions. The use of ligand coated beads permits the binding and integration of many receptor pathways in a single experimental event.

There is a paucity of evidence on the exact isoforms of many receptor associated proteins and molecular mechanisms that engulf lipids into macrophages to create foam cells, still many lines of genetic, clinical and animal model data confirm that macrophages and CD36 are essentially required for the development of atherosclerosis.

Hypercholesterolemic mice become resistant to atherosclerosis if bred to macrophage deficient strains. Atherosclerotic plaques form when low-density lipoproteins containing cholesterol bind to the surface of the arteries perhaps via peptideoglycans where they become oxidized or otherwise altered to present as themselves as Pathogen Associated Molecular Patterns, i.e. “non-self”, to the innate immune system via CD36. Thus macrophages can be activated in response to the signals of injury including the presence of oxidized phospholipids and other lipids that may act as molecular mimics of bacterial surfaces. Monocytes contact and infiltrate the wall of the blood vessel beneath the forming plaque and mature into macrophages with the accompanying expression of CD36. The macrophages express MPO and NADPH oxidase enzymes as well as lipoxygenase and rapidly convert available LDL to OX-LDL. The transition to foam cells is accompanied by the expression of the CD36, CLA-1 and CD68. The macrophage cells accumulate and sequester oxidized cholesterol and lipids via innate immune receptors including CD36 producing giant foam cells. Unsaturated fatty acids, for example the omega-6 polyunsaturated fatty acids, are transported into macrophages by CD36 and result in the expression of cyclooxygenase and the release of the highly inflammatory 2 series of prostaglandins.

The action of cyclooxygenase is required for the initiation of the atherosclerotic plaque formation in mice. Ligation of innate immune receptors stimulates the expression of cyclooxygenase and release of arachidonic acid. Upon activation, macrophages engulf their targets and metabolize the production of oxygen free radicals that lead to further production of OX-LDL, oxyphospholipids and oxysterols, and ingest surrounding lipid aggregates via innate receptors. In addition, antibodies against oxidized lipid and against phospholipids may permit the similar accumulation of lipids in immuno-complexes via the Fc receptor.

There is evidence that uptake of OX-LDL into atherosclerotic plaques via innate immune receptors such as CD36 receptors is at least as efficient as uptake via immunoconjugates and that binding of oxidized phospholipids to the opsonin C reactive protein would permit their direct uptake via the Fc gamma receptor. In this proposal, the engulfment of lipid particles and immuno-complexes via the CD36 or Fc gamma are modeled using magnetic or polystyrene particles coated with OX-LDL or IgG will likely reflect much of the range of cooperative signaling systems in atherosclerotic plaques. In addition to atherosclerosis, CD36 and Fc receptors have been implicated in the activation of macrophages and microglia associated production of reactive oxygen species, phagocytosis and cellular attack in Alzheimer's dementia.

Inflammatory damage to cartilage and joints is known to require the function of the Fc receptors in mice. Hence recent evidence indicates that the cellular signals associated with the innate immune systems of the phagocytes are required for a broad range of serious inflammatory diseases.

Endocytosis is a clathrin dependant process that occurs with soluble and aggregated ligands or nanoparticles: Phagocytosis is an Actin dependant process that occurs in larger micro particles. Recent experiments in the areas of drug delivery and material science have shown that the engulfment of nano particles on the order of 0.1 micron results from clathrin dependant. Moreover the amount, rate and kinetics of the particle engulfment is remarkably dependant on particle size and the ligands bound to the particle surface. The instant inventors have recently shown that it is possible to mine the hundreds of proteins associated with particles bearing different receptor ligands including IgG and OX-LDL and that the kinetics of these two particle uptakes are remarkably different.

We have used ligand coated microparticles to specifically capture and sequence the proteins of the plasma membrane by proteomics. From these many recent studies there is every good reason to believe that coating 0.1 micron particles with nothing, IgG and OX-LDL will trigger the authentic mechanisms of endocytosis of these soluble ligands and that coating 2 micron particles with these ligands will stimulate the phagocytosis of these ligand coated particles.

The proteome of the phagosome of un-coated polystyrene particles has been partially elucidated by the relatively insensitive and laborious 2D gel electrophoresis method. However, negative control experiments, such as the identification of the proteins from crude extracts or growth media that interact non-specifically with the particles were not presented.

On the basis of this data, it has been hypothesized that phagocytotic engulfment occurs via the endoplasmic reticulum (ER); that proteins are not degraded by the cathepsins and proteases known to exist abundantly in the phagosome; but rather proteins are exported from the phagosome degraded by the proteasome and the resulting peptides returned to the phagosome for MHC presentation. In contrast, a wealth of cell biological data indicates that the vesicular pathway provides the membrane and proteins required to engulf particles. Recently, the development of tandem liquid chromatography coupled to tandem mass spectrometry (LC/LC-MS/MS) has made it possible to identify the proteins associated with a patch of activated membrane.

Many lines of evidence confirm that macrophages and innate immune responses are essentially required for the development of atherosclerosis. Hypercholesterolemic mice become resistant to atherosclerosis if bred to macrophage deficient strains. Atherosclerotic plaques form when low-density lipoproteins containing cholesterol bind to the surface of the arteries perhaps via peptideoglycans or otherwise altered to present themselves as Molecular Patterns to the innate immune system via CD36/SR . Thus macrophages can be activated in response to the signals of injury including the presence of oxidized phospholipids and other lipids that may act as molecular mimics of bacterial surfaces. Monocytes contact and infiltrate the wall of the blood vessel beneath the forming plaque and mature into macrophages with the accompanying expression of CD36/SR. The macrophages express MPO and NADPH oxidase enzymes as well as lipoxygenase and rapidly convert available LDL to oxLDL. The transition to foam cells is accompanied by the expression of the CD36, CLA-1 and CD68. The macrophage cells accumulate and sequester oxidized cholesterol containing micro particles via innate immune receptors including CD36/SR producing giant foam cells. Unsaturated fatty acids, for example the omega-6 polyunsaturated fatty acids, are transported into macrophages by CD36/SR and result in the expression of cyclooxygenases and the release of the highly inflammatory prostaglandins. The action of cyclooxygenase (COX) is required for the initiation of the atherosclerotic plaque formation in mice. Ligation of innate immune receptors stimulates the expression of cyclooxygenase and release of arachidonic acid. Upon activation, macrophages engulf their targets and synthesize super oxide radicals that lead to further production of OX-LDL, oxyphospholipids and oxysterols, and ingest surrounding lipid aggregates via innate receptors. In addition, antibodies against oxidized lipid and against phospholipids may permit the similar accumulation of lipids in immuno-complexes via the Fc receptor. There is evidence that uptake of OX-LDL or apoptotic cells into atherosclerotic plaques via innate immune receptors such CD36/SR receptors is as efficient as uptake via immuno-conjugates although the binding of oxidized phospholipids to the opsonin C reactive protein would permit their direct uptake via the Fc gamma receptor. In this proposal, the engulfment of aggregated LDL or IgG coated micro particles, free DI-LDL will likely reflect the much of the range of cooperative signaling systems in atherosclerotic plaques.

### DETAILED DISCUSSION OF FIGURES AND EXPERIMENTAL PROCEDURES

We have used RAW 264.7 cells and human neutrophils as model systems that engulf ligand coated polystyrene beads via CD36, scavenger receptors or via the Fc receptor. We performed a complete examination of the polystyrene beads incubated in culture medium, crude lysates poured over un-opsonized and IgG or OX-LDL opsonized beads as controls. We used LC/LC-MS/MS to identify large number of proteins specifically associated with the phagosomes of human neutrophils and RAW 267.4 murine macrophages after isolation by ultra-centrifugation.

CD36 is an integral plasma membrane glycoprotein that shows significant homology with the Drosophila Croquemort protein that is required for the phagocytosis of apoptotic cells with altered surface lipids and homology to the neuronal sensory protein Snmp-1. How CD36 might signal is presently not clear. CD36 has very little cytoplasmic tail that might be used for classical protein-protein interaction experiments such as affinity chromatography or two hybrid screens. CD36 contains similarity to a molecule that contains a RUN domain that is similar to domains required for interaction with Ras superfamily members of the Rab and Rap class. It is possible that the domains CD36 requires to transmit signals are located on its binding partners. CD36 is often classified as a class A scavenger but shows significant homology to members of the class B scavenger receptors, cholesterol ester and fatty acid transporters, and lysosomal integral membrane proteins. CD36 cooperatively binds with Integrins and Thrompsondin-1 that in turn binds with integrin activating protein. Thus recent evidence indicates that CD36/SR function as a signaling protein required for the engulfment of hydrophobic molecules by phagocytes. While it is clear that phagocytosis is dependant on PI3K and Ras super family pathways the exact class and isoforms responsible are not known and much less is known about CD36 mediated endocytosis. There is also evidence that the Fc may be directly involved in the accumulation of aggregated or altered lipids bound by antibodies or C reactive protein. Stimulation of human macrophages with OX LDL has lead to the increased expression of the Fc receptors and CD36 receptors: The nature of innate immunological synapse at the site of OX-LDL binding is also not clear but evidence with authentic human macrophages in vitro indicate that the Fc and CD36 receptors are involved in the engulfment of OX-LDL particles by authentic human macrophages.

There is no data available to clearly determine the specific class and isoforms of PI3K, for example, and many other proteins required for the uptake of particles by the innate immune receptors. A role of the class I p110 beta subunit in cell migration and phagocytosis has been shown by antibody micro injection alone. However specific requirement for the Class I p110 alpha and beta sub-units in endocytosis of OX-LDL and phagocytosis of IgG coated micro particles by silencing RNA remain to be shown. In addition class II PI3K has recently been shown to function in cell migration but their role in particle engulfment is unknown. Statins are a family of drugs that inhibit HMG-CoA reductase resulting in the inhibition of the isoprenoid pathway and a reduction in serum cholesterol, or other cellular isoprenoids, associated with a 30% to 50% decrease in the rate of heart attack, but there is evidence that statins may interrupt the prenylation of small G proteins of the Ras superfamily. The reduction in the production of isoprenoids may have many pleiotropic effects on the cells including a reduction in the isoprenoid groups that anchor signaling proteins similar to Rac/CDC42. The role of the Ras superfamily in the engulfment of different types of microscopic particles is not yet well defined.

Lovastatin is one of the most popular anti-atherosclerosis drugs of the statin family. Propranolol, but not all other beta blockers, has been shown to prevent heart attack by about 35% similar to the effect of statins. Moreover, there is evidence that statins may function via effects other than merely lowering cholesterol. The reduction in the production of isoprenoids may have many pleiotropic effects on the cells including a reduction in the isoprenoid groups that anchor signaling proteins similar to Rac/CDC42 that may regulate PLD. Recently the effect of statins on lowering cellular activation and oxidative stress has been linked to its effect on the PLD pathway. It has been assumed that the effect of propranolol was due to a by-product of beta adrenergic blockade. However, some other even more effective beta blockers do not show the effect of preventing heart attack indicating that it may be a property other than its beta blocking activity that is the source of the heart attack preventing effect of propranolol. We have shown that an enzyme matching the pharmacological profile of PAP-1 seems to play a permissive role in the regulation of the oxidative burst and phagocytosis. Many studies agree that balance of PLD and PAP-1 activity directly or inversely regulates cellular response depending on agonist receptors, timing, dose and cell types. The structurally unrelated Mg²⁺ PAP-1 inhibitor HELSS was used as an additional PLD/PAP-1 pathway inhibitor. HELSS has a side effect of inhibiting iPLA2 that can be controlled for using the iPLA2 inhibitors MAFP and AACOCF3. Our preliminary data point to a common effect of ethanol, statins and propranolol in preventing particle accumulation of macrophages. Given that ethanol can inhibit heart attack in manner similar to the results reported for statins and that lovastatin and the PLD/PAP-1 pathway inhibitors ethanol and propranolol all prevent the engulfment of particles by macrophages we propose to examine the role of the PLD/PAP-1 pathway in the engulfment of LDL or IgG coated polystyrene micro particles and the macropinocytosis of free DI-LDL.

The PLD pathway has been strongly linked to phagocytic accumulation of particles and the NAPDH oxidative burst but in vitro experiments at 500 micromolar propranolol showed little effect. Recently it has been shown that ethanol, 30 micromolar HELSS and 1 mM propranolol all had the same effects on live cells and thus demonstrated a role for PLD and PAP-1 in cyclo-oxygenase expression and macrophage functions.

We have shown for the first time that under the conditions used by Dennis ethanol, propranolol and HELSS all behave in the same manner potentially unifying a disparate literature. We show for the first time that the activation of PLD leads to the production of phosphatidic acid that is in turn converted to diacylglycerol by the action of PAP-1 at the site of micro particle engulfment. Thus in our foam cell model, different PLD pathway inhibitors ethanol, propranolol and HELSS all prevent the engulfment of micro particles similar to statins. Numerous studies have shown that alcoholic beverages protect against atherosclerotic diseases such as heart attack.

We have devised a model system of cultured macrophages that utilizes uptake of micro particles through innate immune receptors to create giant foam cells. Foam cells are the central component of atherosclerotic plaques that clog arteries, and upon activation and apoptosis by the presence of oxidized lipids, lead to plaque rupture and heart attack or stroke. We have shown that alcohol has a very similar effect to the other drugs know to prevent heart attack, the statins and propranolol. Alcohol is the only known inhibitory drug of phospholipase D (PLD). PLD is required for particle engulfment and the generation of free radicals by leukocytes. Moreover, propranolol, that has recently been shown to prevent heart attack, has also been shown to inhibit a downstream effector of the PLD pathway, Mg⁺⁺ dependant phosphatidic acid phosphohydrolase (PAP-1). Furthermore, statin drugs have recently been implicated in effecting small G proteins of the type that are associated as effectors of the PLD pathway.

The preliminary data indicate that we have discovered the shared mechanism by which alcohol and statins prevent heart attacks. We have shown that ethanol and statins both prevent the accumulation of hydrophobic micro particles by macrophages and similarly inhibitors of the PLD pathway, ethanol, propranolol and HELSS all prevent particle engulfment and the generation of oxidative free radicals by leukocytes. Together, we feel that the effect of alcohol on the PLD pathway may provide the solid mechanistic basis of the role of alcohol on the prevention of heart attacks.

As an enabling demonstration beads coated with IgG, the ligand for the Fc receptor, were incubated with live macrophage cells that normally consume IgG opsonized particles as part of their physiological function. The beads were incubated with the live cells on ice for 30 minutes to permit receptor ligation and clustering at the site of the activating bead. Subsequently the cells were warmed to 37 degrees for 5 minutes to permit the formation of the activated receptor complex. The activation of the receptor complex was monitored by confocal microscopy to determine that activation started within several minutes of warming and was complete by 5 minutes of warming. Beads were sampled at each time by collection with a rare earth magnet or by centrifugation with or without a sucrose gradient in a buffer containing 140 mM KCl and 10 mM glucose and 1 mM MgCl. The beads were subsequently subjected to liquid chromatography followed by digestion of proteins with proteases and mass spectrometry. The liquid chromatography consisted of eluting the beads with 150, 200, 250, 300, 350, 400, 450, 500, 600 and 1000 mM NaCl. The elution fraction and the exhausted beads were digested with trypsin in 5% acetonitrile for the eluants and in 60% organic solvent for the insoluble beads. The tryptic digests were subsequently analyzed by mass spectrometry.

### DETAILED DESCRIPTION OF THE FIGURES

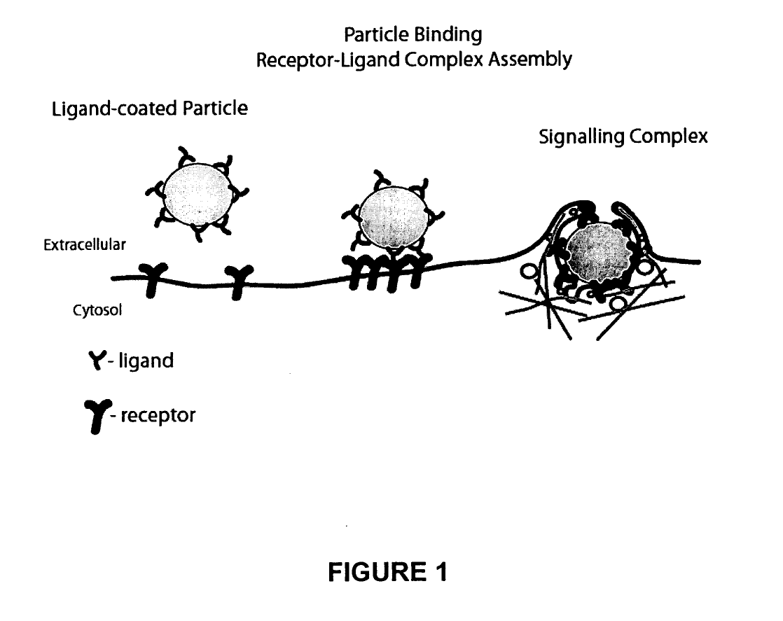

FIG. 1 describes a process wherein ligands presented on microscopic beads to live cells stimulate formation of receptor complexes at or near the surface of the cell and enables initiation, formation and elucidation of signaling complex over time.

Ligands presented on microscopic beads to live cells stimulate formation of receptor complexes at or near the surface of the cell (FIG. 1).

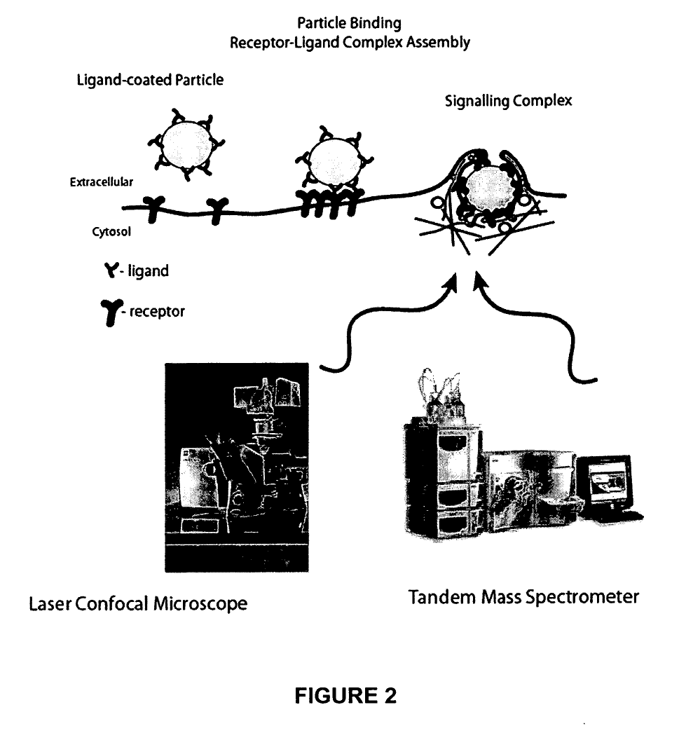

FIG. 2 illustrates the process of FIG. 1, wherein identification of the signaling complex is accomplished by the combination of confocal microscopy and mass spectroscopy. (see legend). Two of the most powerful technologies applied to biological discoveries are laser confocal microscopy and proteomic identification of proteins by tandem mass spectrometry (FIG. 2). Confocal microscopy permits in situ observation of proteins performing their cellular functions including interacting with other proteins to form cellular signaling complexes using cellular protocols and techniques. Proteomic identification permits direct elucidation of the identity of proteins within cellular signaling complexes using biochemical protocols and techniques

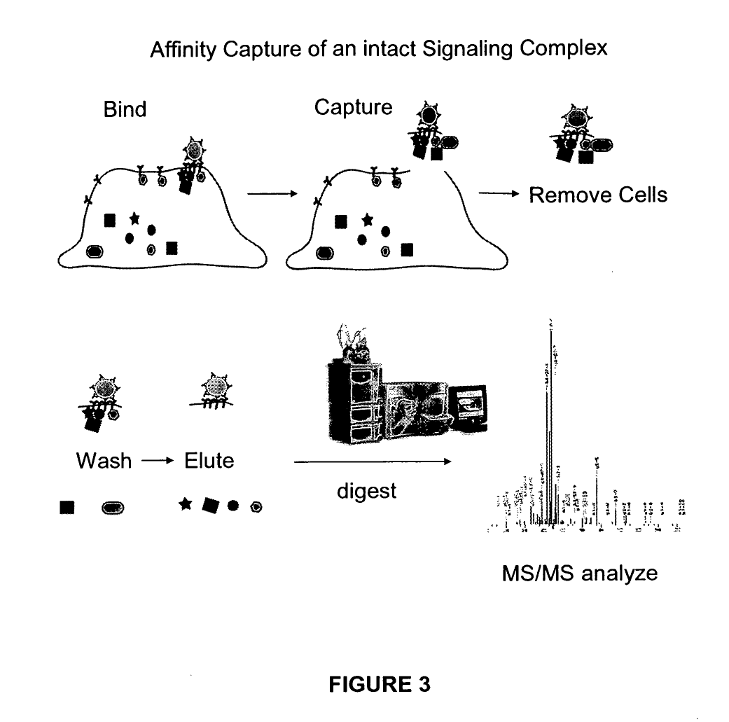

FIG. 3 illustrates a strategy for capturing a patch of membrane containing an activated and assembled receptor complex. The beads bound via their ligands to cellular receptors can be collected from the live cells or after disruption and identified by mass spectrometry.

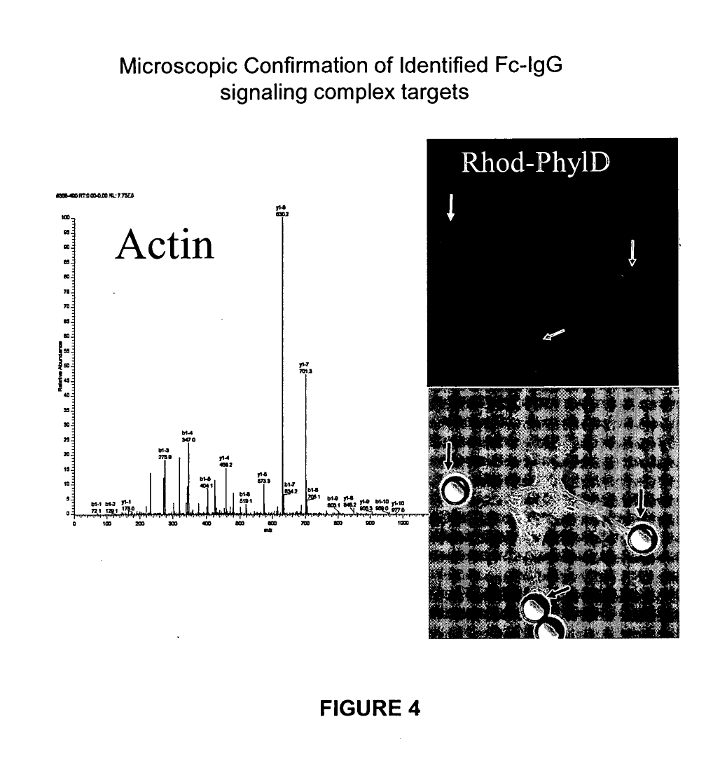

FIG. 4 illustrates the instant process for verification and identification of signaling complex protein, using mass spectroscopy and confocal microscopy, wherein mass spectroscopy is initially used to verify the presence of a particular protein, subsequent to which, in the second validation stage, the same beads can be used to verify the participation of the discovered proteins by confocal microscopy in a quantitative and qualitative manner thus unifying these two powerful technologies, as in the example of Actin (FIG. 4). The bead thus serves as the link between cell biology and mass spectrometry with a self-validation step built into the process. In the second validation stage, the same beads can be used to verify the participation of the discovered proteins by confocal microscopy in a quantitative and qualitative manner thus unifying these two powerful technologies as in the example of Actin (FIG. 4).

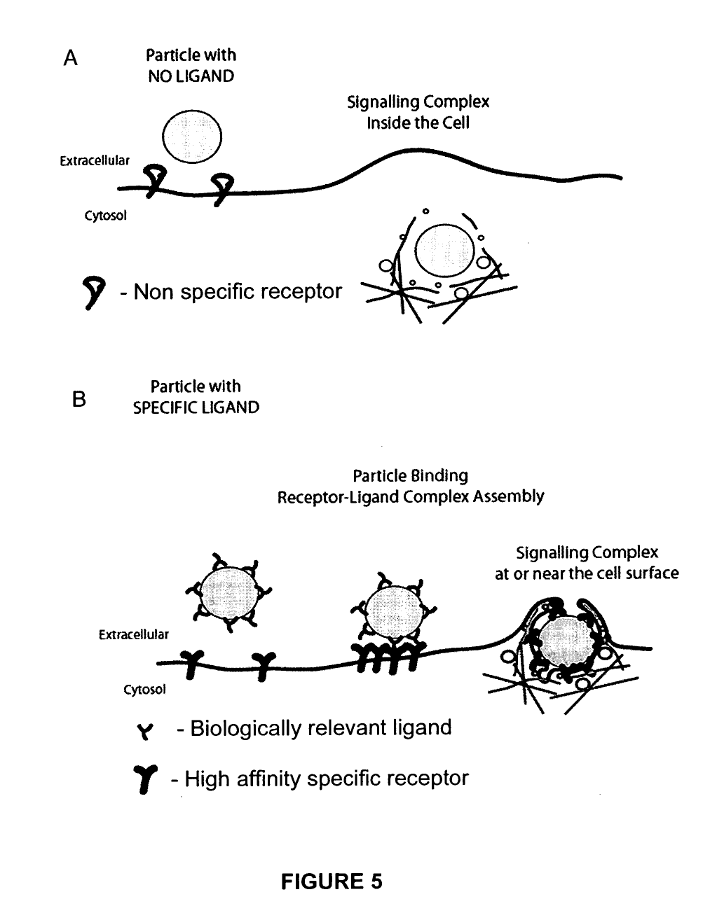

FIG. 5, panels A and B, respectively, distinguish internalization of phagosomes (naked beads) versus surface receptor binding of ligand bound beads specifically bound to relevant receptors. These two technologies, mass spectrometry and confocal microscopy, have already been combined using beads without ligands (FIG. 5, A).

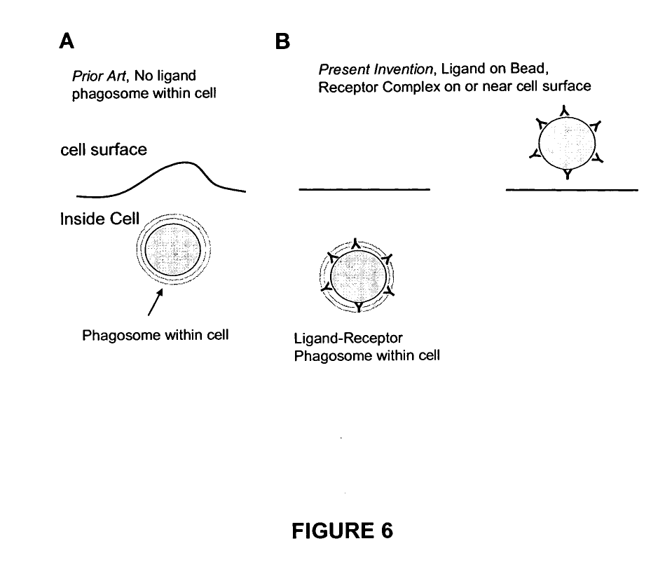

FIG. 6 illustrates the difference between the prior art process wherein engulfment of naked beads, to form phagosomes, occurs within the cell, as opposed to the instant invention, wherein receptor complexes bound to ligand coated beads can be elucidated on or near the cell surface, or within the cells. Mass spectrometry and confocal microscopy, have already been combined, using beads without ligands, to examine the internalized phagosome, a membrane bound organelle within phagocytic cells, 30 minutes after engulfment. The present invention teaches the use of ligand coated beads bound at or near the cell surface (FIGS. 6 A and B).

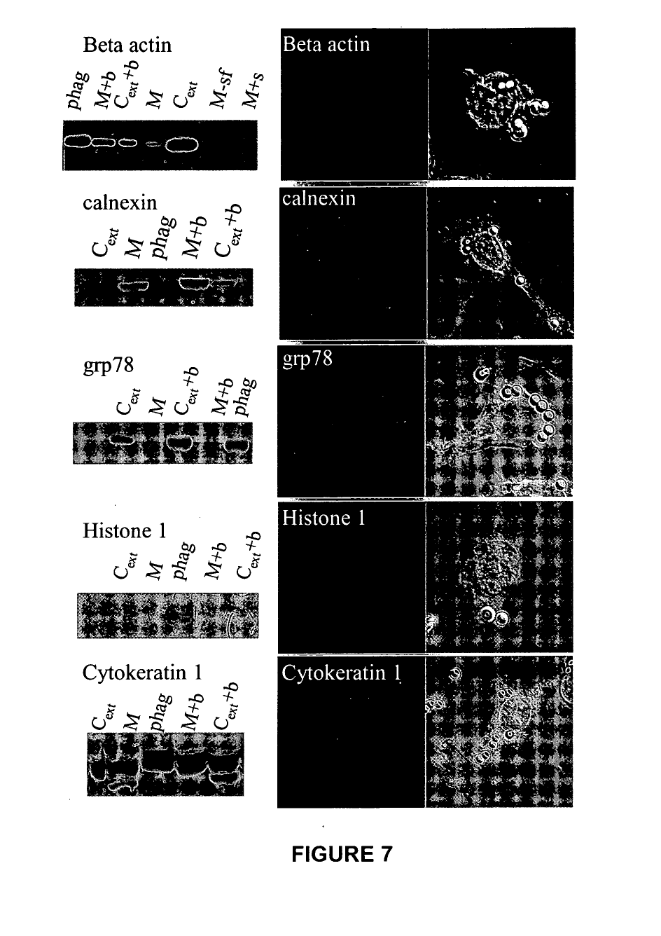

FIG. 7 shows use of confocal microscopy, biochemical and immunological methods to differentiate between non-specific high abundance proteins and those proteins which form strong signaling complexes which bind to the ligand coated bead. Use of the prior art showed that calnexin was concentrated in the phagosome and that the endoplasmic reticulum itself and not other drug targets directly effects phagocytosis of un-modified particles. We observed no accumulation of calnexin at the phagosome but did observe calnexin in the growth media and found that calnexin strongly binds to and accumulates on bead without ligands. No calnexin accumulation was observed on beads previously blocked with a ligand such as IgG. Similarly no accumulation of GRP 78 was observed at the site of the ligand coated beads compared to that of Actin (FIG. 7).

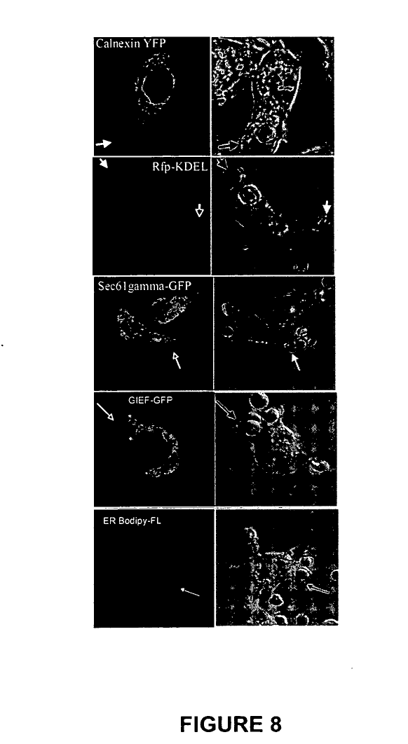

FIG. 8 illustrates failure of the endoplasmic reticulum (ER) proteins to play a role in the receptor complex formulation. We expressed GFP fusion constructs of proteins associated with the endoplasmic reticulum (ER) such as the luminal ER marker KDEL, calnexin, Sec 61 gamma, the ribosomal sub-units GIEF or general ER staining with ER bodipy to examine the association of the ER with the forming phagosome. At no time during the formation of the phagosome did we observe the ER markers or general ER stain co-localize anywhere near the initial membranes that formed around the engulfed particles. (FIG. 8) After engulfment was complete, rather than showing an increased concentration of ER proteins at the site of the engulfed particles as would be expected if they provided the membranes, a shadow or gap in the ER around the particle location was observed. Hence we found no evidence that the endoplasmic reticulum pathway found by the prior art played a role in modified, ligand coated beads, at or near the cell surface.

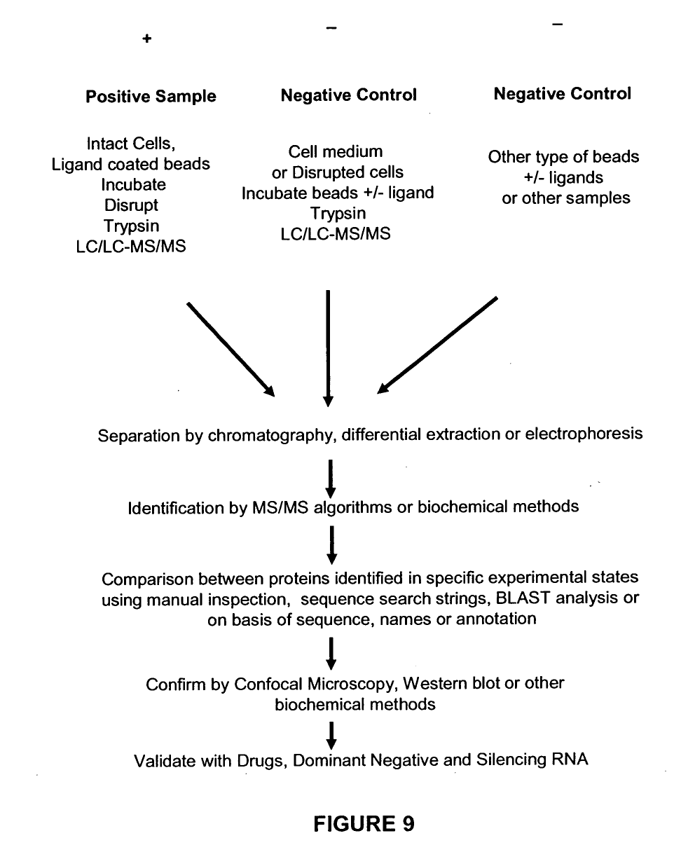

FIG. 9 is a work flow diagram using positive and negative controls to illustrate isolation, identification, confirmation and validation of receptor complex proteins that are specifically associated with ligand coated beads. In contrast to the prior art or uncoated or unmodified beads, instead of detecting apparent endoplasmic reticulum proteins the present invention detected the proteins associated with the signal pathway proteins of the Fc receptor (FIG. 9) and new novel drug targets not previously detected have been verified.

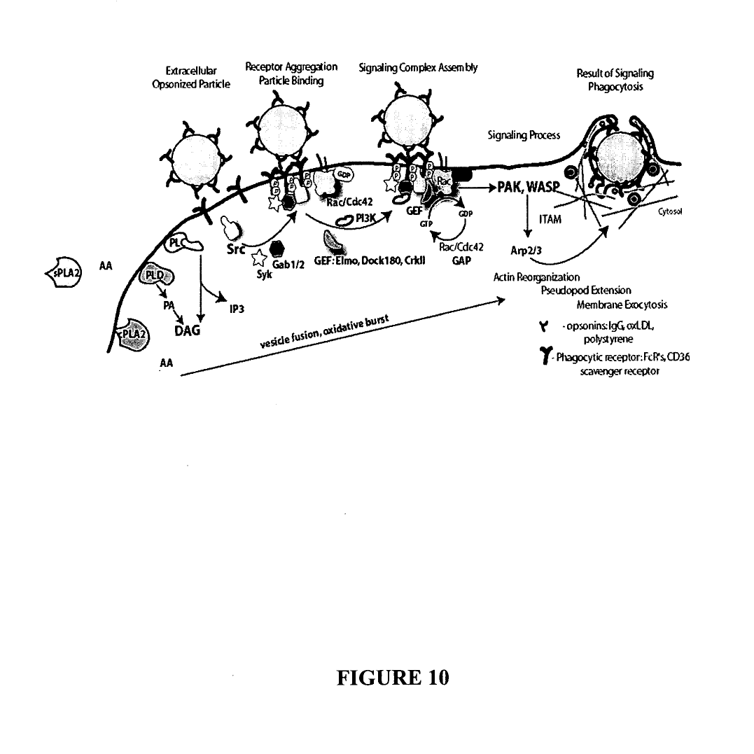

FIG. 10 is a molecular model of the signaling network that controls engulfment of particles presenting the Fc receptor ligand IgG. This model has been developed using cytogenetic and genetic mutation studies in mammals and other model systems.

The present invention seeks to effect the capture and identification of activated signaling complex on the cell surface and its associated protein complex drug target(s) by mass spectrometry and verifies that the identified proteins are functionally associated with the receptor using confocal microscopy or other biochemical assays by a simple and rapid method. The approach is to put the activating ligand of the signal receptor complex on a bead and allow the bead to interact with the cell of interest. The activation of the signaling complex may be measured in the cells by observing known signaling proteins translocating to the bead or by measurement of the metabolic products of the signaling pathway with a confocal microscope (or by some other measurement) at the ligand-coated bead. Once the time required for the beads to activate (or in-activate) the signaling complex upon introduction of the ligand-bead has been determined, the beads may be collected for discovery and assay of drug target proteins including, albeit not limited to, the types of receptor associated biopolymers such as those shown in (FIG. 10).

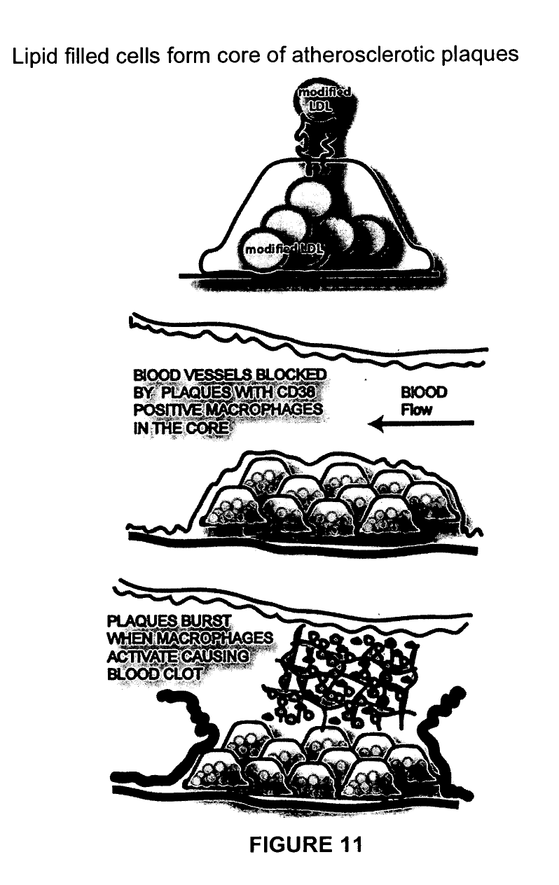

FIG. 11 illustrates the mechanism wherein receptor complex driven engulfment of modified particles generate lipid filled foam cells which form the core of atherosclerotic plaques. Fatty streaks or other sources of lipid particles in the arteries may be engulfed by phagocytic receptors to yield giant foam cells that contribute to the root causes of atherosclerosis. A variety of diseases including atherosclerosis depend on the functions of cell surface receptors to trigger their onset or progression and recovery.

The innate immune system is the first line of defense against microbial infections and other infectious diseases. Innate immune signals from scavenger, bacterial and antibody receptors seem to share overlapping signaling mechanisms. However, little is known with certainty about the identity and exact isoforms of the shared signal recognition and response machinery that regulate phagocyte behavior in response to infection and during inflammation that destabilizes the microenvironment around atherosclerotic plaques and other lesions that may be the direct trigger of serious disease.

The activation and rupture of these plaques lead to heart attacks and strokes. There is an urgent need to understand the precise mechanisms controlling signaling pathways leading to binding, receptor activation, inactivation that may result in the cellular responses, activations, inhibitions or suppression in response to modified particles or ligand coated particles including but limited to engulfment of modified particles of LDL, OX-LDL, modified LDL or IgG or other ligand bearing particulates (FIG. 11).

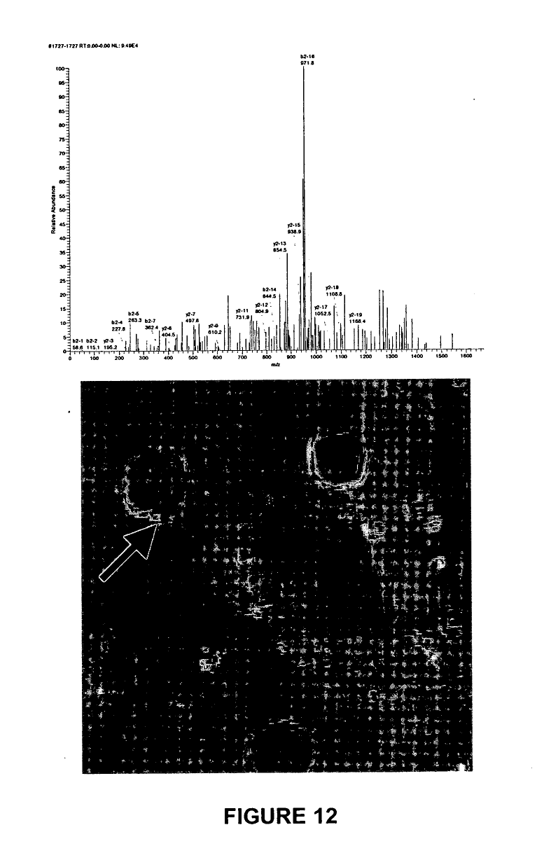

FIG. 12 Illustrates that the Fc Receptor (red) collects at site of ligand coated bead (arrow). Receptors may show lateral mobility, the receptor and its associated proteins may accumulate at the site of the ligand coated bead. The convenient physical connection of the ligand to the accumulated receptors and their receptor associated biopolymers including the membrane and cytoskeleton with receptor associated proteins presents an attractive target for the use of sensitive LC/LC-MS/MS and live cell confocal enzyme assays to detect and measure the presence and function of proteins such as the receptor pathway proteins at the site of the activating particle (FIG. 12).

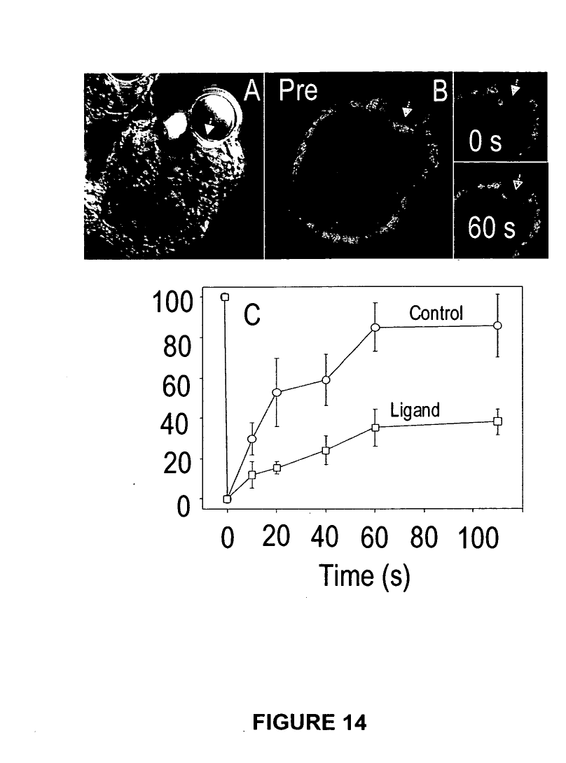

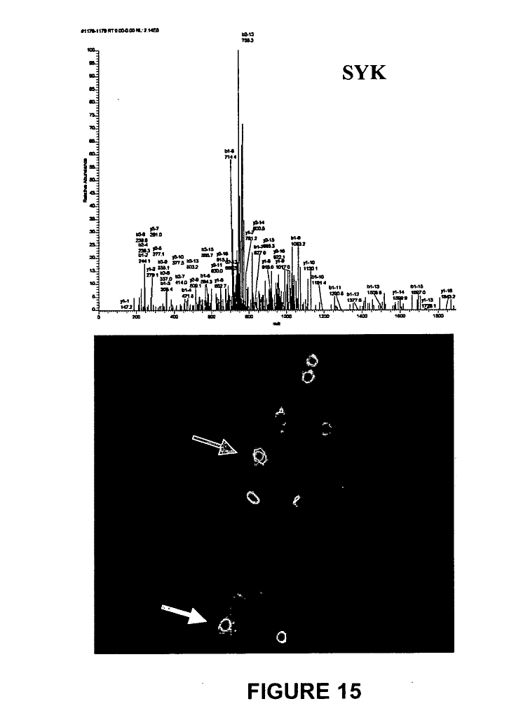

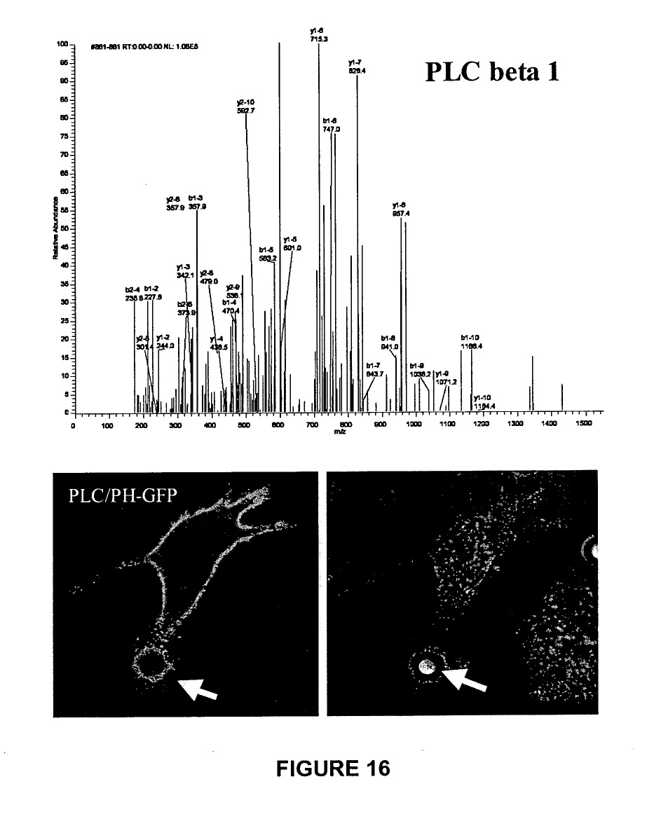

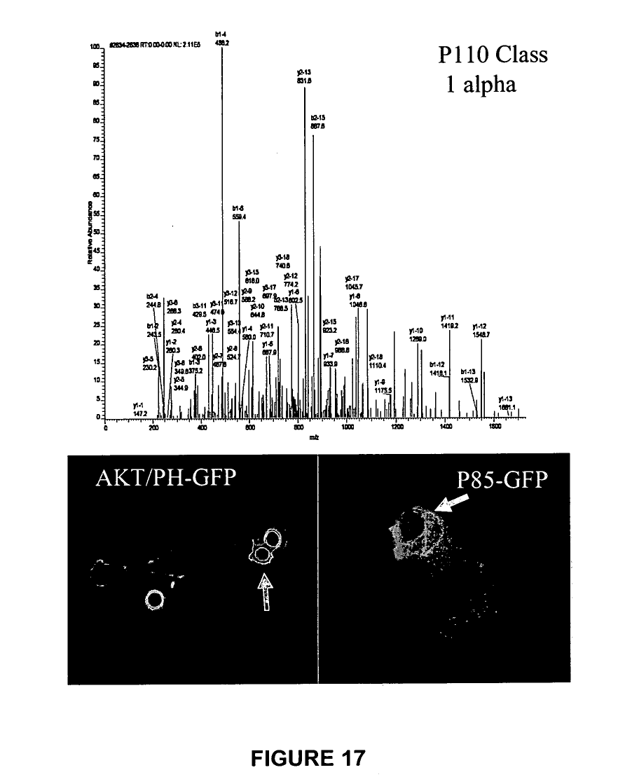

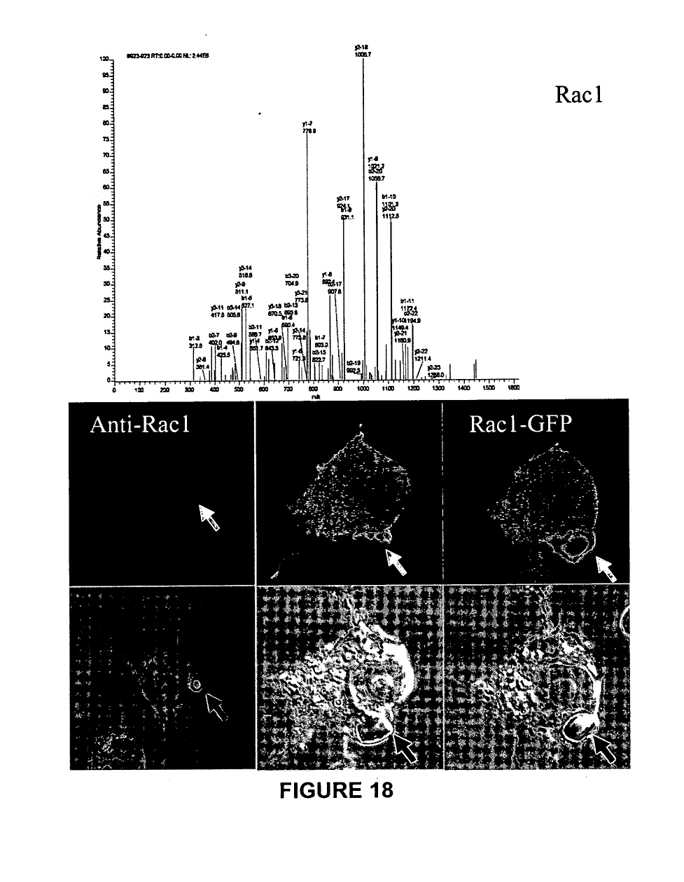

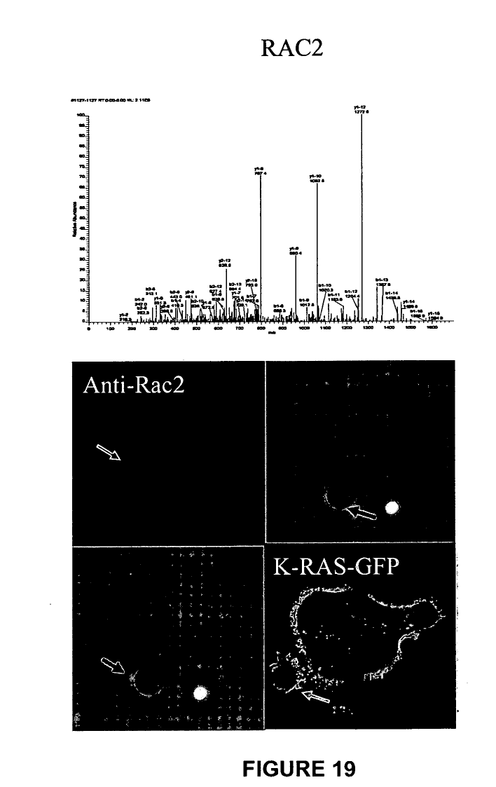

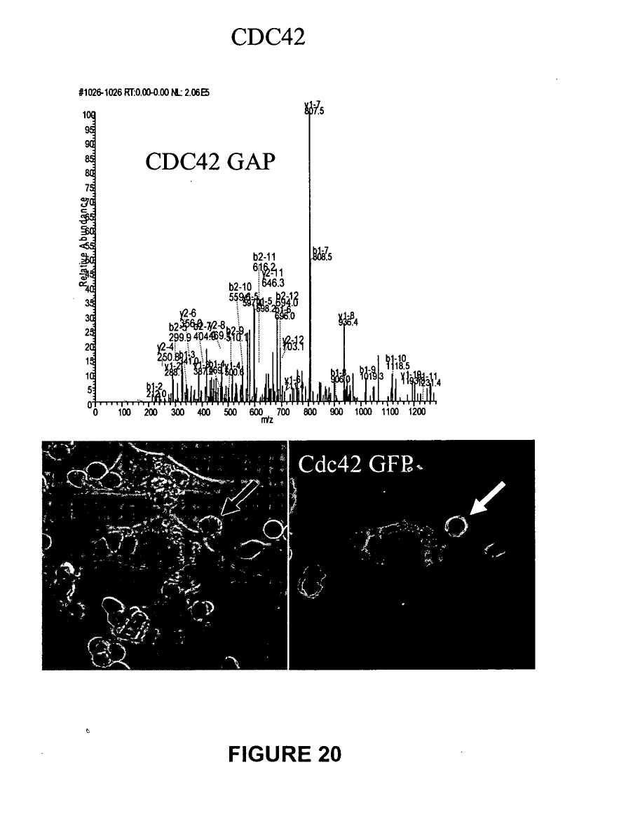

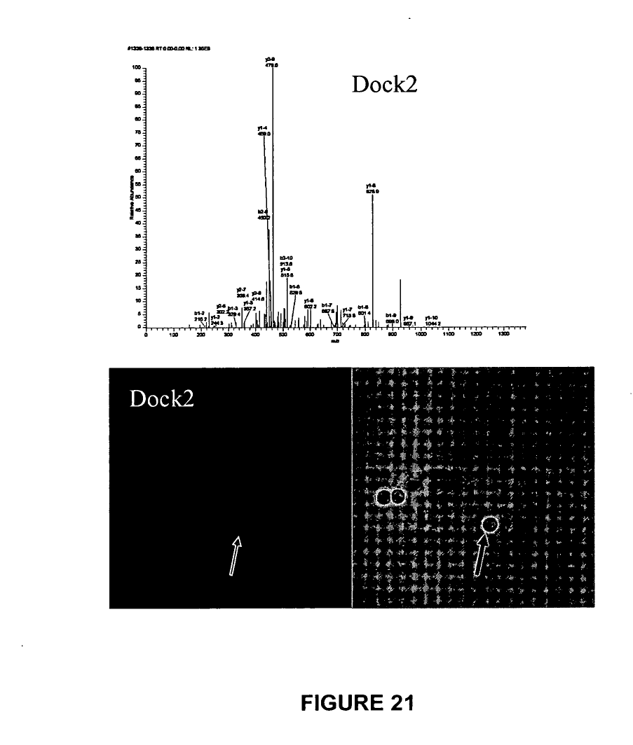

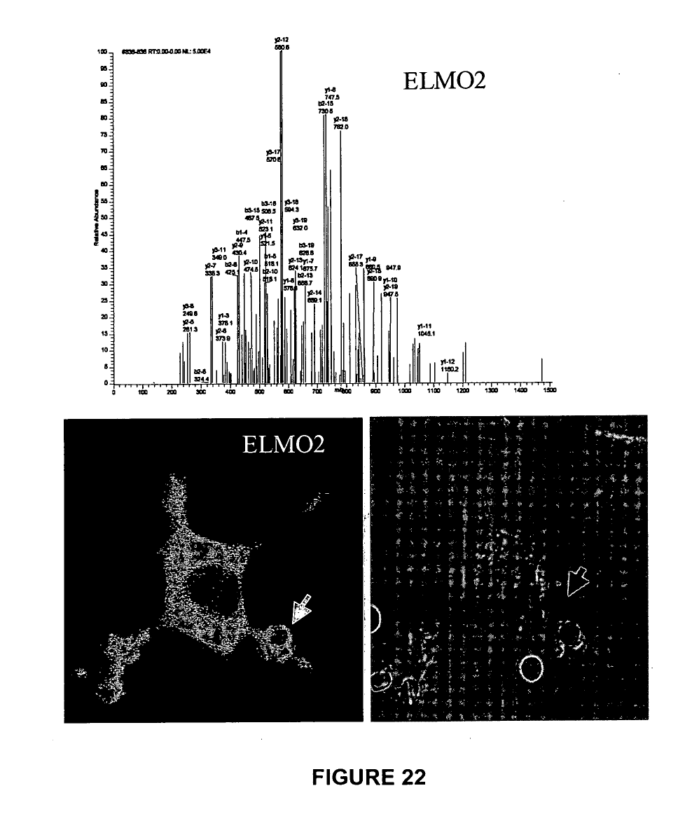

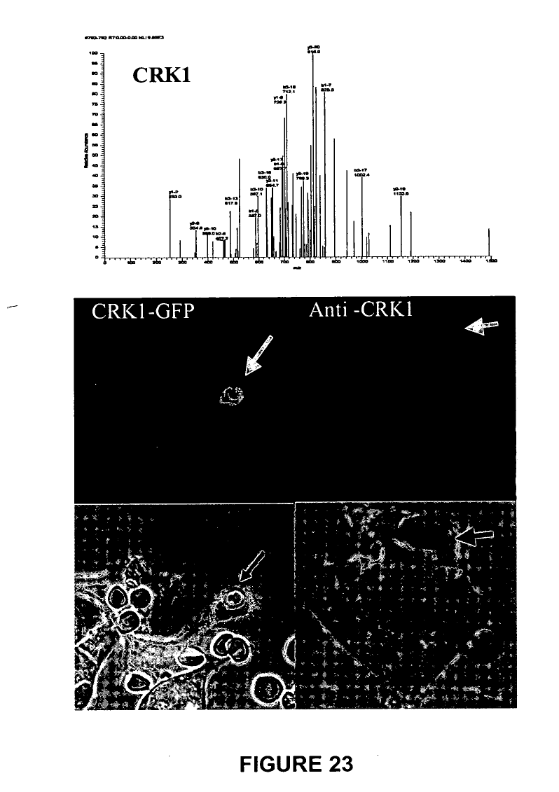

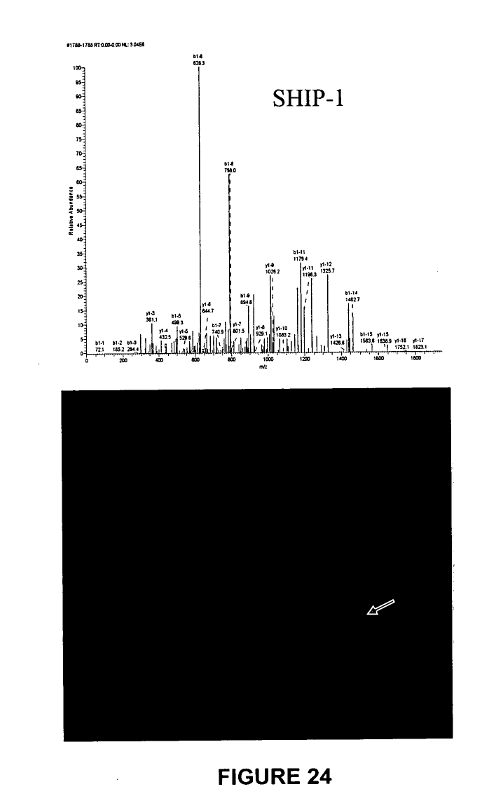

FIGS. 12-24 illustrate proof of principle that mass spectroscopically elucidated receptor complex proteins on or near the cell surface, specifically associated with ligand coated beads on or near the cell surface, can be independently verified to accumulate and bind at the site where the ligand coated bead activates the receptor by utilizing confocal microscopy using immunological reagents agents or fluorescent proteins. Moreover the proteins and biopolymers can be shown to participate in interactions at the site of the ligand coated bead at or near the cell surface using Florescence Recovery after Photo-Bleaching (FRAP) (FIG. 14).

The fraction of proteins specifically associated with the phagosomes, but not the negative controls, did contain specific isoforms of the signaling molecules associated with the vesicular model of phagocytosis including specific isoforms of Src, Syk, and their associated substrates, PLC, PLD, cPLA2, sPLA2, PI3K, RAS superfamily proteins such as CDC42/Rac, and their associated activating proteins (GAPs), exchanged factors (GEFs) including DOCK180, ELMO and regulators such as CRK (Table II, FIGS. 12-24)

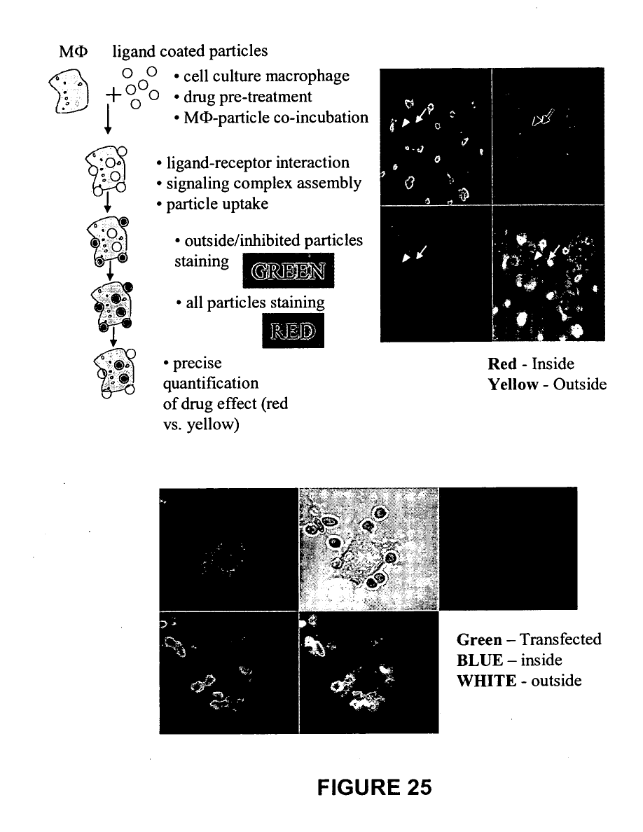

FIG. 25, panels A and B, illustrate respectfully a method for measurement of phagocytic receptors and a method for measuring transfection or delivery of nucleic acids and simultaneous measurement of particle engulfment using a single cell multi label confocal microscope experiment. A direct or surrogate measurement of receptor function or receptor pathway activity must be made in order establish the role of potential therapeutic target proteins in receptor function. IgG, complement, low density lipoprotein (LDL), oxidatively modified LDL (OX-LDL). Acetyl-LDL, apolipoproteins, lipoproteins, lipopolysaccharide (LPS), scavenger and other receptor functions can be measured by the accumulation of particles. The particles may themselves be comprised in part of fluorescent materials or can be directly stained with fluorescent or other colored materials or can be bound by proteins that directly or indirectly permit the attachment of fluorescent, chemiluminescent or other reporter molecules. The beads may be stained in live cells or cells fixed with formalin or paraformaldehyde or organic solvents such as alcohol and acids or by other means. The beads can be stained both before or after internalization with or without the permeabilization of the cells by detergents or organic solvents. The beads may be cells that can be lysed and imaged directly. The cells may be counter stained for the presence of specific proteins using antibodies or may express fluorescent protein constructs or contain fluorescent silencing RNA or DNA constructs or biopolymer modulating agents or drugs (FIG. 25).

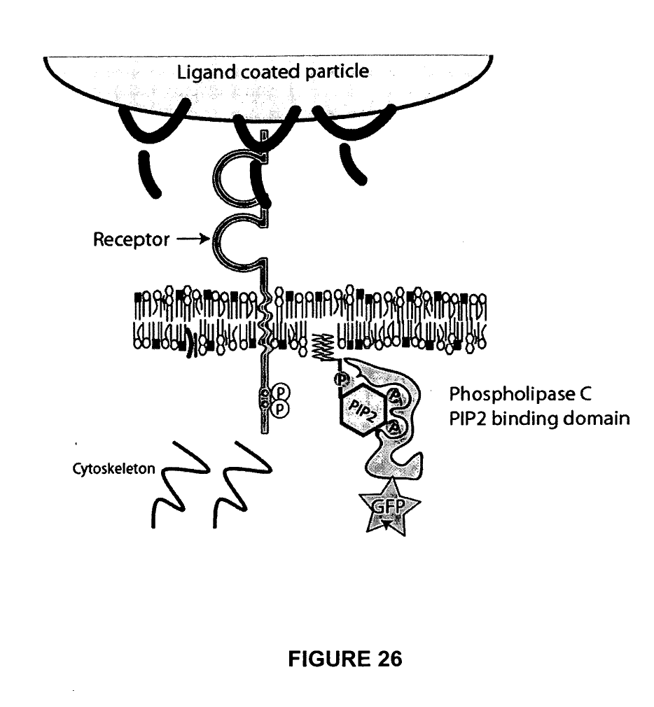

FIG. 26 illustrates the use of PiP2 binding domains to screen atherosclerosis drugs in macrophages. The metabolic activation of the receptor pathway at the binding site of modified beads such as ligand coated particles may be detected, measured and quantified using green fluorescence protein (GFP) fused with binding domains specific to different biopolymers, or modified biopolymers or metabolites in including phosphorylations at the site of modified particle or bead or binding. The binding domain may include albeit not limited to the phosphatidyl inositol bi phosphate (PIP2) binding domain such as that obtained from a protein, drug target biopolymer protein or biopolymer modulating agent or drug such as albeit not limited to the drug target protein PLC (phospholipase C) (FIG. 26).

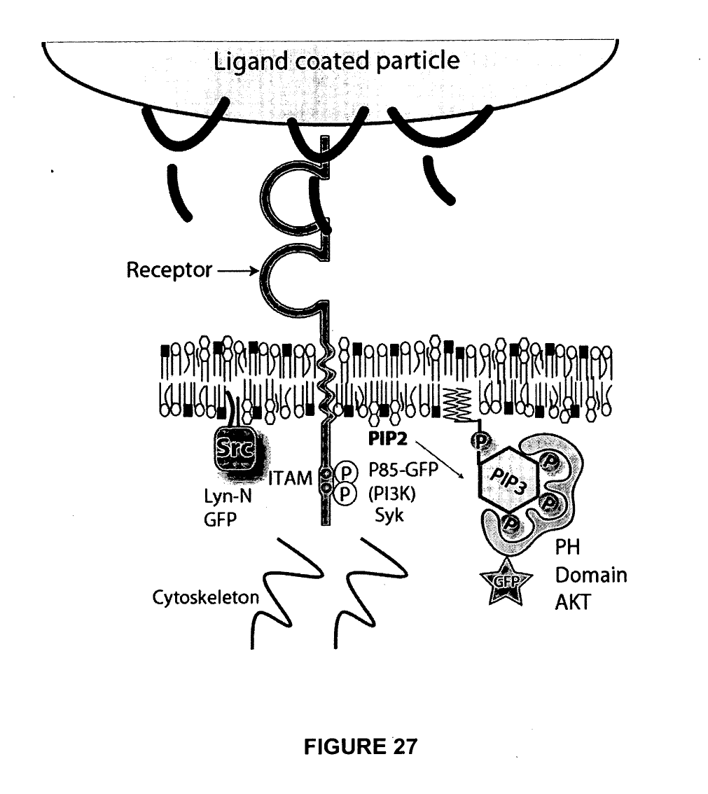

FIG. 27 illustrates the use of PiP3 binding PH domains to screen atherosclerosis drugs in macrophages. The metabolic activation of the receptor pathway at the binding site of modified beads such as ligand coated particles may be detected, measured and quantified using green fluorescence protein (GFP) fused with binding domains specific to different biopolymers, or modified biopolymers or metabolites in including phosphorylations at the site of modified particle or bead or binding. The binding domain may include, albeit not be limited to, the phosphatidyl inositol tri phosphate (PIP3) binding domain such as that obtained from a protein, drug target biopolymer protein or biopolymer modulating agent or drug such as albeit not limited to the drug target protein AKT (Protein Kinase B).

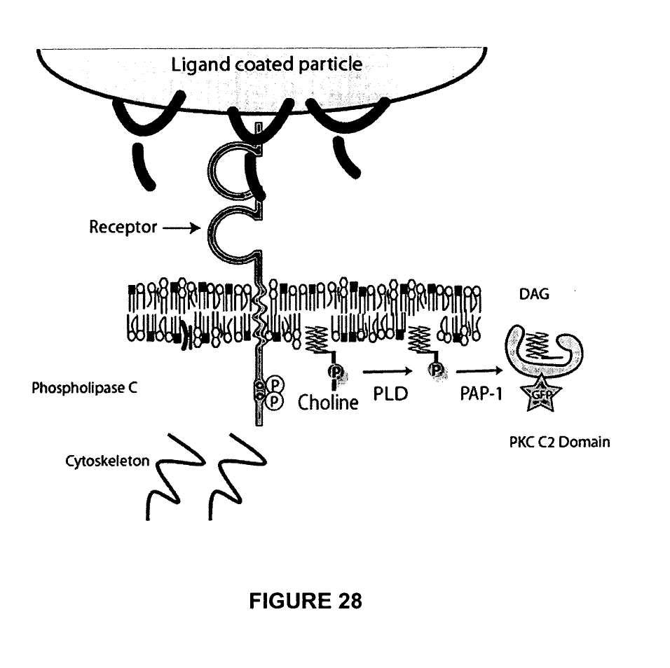

FIG. 28 illustrates use of DAG binding domains to screen for atherosclerosis drugs in macrophages. The metabolic activation of the receptor pathway at the binding site of modified beads such as ligand coated particles may be detected, measured and quantified using green fluorescence protein (GFP) fused with binding domains specific to different biopolymers, or modified biopolymers or metabolites in including phosphorylations at the site of modified particle or bead or binding. The binding domain may include albeit not limited to the diacyl glycerol (DAG) binding domain such as that obtained from a protein, drug target biopolymer protein or biopolymer modulating agent or drug such as albeit not limited to the drug target protein PKC (Protein kinase C).

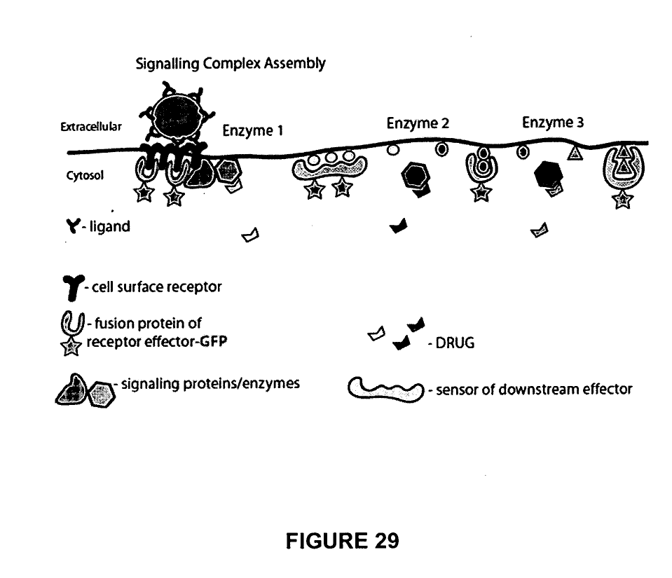

FIG. 29 illustrates monitoring of multiple metabolites or second messengers in series or parallel. The use of protein, biopolymer or metabolite binding domains fused to different molecules that have different light absorption or emissions properties could be used to monitor whole receptor pathways and at least one point simultaneously (FIG. 29).

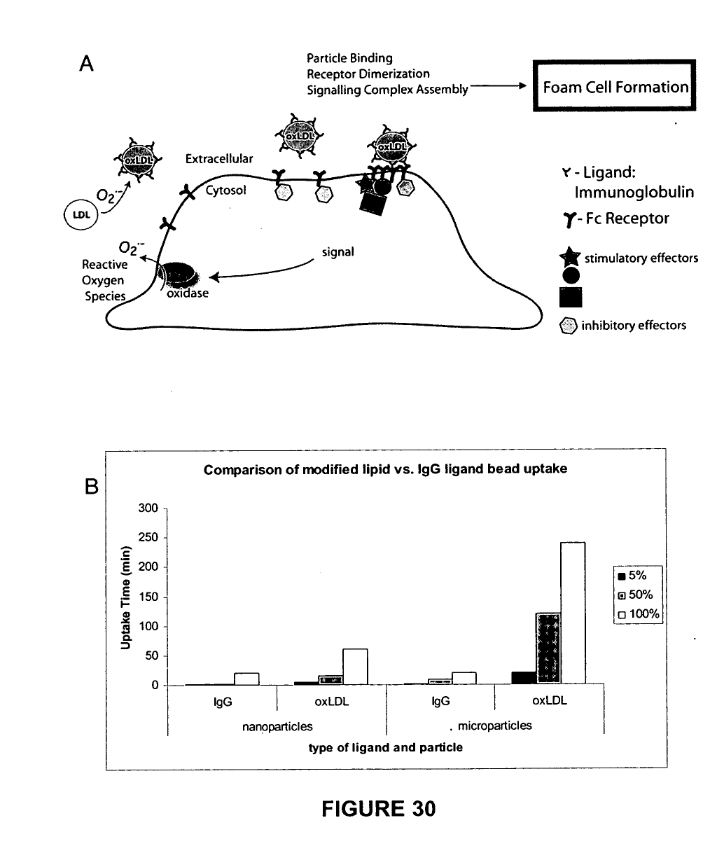

FIG. 30 illustrates modification of LDL to yield the ligand OX-LDL that binds receptors on the surface of macrophages. Many lines of evidence confirm that macrophages and innate immune responses are essentially required for the development of atherosclerosis. Hypercholesterolemic mice become resistant to atherosclerosis if bred to macrophage deficient strains. Atherosclerotic plaques form when low-density lipoproteins containing cholesterol bind to the surface of the arteries perhaps via peptideoglycans where they become oxidized or otherwise altered to present themselves as Molecular Patterns to the innate immune system via CD36/SR. Thus macrophages can be activated in response to the signals of injury including the presence of oxidized phospholipids and other lipids that may act as molecular mimics of bacterial surfaces. Monocytes contact and infiltrate the wall of the blood vessel beneath the forming plaque and mature into macrophages with the accompanying expression of CD36/SR. The macrophages express MPO and NADPH oxidase enzymes as well as lipoxygenase and rapidly convert available LDL to OX-LDL. The transition to foam cells is accompanied by the expression of the CD36, CLA-1 and CD68. The macrophage cells accumulate and sequester oxidized cholesterol containing micro particles via innate immune receptors including CD36/SR producing giant foam cells. Unsaturated fatty acids, for example the omega-6 polyunsaturated fatty acids, are transported into macrophages by CD36/SR and result in the expression of cyclooxygenases and the release of the highly inflammatory prostaglandins. The action of cyclooxygenase (COX) is required for the initiation of the atherosclerotic plaque formation in mice. Ligation of innate immune receptors stimulates the expression of cyclooxygenase and release of arachidonic acid. Upon activation, macrophages engulf their targets and synthesize super oxide radicals that lead to further production of OX-LDL, oxyphospholipids and oxysterols and ingest surrounding lipid aggregates via innate receptors. In addition, antibodies against oxidized lipid and against phospholipids may permit the similar accumulation of lipids in immuno-complexes via the Fc receptor. There is evidence that uptake of Ox-LDL or apoptotic cells into atherosclerotic plaques via innate immune receptors such CD36/SR receptors is as efficient as uptake via immuno-conjugates although the binding of oxidized phospholipids to the opsonin C reactive protein would permit their direct uptake via the Fc gamma receptor. In this proposal, the engulfment of aggregated LDL or IgG coated micro particles, free DI-LDL will likely reflect the much of the range of cooperative signaling systems in atherosclerotic plaques. We have used RAW 264.7 cells, J774 human neutrophils and Chinese hamster ovary cells as model Leukocyte systems that engulf ligand coated polystyrene beads via CD36, scavenger receptors or via the Fc receptor.

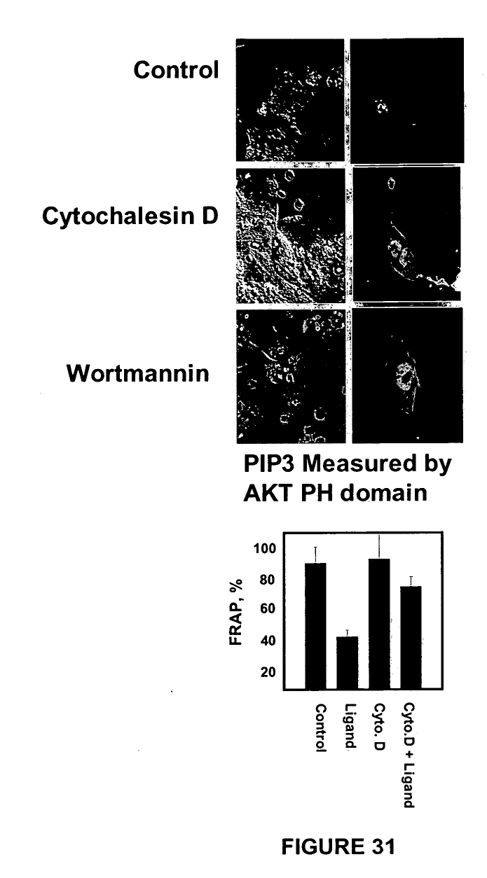

FIG. 31 describes utilization of genetically expressed fluorescent fusion protein domains as a measure of biopolymer function modulating material or drug on receptor signaling pathway function, at the binding site of ligand coated beads, on or near the surface of the cell. The accumulation of PIP3 at the site of IgG coated particles was inhibited using wortamannin, and cytochalasin D diluted into growth media (FIG. 31)

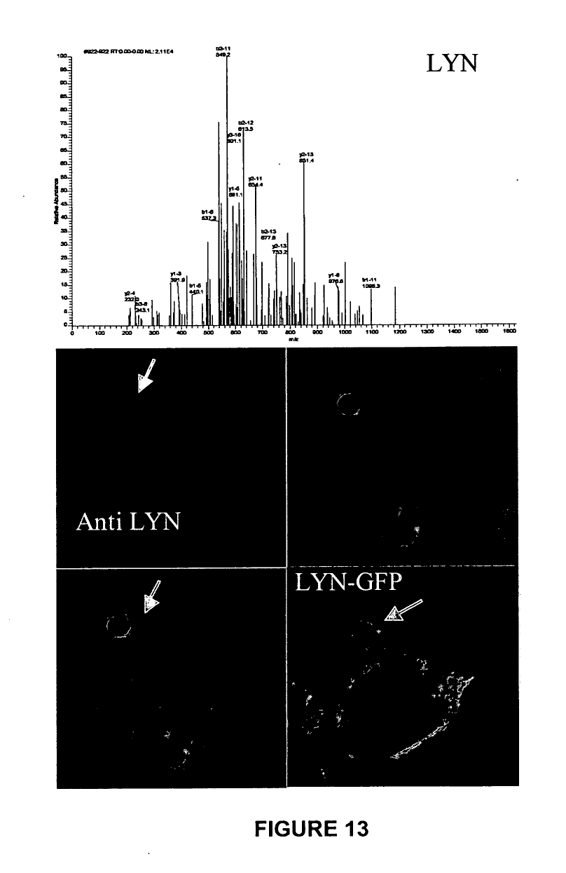

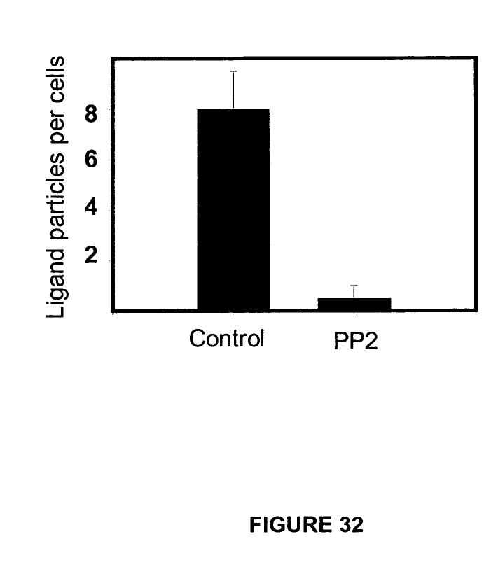

FIG. 32 shows use of a phagocytic receptor assay to screen effect of drug PP2 to inhibit the SRC proteins, instantly discovered by MS and confirmed by CF as described FIGS. 25 and 13. The engulfment of IgG coated particles was inhibited using the SRC kinase inhibiting drug PP2 diluted into growth media (FIG. 32).

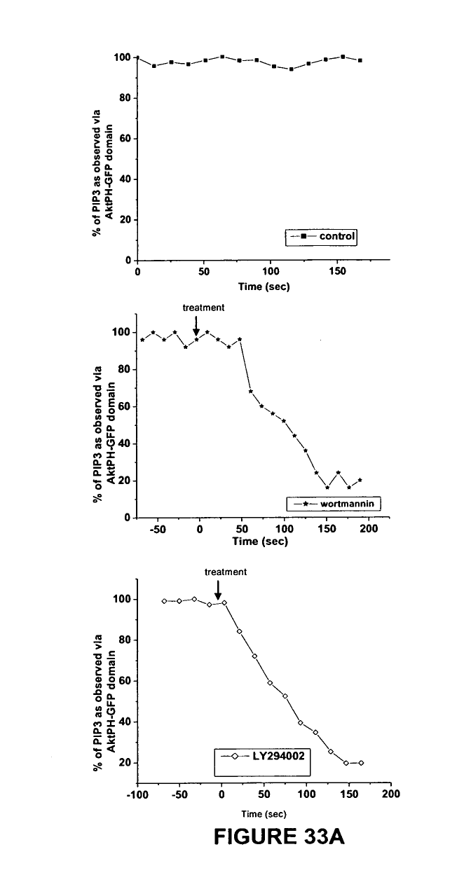

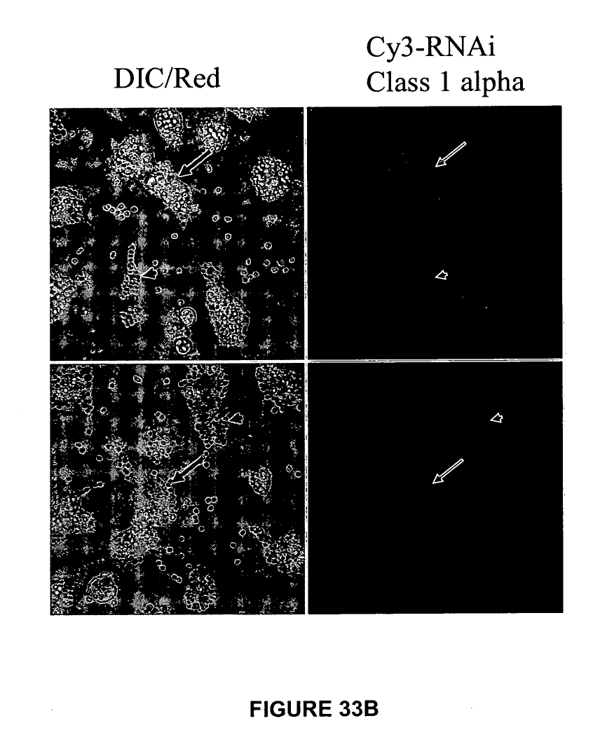

FIG. 33A, illustrates a quantitative interpretation of kinetics of PIP3 loss with wortmannin versus LY294002 using fluorescent protein domains; and FIG. 33B illustrates a visual interpretation of the loss of Fc receptor function following transfection of silencing RNA directed against PI3K class 1 alpha.

The PI3K pathway that converts PIP4, 5 bis-phosphate to PIP3, 4, 5, triphosphate, also called PIP3. PIP3 is measured by the Pleckstrin Homology (PH) domain of the protein kinase AKT, that has a high affinity for PIP3, fused to GFP (AKT/PH-GFP). The accumulation of phosphatidyl inositol triphosphate at the site of IgG coated particles was inhibited using wortamannin, and LY294002 dissolved into the cell experimental media.

The method permits the measurement of the penetrance, efficacy and dose response on kinetic of drug action at the site of an activated receptor (FIG. 33). The use of partially fixed red blood cells to bear a ligand coated bead also permits a quantification of the of effect of silencing RNA directed against PI3K Class I alpha against engulfment of modified or ligand coated particles. In this method the external particles are exploded in the presence of a hypotonic solution while the particles that have been engulfed are protected from lysis by the macrophage cell and can be counted directly (FIG. 33B).

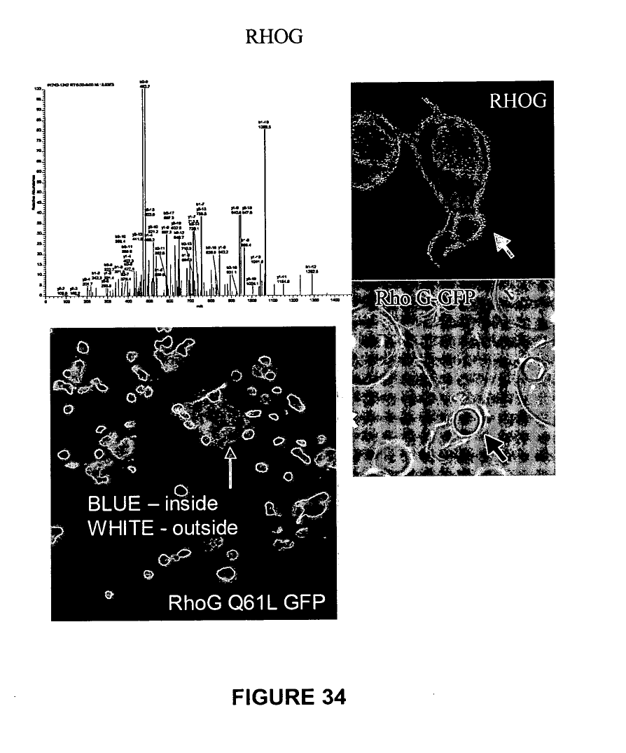

FIG. 34 at top left illustrates MS/MS spectra showing fragment ions for the 2+ peptide correlating to RhoG; Right Top: Expression of RhoG GFP in RAW macrophages, Note that RhoG localizes to the membrane that engulfs the particle, Bottom Left: Note that cell expressing dominant negative (green) RhoG in RAW macrophages has no (blue) engulfed particles. Bottom Right—DIC image showing location of the ligand coated bead, at or near the cell surface. The Ras superfamily has been shown to function in particle uptake and some isoforms of the Rac and CDC42 families have been shown to activate in phagocytic signaling, however little is known about the role of RhoG. RhoG has a cysteine residue in its N terminus and so, by homology, it could be expected to be held to the membrane by geranylation based on sequence similarity, and hence may be effected by statin drugs. Ras superfamily members or their regulatory proteins such as exchange factors or activating proteins may play a key role in particle engulfment.

We used cellular transfections of GFP dominant negative constructs of the small g proteins RhoG to demonstrate a functional requirement for the Ras superfamily in particle engulfment. We observed that dominant negative RHOG Q61L prevented the engulfment of IgG coated particles.

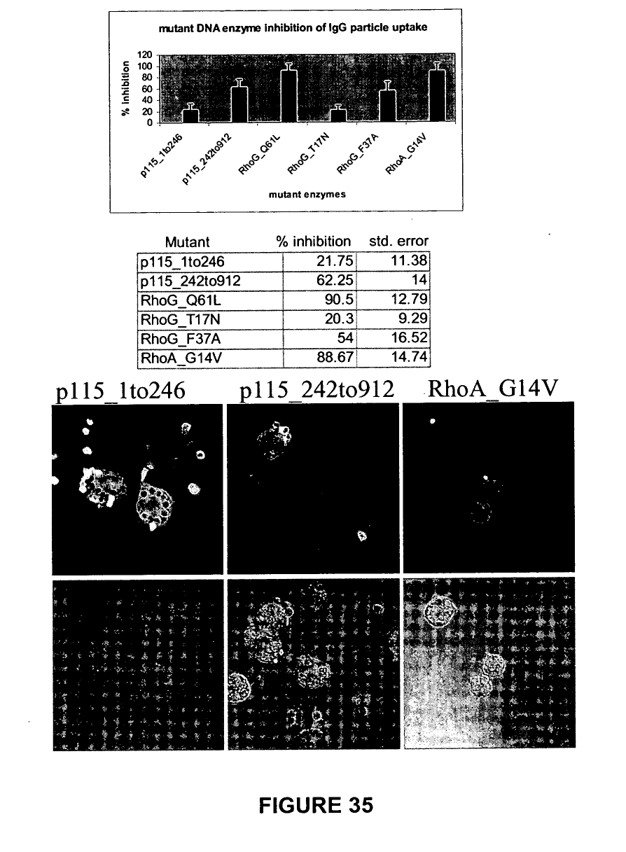

FIG. 35 shows use of phagocytic receptor assay to examine the effects of mutant nucleic acids. The RAS superfamily has been shown to function in particle uptake and some isoforms of the Rac and CDC42 families have been shown to activate in phagocytic signaling], however little is known about the role of RhoGEFs. RhoGEFs such as P115 exchange factors may play a key role in particle engulfment. We used cellular transfections of GFP dominant negative mutant nucleic acid polymer constructs of RhoA, RhoG, and P115 RhoGEF to demonstrate a functional requirement for the RhoGEFs in particle engulfment. Particles of Iron or polystyrene were coated in IgG or nothing. The particles were introduced to the growth media and incubated with RAW macrophages on ice and given time to settle and bind. The introduction to growth media will permit the binding of a broad range of undefined proteins to the surface of the beads and this could be avoided by the used of synthetic growth media if desired.

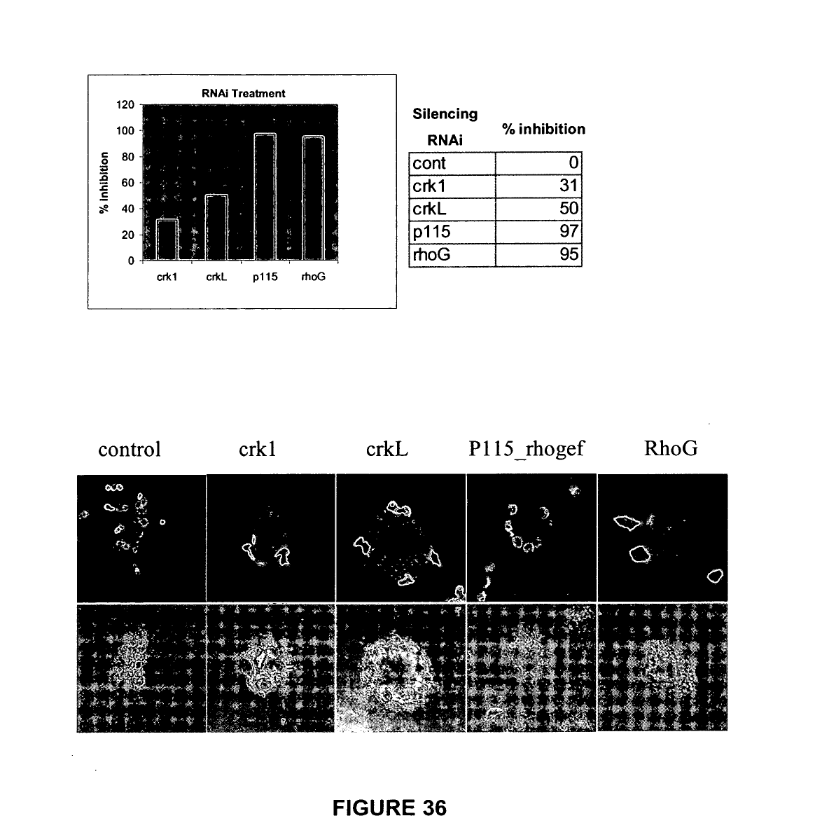

FIG. 36 use of phagocytic receptor assay to examine the effects of silencing RNA.

We used the biopolymer modification material silencing RNA against RhoA, RhoG, and P115 RhoGEF to demonstrate a functional requirement for the RhoGEFs in particle engulfment. Particles of Iron or polystyrene were coated in IgG or nothing. The particles were introduced to the growth media and incubated with RAW macrophages on ice and given time to settle and bind. The introduction to growth media will permit the binding of a broad range of undefined proteins to the surface of the beads and this could be avoided by the used of synthetic growth media if desired.

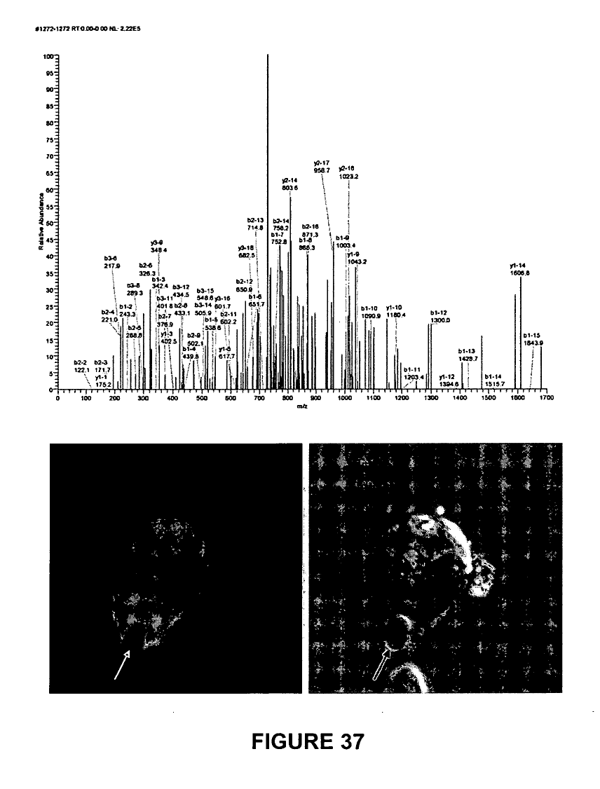

FIG. 37 illustrates proof of principle that mass spectroscopically elucidated RhoGEF on or near the cell surface specifically associated with ligand coated beads on or near the cell surface can be independently verified to accumulate and bind at the site where the ligand coated bead activates the receptor by utilizing confocal microscopy using fluorescent proteins. P115 RhoGEF was detected by mass spectrometry of ligands coated beads that had been bound to live cells and recovered prior to fractionation and digestion with enzymes and or chemical modifications prior to identification by mass spectrometry. Subsequently the accumulation of P115 RhoGEF at the site of ligand coated beads in live cells transfected with a fluorescent version of the discovered protein was used to confirm the presence of this protein in the activated receptor complex pathway.

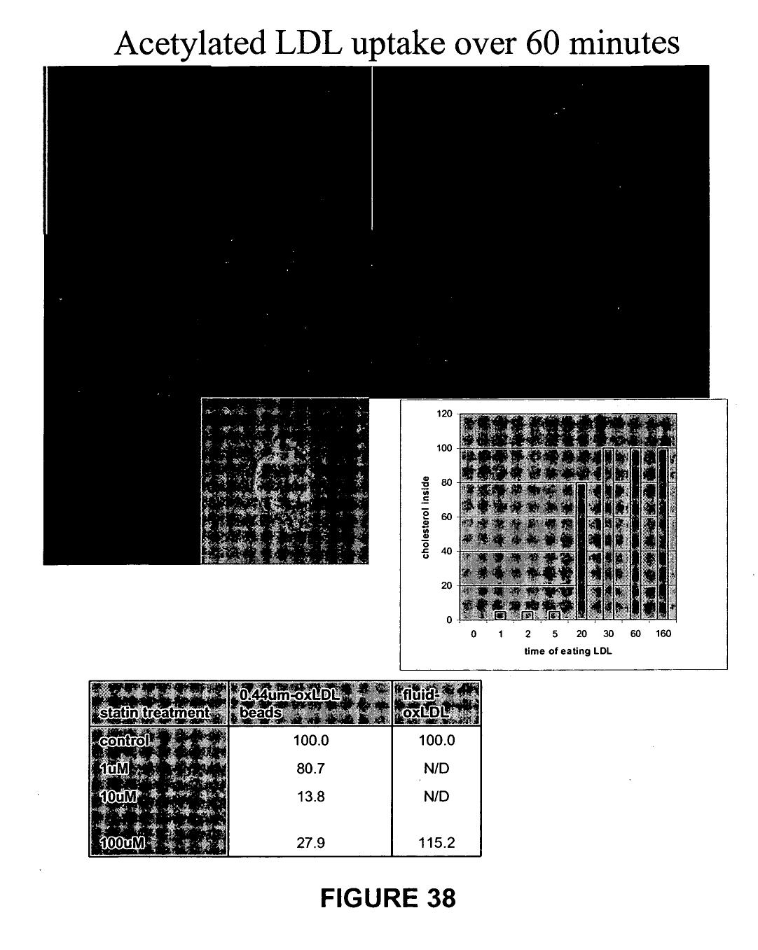

FIG. 38 use of confocal microscopy to assay the accumulation of free fluorescent lipids over time. Statins are the largest selling drugs in the world and their effect of lowering cholesterol has been the basis of the explanation of how they prevent heart attack. However it is not clear in which form the cholesterol that cause heart attacks and stroke is absorbed by macrophage that form foam cells in atherosclerotic plaques leading to heart attack and stroke. We measured the “free” form of fluorescent OX-LDL (bad cholesterol). We measured the uptake of cholesterol by monitoring fluorescence. The effect of lovastatin on the direct uptake of red fluorescent DI-LDL or OX-LDL was measured by red fluorescence confocal microscopy at 594 nm. We found that the statin lovastatin, had no effect on the accumulation of free cholesterol. In contrast we observed that a major effect of statins is to prevent the accumulation of OX-LDL in the form of nano or micro particles.

It has been shown that statins prevent heart attack and stroke and lower cholesterol. The prior art taught that statins only exert their effects directly from the lowering of cholesterol and not from any other mechanism and that lowering of cholesterol alone inhibits Fc mediated phagocytosis of red blood cells. We conclude that statins may prevent the phagocytic engulfment of LDL and modified LDL in the form of fatty streaks in the arteries or large aggregate particles of LDL and other biopolymers or modified particles such as LDL that has been oxidize or bound by proteins. We observed that modified LDL particles were engulfed by macrophages and that statins prevent the engulfment of OX-LDL in the form of larger nano or micro particles, but not free cholesterol.

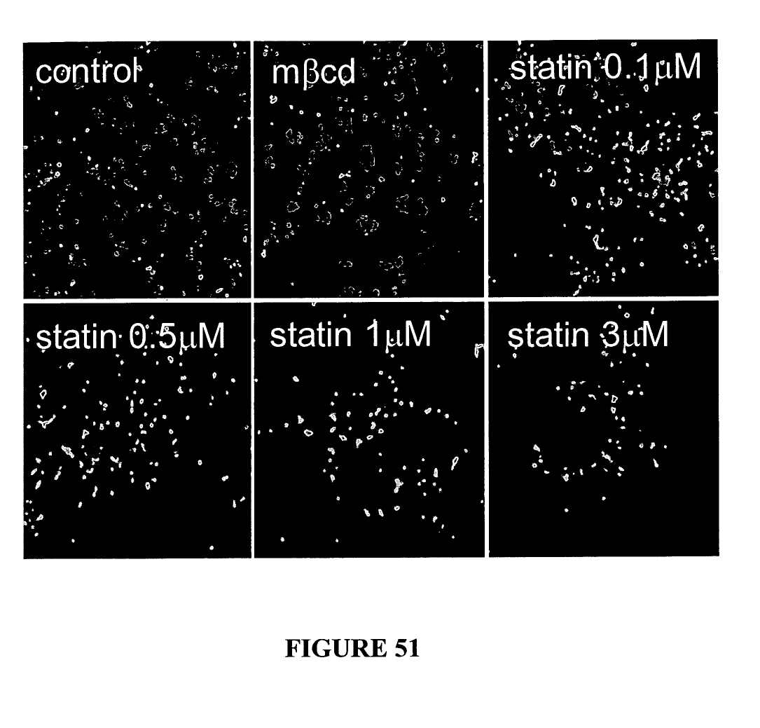

In contrast to the prior art we observed that brief (15 to 30 minute) incubation with methyl beta cyclo dextrin was effective to extract cholesterol but had no effect on the phagocytosis of sheep red blood cells. In contrast to statins, we observed that removing cholesterol from the outer leaflet of the cell membrane with a brief treatment with methyl beta cyclo dextrin had little inhibitory effect on the engulfment of modified particles (FIG. 51), and thus we conclude that one of the major effects of statins may be preventing particle engulfment by leukocytes.

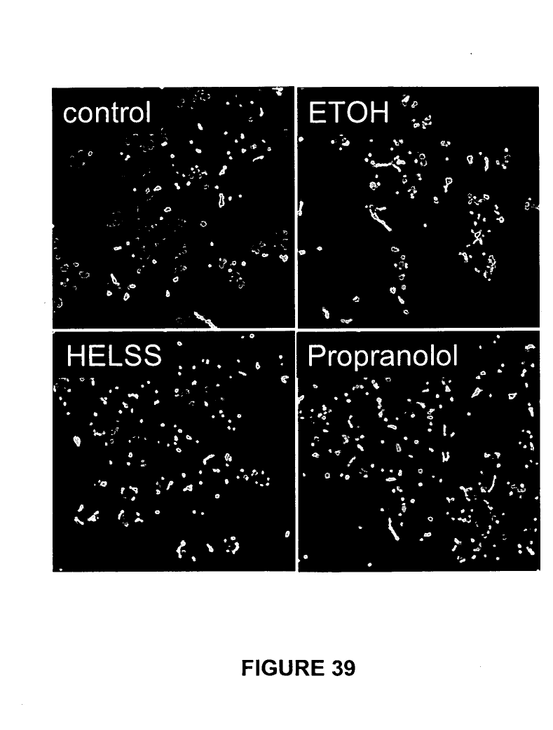

FIG. 39 shows the effect of inhibiting PLD Pathway on Fc Mediated Phagocytosis. The control cells engulf most particles (red). The yellow indicates that Ethanol, propranolol and HELSS prevent particle accumulation. Statins and propranolol have been shown to prevent heart attack. A now well established side effect of ethanol is to prevent heart attack. Thus the common effects of statins, propranolol, and ethanol must be closely linked to the central mechanism that is the most important pharmacological target of heart attack and thus atherosclerosis prevention.

It remains possible that the preventative effect of statins results from their effect to inhibit particle engulfment and foam cell formation by macrophages leading to atherosclerotic plaques. We created a foam cell model and quantitative confocal assays for micro particle engulfment by macrophages. The engulfment of particles by macrophages and the generation of free radicals by leukocytes are the key physiological actions that lead to the generation of foam cells that are the center of atherosclerotic plaques. We made a cellular model system of foam cells or activated leukocytes and examine the effects of moderate levels of alcohol over time on the accumulation of particles and generation of free radicals at the site of particle accumulation compared to statins and propranolol. The model systems will consist of RAW macrophages that engulf hydrophobic polystyrene microparticles with or without coating by ligands or mixtures of ligands such as those found in cellular growth media, or free fluorescently labeled DI-LDL, OX-LDL, Acetyl LDL or other. Live cell confocal microscopy was used to quantify the effects of ethanol, compared to lovastatin and propranolol, on particle engulfment.

The PLD family of enzymes has been previously implicated to control particle engulfment and super oxide generation. Particle engulfment and the oxidative burst have previously been shown to require the function of the phospholipase D (PLD) pathway. Alcohol is the only known inhibitor of PLD and has been previously shown to prevent the phagocytosis of IgG opsonized particles and the oxidative burst in response to mitogenic and bacterial agonists. With the recent understanding that the pathways of scavenger, bacterial and IgG receptors share a common signaling mechanism its seems very likely that ethanol will prevent the accumulation of micro particles and the oxidative burst via the PLD to PAP-1 pathway. The only characterized inhibitor of the PLD enzyme family is alcohol. The protein PLD was detected within the scavenger receptor complex by LC/LC-MS/MS. Here we show that PLD inhibitor ethanol prevented the engulfment of IgG coated beads to a similar extent as HELSS and propranolol. From these results we conclude that the bead-based biology system can be used to find new drug targets associated with an activated receptor and to quantify the effect of drugs and molecular therapeutics in preventing in preventing the activation of the receptor (FIG. 39).

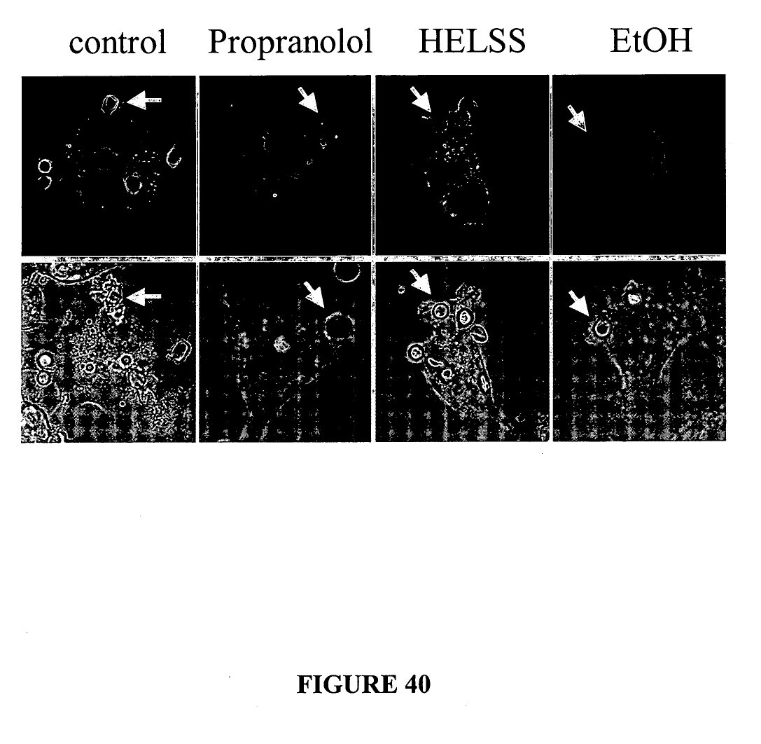

FIG. 40 shows use of PKC/C2-GFP to demonstrate the penetration and efficacy of Propranolol, HELSS and Ethanol (ETOH) to prohibit DAG production at the site of ligand coated bead binding at or near the cell surface (FIG. 39).

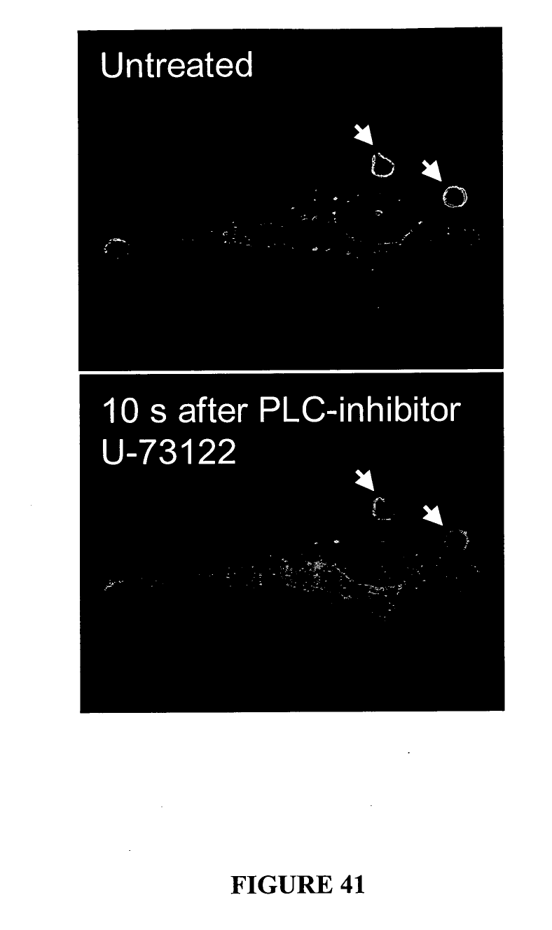

FIG. 41 shows the use of PKC/C2-GFP Domain Measures DAG production at the site of particle engulfment; and to measure the penetrance and efficacy of an potential atherosclerosis drug.

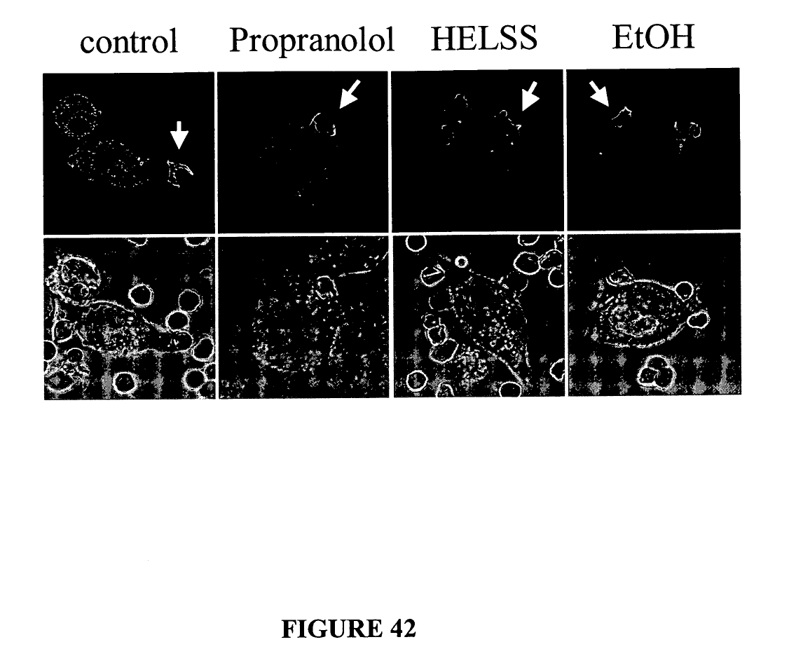

FIG. 42 shows measurement of specificity using Akt/PH-GFP domain to measure PIP3 production at the site of particle engulfment. The fluorescent signal in the presence of propranolol, HELSS and EtOH indicate that these drugs have no side effect on the PI3K pathway to PIP3.

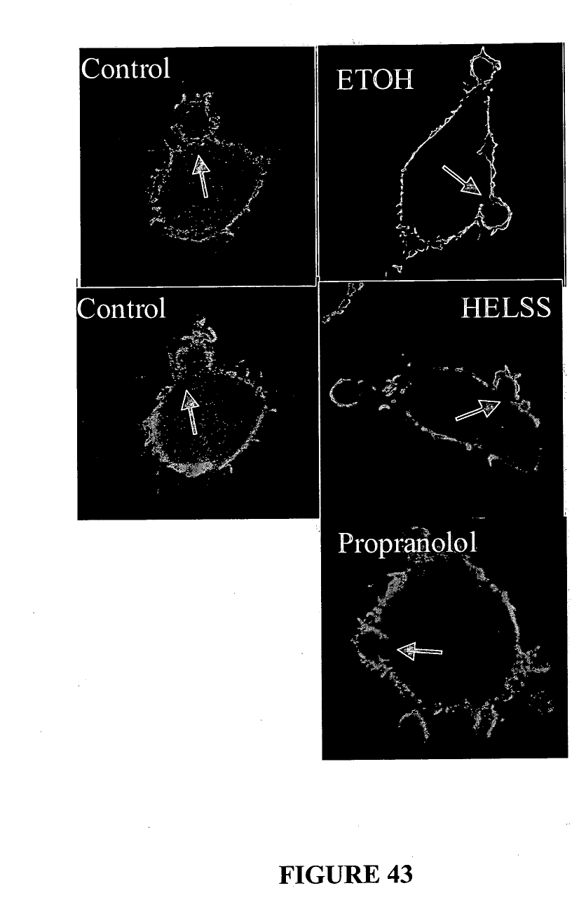

FIG. 43 shows measurement of specificity using the PLC-delta PH domain measures PIP2 catalysis by PLC at the base of the engulfed particle. Note that neither EtOH, HELSS or propranolol interfere with the catalytic action of PLC.

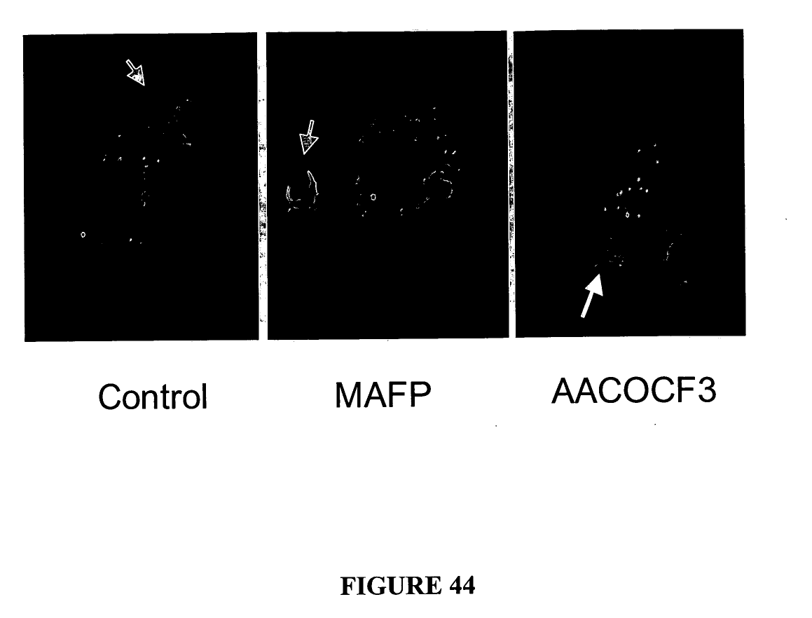

FIG. 44 shows measurement of specificity using the inhibitory effect of HELSS on DAG production as measured by the PKC/C2-GFP is not due to an effect on iPLA2. Neither MAFP nor AACOCF3 prevent DAG production in contrast to the PAP-1 inhibitor HELSS.

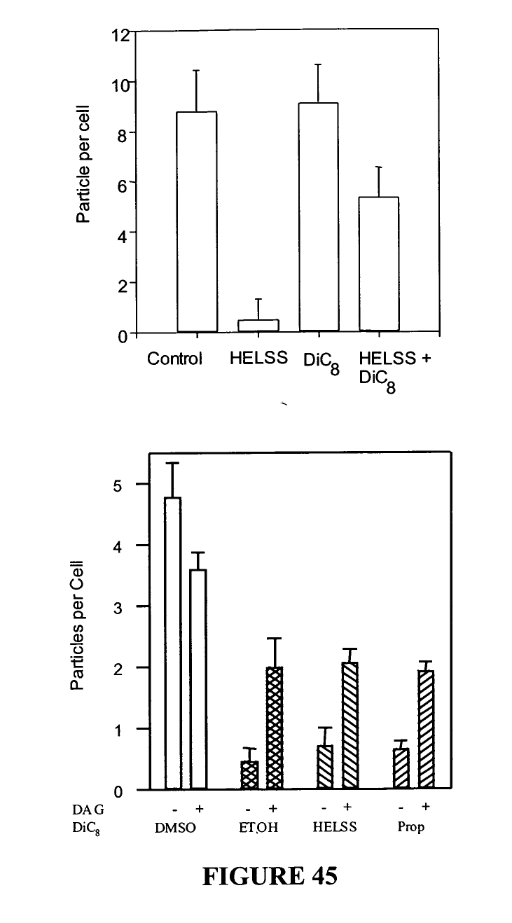

FIG. 45 shows that PLD pathway inhibitors prevent particle engulfment and the effect is reversed by DiC8, the product of the PLD pathway.

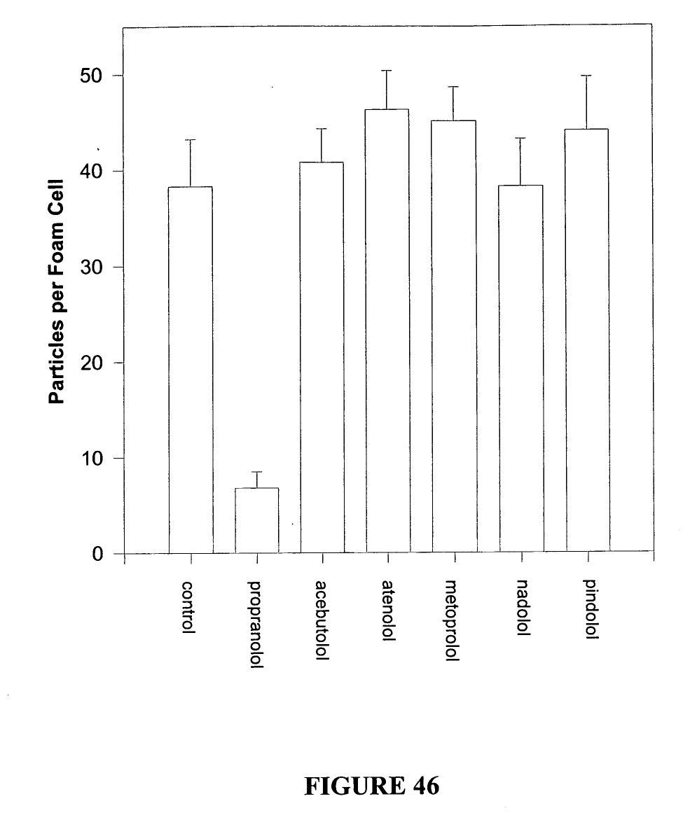

FIG. 46 demonstrates that the effect of propranolol on phagocytic receptor in foam cell formation is not due to its capacity to block the beta-adrenergic receptor. Here we show that PLD to PAP-1 pathway inhibitor propranolol, but not other beta blockade drugs prevented the engulfment of IgG coated beads. Propranolol, but not other effective beta blocker prevent secondary heart attacks. In order to further support a role for the target of HELSS and propranolol, PAP-1 in the prevention of primary heart attacks we determined whether Propanolol, but not other effective beta blockers, prevent the engulfment of particle by macrophages via inhibition PAP-1 and not the beta adrenergic receptor. To this end we demonstrate that the inhibitory effect of propranolol on the particle engulfment does not result from its beta-blocking activity using more a variety of more effective and more modern beta blockers that do not inhibit particle engulfment as a control. We demonstrated that the effect of propranolol to prevent the engulfment of hydrophobic micro-particles by the foam cell model does not result from its beta blocking activity. Propranolol, but not the more modern and effective beta blockers (atenolol, acetbutolol, pindolol, metoprolol, and nadolol), had the largest effect in preventing the engulfment of hydrophobic micro particles via its effect on PAP-1. We observed that propranolol has a much greater effect in preventing the engulfment of microparticles compared to more effective beta blockers. The capacity of the PAP-1 inhibitor propranolol, but not all other effective beta blockers, to prevent the engulfment of hydrophobic micro particles indicates that the inhibition of particle engulfment does not result from beta blockade. The capacity to block particle accumulation via PAP-1 is the key mechanism by which propranolol blocks the formation of foam cells and resulting atherosclerotic plaques (FIG. 46). From these results we conclude that the bead-based biology system can be used to find new drug targets associated with an activated receptor and to quantify the effect of drugs and molecular therapeutics in preventing in preventing the activation of the receptor.

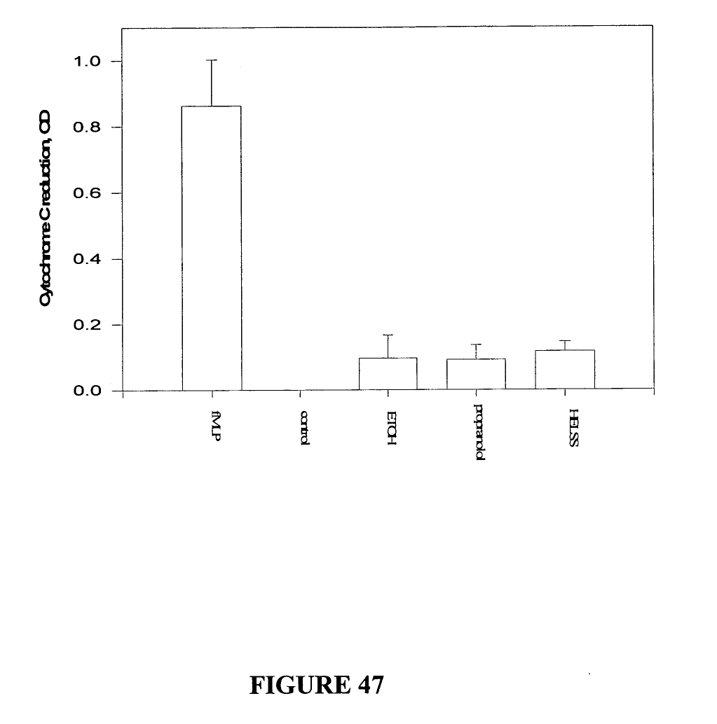

FIG. 47 demonstrates the ability of propranolol to block the oxidative burst which modifies particles that initiate phagocytic receptor foam cell formation. The receptor for the bacterial peptide FMPL has served a general model of the activation of the innate immune system leading to the generation of free radicals that may modify particles. It has been demonstrated that the oxidation of LDL-leads to accumulation of hydrophobic “bad cholesterol” by macrophages perhaps leading to cellular activation, necrosis or apoptosis that might destabilize atherosclerotic plaques leading to heart attack or stroke. It has already been suggested, based only on inhibition by ethanol, that the PLD pathway is required for the oxidative burst by neutrophils. The contention that PLD regulates super oxide formation based on inhibition by ethanol alone is weak. In order to credibly demonstrate that the PLD pathway regulates the NADPH oxidase, evidence from multiple inhibitors of the PLD to PAP-1 pathway including ethanol, HELSS and propranolol were required (FIG. 47). Here we show that the PAP-1 is a drug target inhibited by HELSS and propranolol to block the oxidative burst.

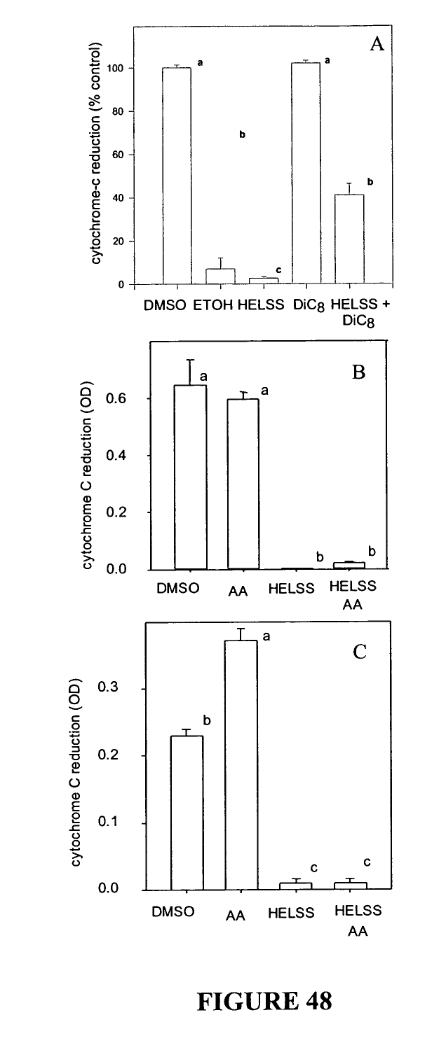

FIG. 48 shows that the product of the PLD pathway, but not the product of the PLA2 pathway rescues oxidative burst. Although the PAP-1 inhibitor HELSS has been shown to inhibit the oxidative burst this result was interpreted to result from its side effect on iPLA2. Further confidence in the role of the PLD pathway could be derived from a partial rescue effect from the inhibitory drugs by adding back a cell permeable form of the product of the pathway, DAG (DiC₈). We demonstrated the role of the PLD pathway in the regulation of the oxidative burst in human neutrophils. The known inhibitors of the PLD/PAP-1 pathway, ethanol, propranolol and HELSS inhibited the fMLP induced oxidative burst in a dose dependant manner and this effect was overcome by the provision of DiC8, a partially cell soluble form of DAG. The provision of exogenous arachidonic acid in add-back experiments of intact and permeabilized cells did not overcome the effect of PAP-1 inhibitors. Human neutrophil leukocytes were isolated from fresh venous blood and pre-treated with the inhibitors of PLD pathway, ethanol, propranolol and HELSS prior to stimulation of the NADPH oxidase using the bacterial peptide fMLP. The effect of these inhibitors on the production of super oxide radicals in neutrophils stimulated with fMLP was measured with the cytochrome C reduction assay using super oxide dismutase treatment as a control to establish the baseline as previously described. We observed that ethanol, propranolol and HELSS all inhibited the oxidative burst in human leukocytes. The exogenous additional of DiC8, but not arachidonic acid (AA), partially recovered the effect of HELSS. All these experiments serve to strongly confirm the previous suggestion that the target of propranolol is magnesium dependant PAP-1 and that this enzyme is the key regulatory event that prevents the generation of super oxide radicals in human leukocytes.

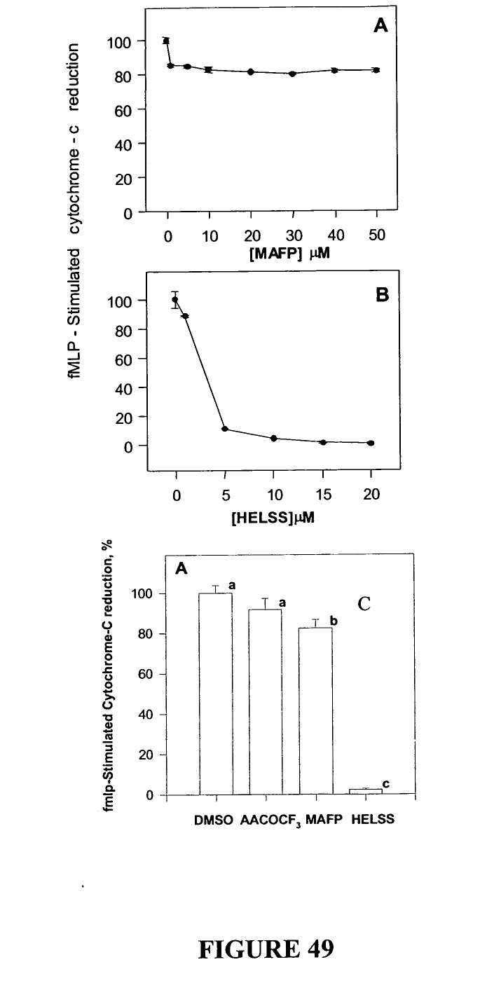

FIG. 49 demonstrates the ability of PLD pathway inhibitors to block the generation of free radicals which modifies particles that initiate phagocytic receptor foam cell formation; the PLD, but not the PLA2 pathway inhibitors act as the blocking agent.

Both PAP-1 inhibitors HELSS and Propranolol block the generation of free radicals and particle engulfment indicating that Mg2+ dependant PAP-1 is a key enzyme in the pathway that to atherosclerosis leading to heart attack and stroke. Although the PAP-1 inhibitor HELSS has been shown to inhibit the oxidative burst this result was interpreted to result from its side effect on iPLA2. Further confidence in the role of the PLD pathway could be derived by controlling for the potential side effect HELSS on the house keeping phospholipid remodeling enzyme iPLA2. Demonstrate the role of Mg2+ dependant PAP-1 pathway in the regulation of the oxidative burst in human neutrophils by showing that the iPLA2 inhibitors MAFP and AACOF3 will not effect the oxidative burst and that the provision of exogenous arachidonic acid in an add-back experiment will not over come the effect of PAP-1 inhibition. The effect of these PLA2 inhibitors on the production of super oxide radicals in neutrophil leukocytes stimulated with fMLP was measured with the cytochrome C reduction assay as previously described. We observed that the control iPLA2 inhibitors MAFP and AACOCF3 had little effect on the oxidative burst (FIG. 49).

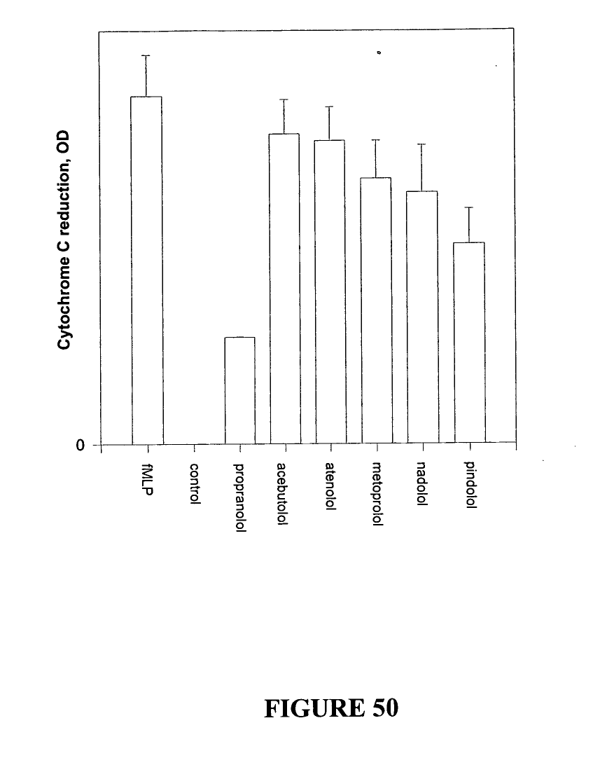

FIG. 50 demonstrates that the effect of propranolol on generating free radicals that initiate phagocytic receptor foam cell formation is not due to its capacity to block the beta-andronergic receptor. Here we show that PLD to PAP-1 pathway inhibitor propranolol, but not other beta blockade drugs prevented the production of free radical oxygen by human leukocytes. Propranolol, but not other effective beta blocker prevent secondary heart attacks. In order to further support a role for the target of HELSS and propranolol, PAP-1 in the prevention of primary heart attacks we determined whether Propanolol, but not other effective beta blockers, prevent the generation of super oxide radicals. To this end we demonstrate that the inhibitory effect of propranolol on the oxidative burst does not result from its beta-blocking activity using more a variety of more effective and more modern beta blockers that do not inhibit particle engulfment as a control. We demonstrated that the effect of propranolol to prevent the engulfment of hydrophobic micro-particles by the foam cell model does not result from its beta blocking activity. Propranolol, but not the more modern and effective beta blockers (atenolol, acetbutolol, pindolol, metoprolol, and nadolol), had the largest effect in preventing the generation of free radicals that modify lipid particles increasing their engulfment via its effect on PAP-1. We observed that propranolol had a much greater effect in preventing the generation of free radicals. The capacity of the PAP-1 inhibitor propranolol, but not all other effective beta blockers, to prevent the engulfment of hydrophobic micro particles indicates that the inhibition of free radical production does not result from beta blockade. The capacity to block free radical production via PAP-1 is a key mechanism by which propranolol blocks the formation of foam cells and resulting atherosclerotic plaques. The highly effective beta blockers that served as controls were not as effective as the PAP-1 inhibitor propranolol at preventing the generation of super oxide radicals.

FIG. 51 demonstrates the effect of statins on phagocytic engulfment of particles. Control and cholesterol scavenger MBC still engulf particles (red). Statins prevent engulfment (no red) and particles are stranded outside (yellow). Statins are the largest selling drugs in the world and their effect of lowering cholesterol has been the basis of the explanation of how they prevent heart attack. Methyl-beta-cyclo-dextrin is a highly effective cholesterol scavenger and can reduce cellular cholesterol content below that produced by statins. We show that lovastatin at doses as low as 100 nM, but not Methyl Beta Cyclo Dextran (MBCD) at 5 mM, directly prevent the engulfment of these particles by cultured macrophage leukocytes.

We sought to determine if statin drugs such as lovastatin prevent the formation of foam cells via an effect on cellular cholesterol levels. If statins drugs prevent the engulfment of modified microparticles by leukocytes or macrophages that lead to the formation of foam cells via cholesterol lowering then the effective cholesterol scavenger agent MBCD should show similar effects. RAW 264.7 macrophages were cultured in alpha MEM with 5% fetal calf serum as described. The effects of lovastatin on IgG and LDL coated micro-particle engulfment at 0, 50 nM, 100 nM, 500 nM, 1 μM and higher was tested.

External particles were stained green with secondary anti rabbit FITC and then cells were permeablized and all particles stained red with secondary anti rabbit CY3. Thus engulfed particles appear red while external particles appear yellow. Beads within the cells carrying only the red signal (engulfed) and not also the green signal {red+green=yellow} (outside) were quantified for accumulation assays using both total fluorescence in the green channel and by counting individual beads in three fields on three independent cover slips for each concentration. MBCD has no effect on particle engulfment. We conclude that the effect of statins to prevent the formation of foam cells in a model of atherosclerotic plaques does not result from its direct effect on cellular cholesterol

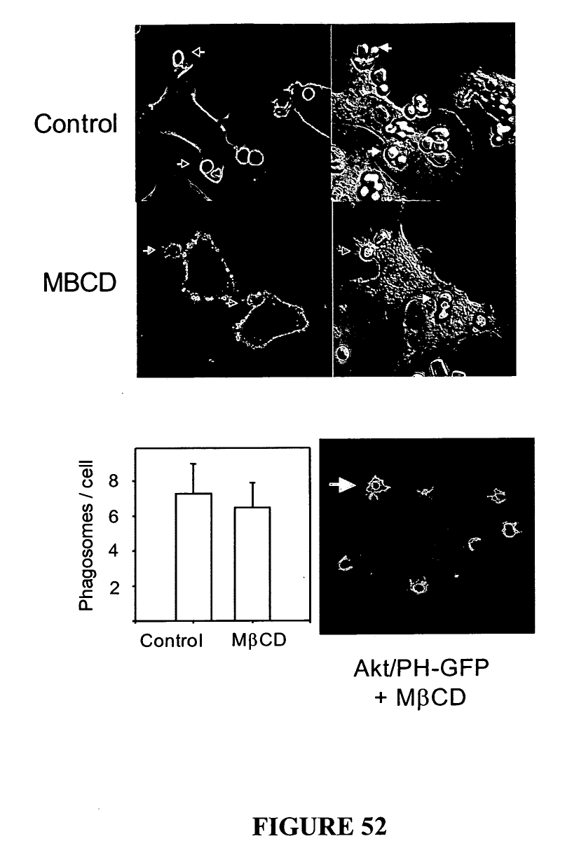

FIG. 52, top panel, shows Filipin staining of cholesterol at the site of IgG coated particles in control RAW cells and where cholesterol was extracted with MBCD, and (Bottom): shows the effect of MBCD on engulfment and the accumulation of PIP3 at IgG coated particles. Cholesterol levels were quantified by the esterase assay, oil red- and filipin-staining. Macrophages engulfed particles very efficiently in the presence of MBCD but failed to accumulate particles in the presence of statins. Methyl-beta-cyclo-dextrin is a highly effective cholesterol scavenger as measured by filipin staining yet has no effect on particle accumulation indicating that the effect of statins to prevent the engulfment particles by foam cells is not directly dependant on their effect on cholesterol.

Cholesterol was not required for the generation of PIP3 at the site of particle accumulation or PI3K signaling as measured by AKT/PH-GFP in cell with and without MBCD treatment indicating that lipid rafts are not responsible for the localization PIP3 to the membrane at the site of receptor activation. The capacity of the cellular model of foam cells in atherosclerotic plaques to engulf particles was not dependant on the presence or absence of cholesterol (FIG. 51-57). Cholesterol removal by the biopolymer modification material methyl beta cyclo dextrin did not prevent Phosphatidyl Inositol 3 Kinase (PI3K) signaling reflected by Phosphatidyl Inositol 3,4,5-tri Phosphate (PIP3) production as measured by the PIP3 binding domain of AKT fused to Green Fluorescence Protein (GFP). Removing cholesterol with MBCD had no effect on particle accumulation. Lovastatin's capacity to prevent particle engulfment apparently did not result from its effect on cholesterol levels. Statin also prevent the formation of many isoprenoids other than the C30 cholesterol including the geranyl and farnesyl isoprenoids that anchor small G proteins and have been linked to the function of the PLD pathway. Statins directly prevent the engulfment of microscopic particles by macrophages and that lead to the formation of foam cells. The capacity of statins to directly prevent the accumulation of hydrophobic micro particles by foams cells may be the major mechanisms contributing to the capacity of statins to prevent atherosclerosis leading to heart attack and stroke (FIG. 52).

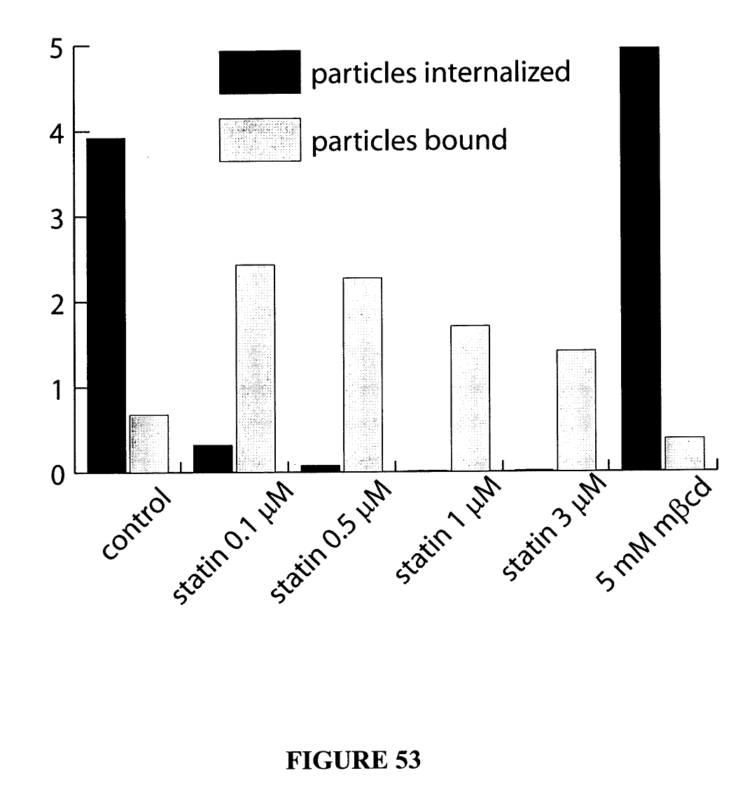

FIG. 53 demonstrates quantification of the effect of cholesterol lowering drugs on macrophage mediated model of foam cell formation.

If statin drugs prevent the accumulation of hydrophobic microparticles by macrophages that lead to the formation of foam cells via cholesterol lowering then the effective cholesterol scavenger agent MBCD should show similar effects.

RAW 264.7 macrophages were cultured in alpha MEM with 5% fetal calf serum as described. The effects of lovastatin on IgG and LDL coated micro-particle engulfment at 0, 50 nM, 100 nM, 500 nM, 1 mM and higher was tested. The accumulation of free DI-LDL was quantified directly by its red fluorescence. External particles were stained green with secondary anti rabbit FITC and then cells were permeablized and all particles stained red with secondary anti rabbit CY3. Thus engulfed particles appear red while external particles appear yellow. Beads within the cells carrying only the red signal (engulfed) and not also the green signal {red+green=yellow} (outside) were quantified for accumulation assays using both total fluorescence in the green channel and by counting individual beads in three fields on three independent cover slips for each concentration. The effect of MBCD and lovastatin on the direct uptake of red fluorescent DI-LDL (Intracel, Frederick, Md.) was measured by fluorescence confocal microscopy at 594 nM (FIG. 53).

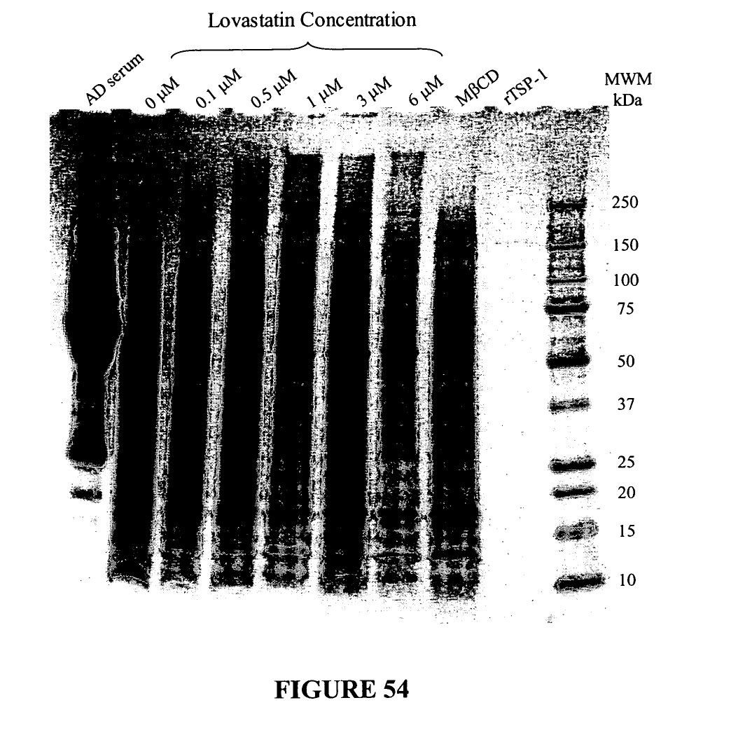

FIG. 54 demonstrates the biochemical measurement of membrane protein from RAW macrophages treated with a drug. Statins and propranolol have been shown to prevent heart attack. A now well established side effect of ethanol is to prevent heart attack. Thus the common effects of statins, propranolol, and ethanol must be closely linked to the central mechanism that is the most important pharmacological target of heart attack and thus atherosclerosis prevention. It remains possible that the preventative effect of statins results from their effect to inhibit particle engulfment and foam cell formation by macrophages leading to atherosclerotic plaques. We created a foam cell model and quantitative confocal assays for micro particle engulfment by macrophages. The engulfment of particles by macrophages and the generation of free radicals by leukocytes are the key physiological actions that lead to the generation of foam cells that are the center of atherosclerotic plaques. We have made a cellular model system of leukocytes or activated leukocytes or leukocytes treated over time to examine the effects drugs and biopolymer modification materials over time on the activation of receptors at the site of modified or ligand coated particles. The drugs used in this system to examine the kinetics of receptor function at modified particles may result in changes to the cell including changes in gene expression at the level of DNA transcription, RNA production, accumulation or post-transcription processing, mRNA production accumulation or expression all of which may also alter protein expression. We examined the use of these model systems with and without beads as a system to screen the effects of drugs that are known to prevent heart attack and stroke in model of leukocyte cells within atherosclerotic plaques. We observe that drugs that effect receptor associated signaling may also alter gene, RNA or ultimately protein expression in cells resulting in changes in protein levels. Proteins that change levels in response to a drug may be themselves drug target proteins. The drug lovastatin was observed to alter levels of proteins in the membranes of the leukocytes, RAW macrophages (FIG. 54).

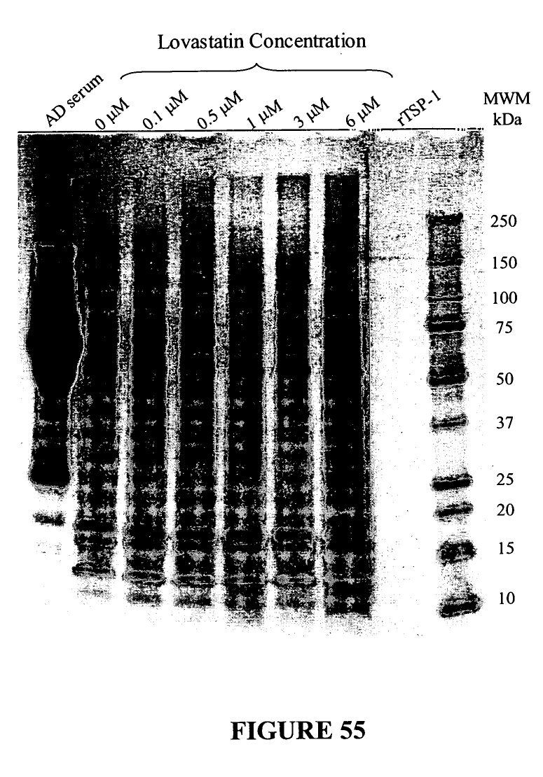

FIG. 55 demonstrates the biochemical measurement of matrix proteins from RAW macrophages treated with a drug. The drug lovastatin was observed to alter levels of proteins in the extracellular matrix of the leukocytes, RAW macrophages (FIG. 55).

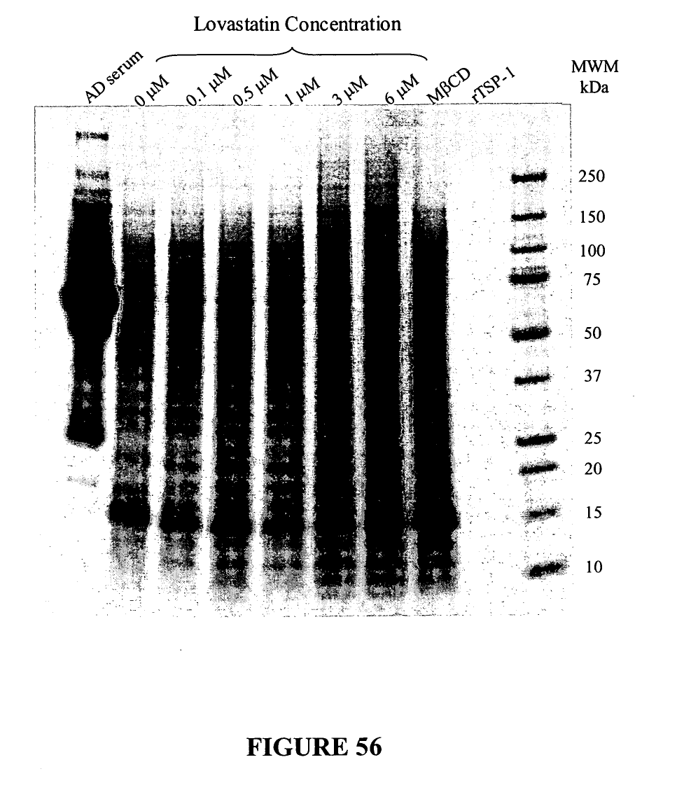

FIG. 56 demonstrates the biochemical measurement of secreted proteins from RAW macrophages treated with a drug. The drug lovastatin was observed to alter levels of proteins in the secretions of the leukocytes, RAW macrophages (FIG. 56).

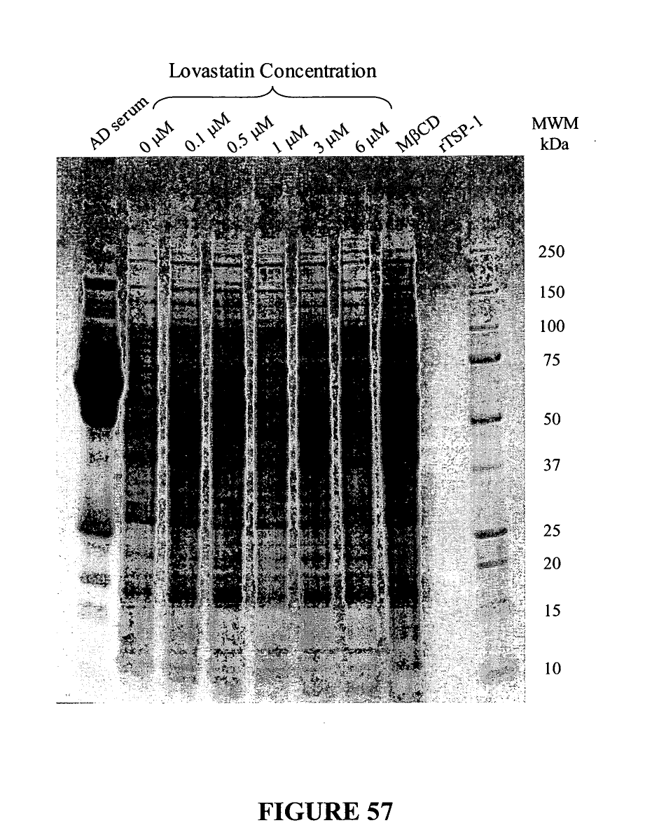

FIG. 57 demonstrates the biochemical measurement of cytosolic proteins from RAW macrophages treated with a drug. The drug lovastatin was observed to alter levels of proteins in the membranes of the leukocytes, RAW macrophages (FIG. 57).

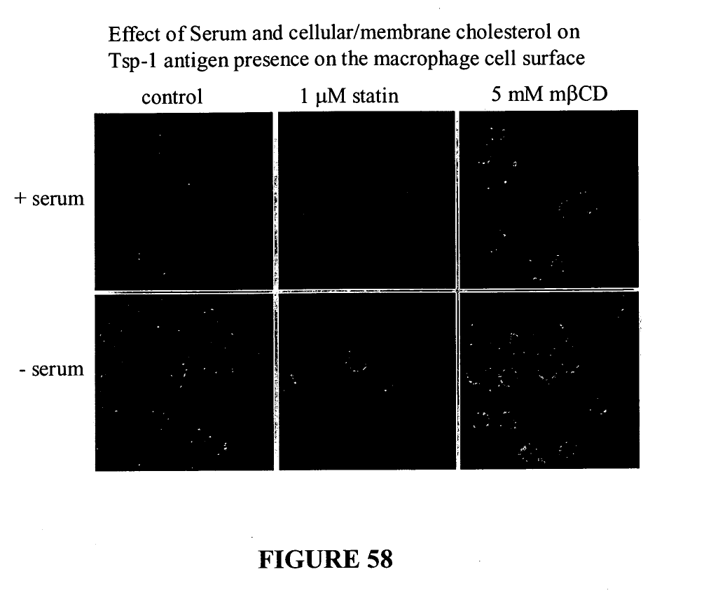

FIG. 58 shows the effect of statin on the surface expression of thrombospondin (TSP). Thrombospondin is a protein that is known to bind apolipoprotein and lipid receptors expressed on the surface of cells including the scavenger receptor class B multi ligand receptor CD36 associated with atherosclerosis and Alzheimer's. The proteins associated with particle engulfment were determined by capturing the intact signaling receptor pathway using the ligand coated bead method and by identifying all the proteins by LC-MS/MS, which revealed the presence of Thrombospondin.

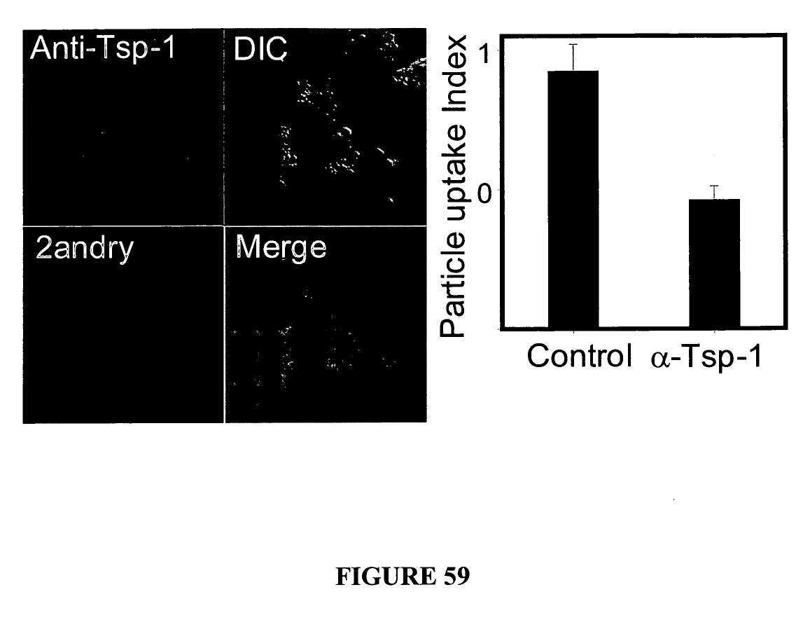

Treating cells with lovastatin lowered the expression of Thrombospondin on the cell surface of the macrophages. As previously shown lovastatin also reduced the capacity of RAW macrophages to engulf particles. Thrombospondin was observed on the surface of red blood cells that were engulfed by RAW macrophages. The therapeutic molecule lovastatin that effects the expression or function of thrombospondin results in a decrease in particle engulfment by foam cells. Together with the role of thrombospondin in the engulfment of modified particles, the reduction of surface expression of thrombospondin that accompanies lovastatin treatment indicates that thrombospondin and anti thrombospondin antibodies are both biopolymer modulation materials that may effect particle engulfment by leukocytes or foam cells and that thrombospondin is a therapeutic target in foam cell formation in atherosclerotic plaques leading to heart attack and stroke. FIG. 59 demonstrates quantification of the inhibitory effect of anti-Thrombospondin 1 antibodies on particle engulfment.

If thrombospondin plays a direct functional role in facilitating particle engulfment by macrophages then specific reagents should alter thrombospondin expression or the function of surface receptor proteins. The Role of Thrombospondin in the engulfment of modified particles was demonstrated using the specific affinity antibodies against thrombospondin in the RAW macrophage foam cell model system. Pre-treating the model cells system with an anti TSP antibody markedly reduced particle accumulation (FIG. 59).

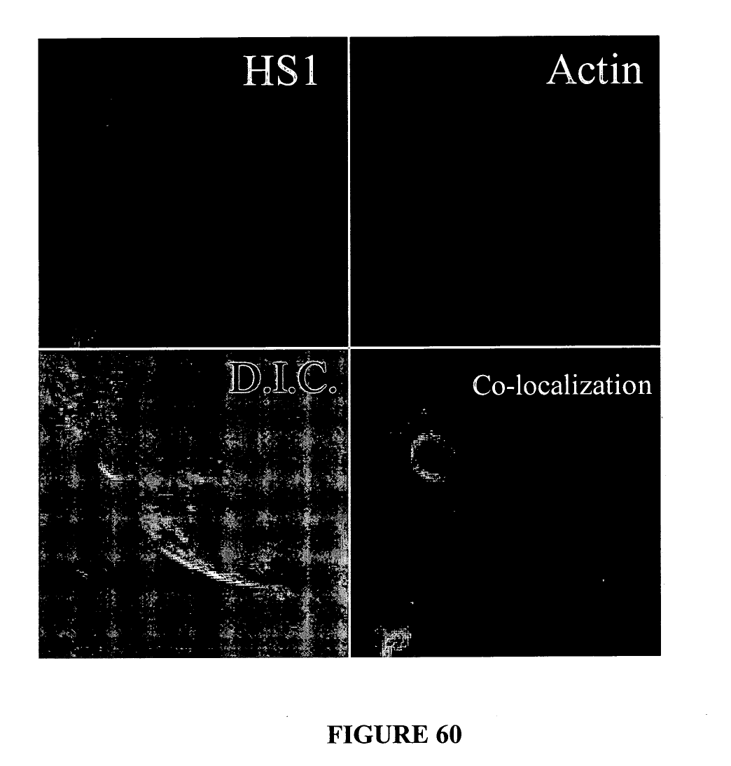

FIG. 60 shows use of ligand covered beads to demonstrate protein-protein interaction of Actin and HS1 on or near the cell surface using confocal microscopy.

Protein interactions and protein complex interactions may be assayed by confocal microscopic measurements at the site of the ligand coated beads. The protein HS1 was discovered on modified or ligand coated microbeads bound to surface receptors on the leukocyte RAW macrophage. HS1 is a protein that has been hypothesized to interact with Actin. Here we used the ligand coated bead system to demonstrate the protein-protein interactions between HS1 and Actin in situ at the site of activated receptors at or near the cell surface.

If two proteins interact or form part of the same activated receptor complex or receptor pathway then the two proteins should both accumulate at the same type of ligand coated or uncoated bead and at the same time and in the same space. We examined the distribution of Actin and HS1 at the site of ligand coated beads versus elsewhere in the cells. We observed that both HS1 and Actin both showed accumulation at the same site where the ligand coated bead was in contact with the surface membrane of the cell at the same time. Thus the use of modified particles such as ligand coated beads may be used to detect or confirm protein-protein interactions or interaction between other biopolymers or biopolymer modulating materials. (FIG. 60).

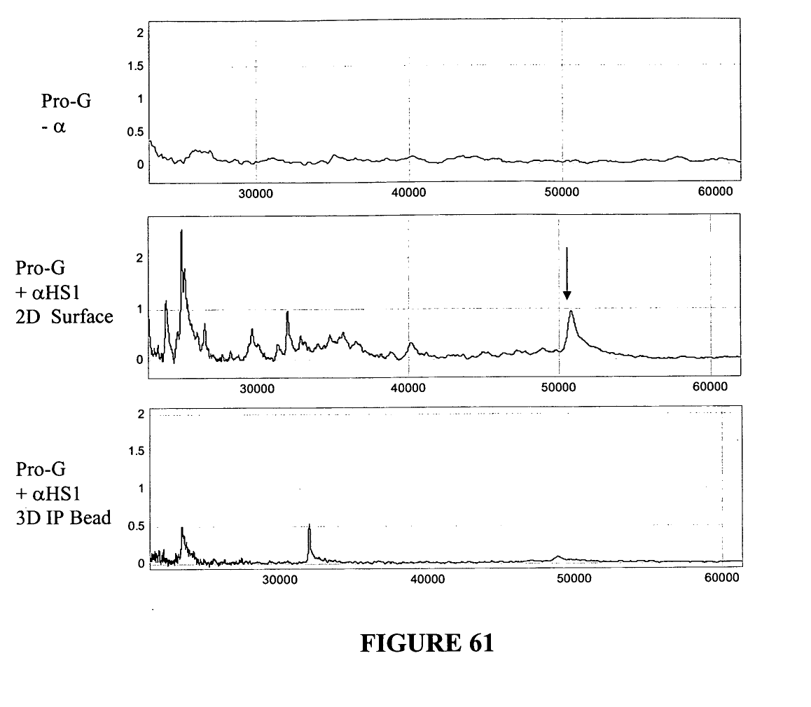

FIG. 61 shows the use of a 2D surface to characterize to characterize protein-protein interactions of HS1 by mass spectrometry.

Protein-ligand Interaction on a modified surface 2 dimensional or 3 dimensional surface. In the instant invention a bead may be a 2 dimensional or three dimensional object. We have shown in FIGS. 1 to 60 that beads may be used to effect ligand receptor interaction on the surface of live cells. The interior of a capillary such as a silica capillary for LC-MS/MS is essential a curved 2 dimensional surface. Capillaries may be packed or filled with chromatography resins which are in essence 3 dimensional particles that may be penetrated. Proteins may interact with other proteins or macromolecular complexes or metabolite or small molecules or polypeptides collectively termed ligands. The ligands that interact with proteins can be determined using affinity chromatography. The affinity chromatography is typically performed using 3 dimensional beads However 3 dimensional beads have a large volume and surface area for non-specific interactions and typically capture far more proteins than are required for MS analysis. Hence MS analysis might be performed with the much smaller amount of analyte captured on 2 dimensional surfaces but the 2 dimensional surface may have a much higher concentration of the specific ligand per unit area but requires significantly less total proteins or affinity capture reagent while achieving a result that is also sensitive for the specifically-binding ligands. We demonstrated that protein ligands interactions can be effected on 2 dimensional surfaces other than MALDI or SELDI targets and that the resulting interacting ligands or protein complexes can be eluted and subsequently analyzed by mass spectrometry. A normal phase silica surface washed in HCl, water and then ethanol before interacting with polylysine. The polylysine was treated with paraformaldehyde and reacted with protein G. The surface was then reacted with anti HS1 antibody, quenched with glycine and then equilibrated with PBS. A crude homogenate of RAW macrophages was then interacted with the normal phase surface, washed 3 times in PBS and followed by three washed in water and elution in 50% acetonitrile with 0.2% formic acid that was spotted onto a metal MALDI target and analyzed by MALDI TOF. We observed that a protein matching the mass of HS1 of about 51 kD was detected on the normal phase interacting surface in addition to other ligands of a variety of molecular masses. We also used protein G chromatography beads on which the protein ligand interaction was performed prior to elution of the ligands and subsequent collection over C18 chromatography prior to analysis by mass spectrometer. This embodiment of protein-ligand interaction of the permit the detection and analysis of ligands that bind protein by mass spectrometry. We conclude that proteins ligand interactions can be detected on a flat or curved 2 dimensional surface prior to elution and analysis by mass spectrometry with or without collection of the ligands by chromatography (FIG. 61).

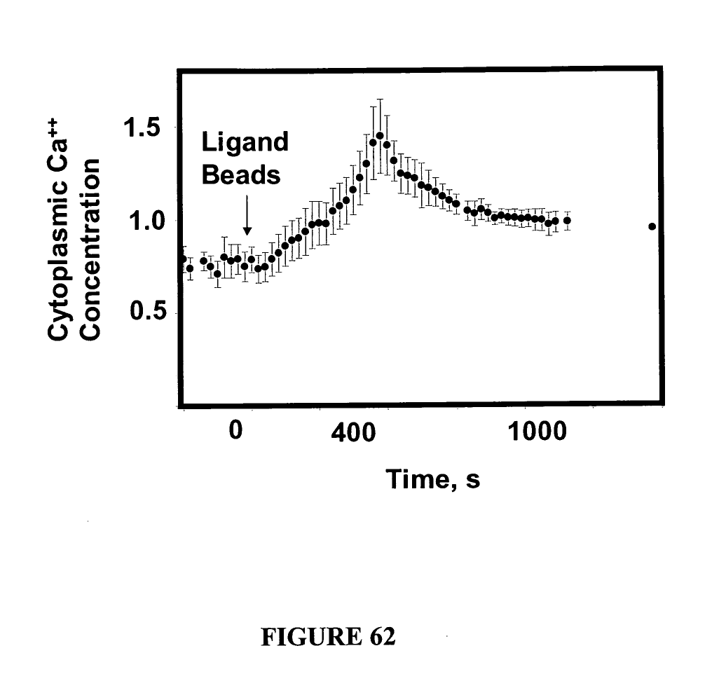

FIG. 62 shows the use of ligand coated beads to screen the function of an ion channel. The ligand coated bead system can also be used to measure the kinetic of receptor activation in response to ligands. The ligand coated beaded system can also be used to determine changes in the levels of calcium, phosphorylated lipids or other signaling events over time. Stimulating RAW macrophages with IgG coated beads produced a transient change in cellular free calcium.

Similarly stimulated RAW cells with ligand coated beads produced a transient increase in the accumulation of the PIP3 binding domain from AKT fused to GFP at the site of activated receptor where the ligand coated bead contacts the surface membrane of the cell. Stimulation of the RAW macrophage foam cell model system with receptor associated g protein stimulatory drug ALF4 (with peroxy vanadate serving as a positive control) was monitored by western blots problem with an anti phosphotyrosine antibody. The cells were disrupted with a mortar and pestle, or sonication, or detergents or a French press or other methods. The cellular contents were then separated into different fractions based on buoyant density by differential centrifugation.

Levels of calcium, lipid phosphorylation and protein phosphorylation could all be monitored with respect to time of cellular activation by ligand coated beads or other stimulatory treatments (FIG. 62-68). The RAW macrophage model foam cells system alone or in combination with the bead based biology system can be used to characterize the kinetics of receptor or cellular activation in terms of second messengers such as calcium, lipid phosphorylation and protein phosphorylation with respect to time.

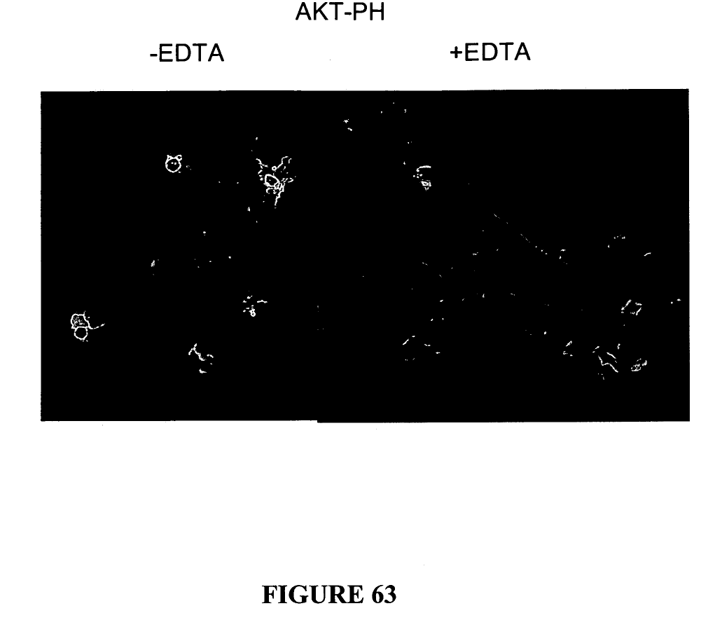

FIG. 63 shows the use of protein binding domain to view ionic signaling at the site of ligand coated beads. Similarly stimulated RAW cells with ligand coated beads produced a transient increase in the accumulation of the PIP3 binding domain from AKT fused to GFP at the site of activated receptor where the ligand coated bead contacts the surface membrane of the cell (FIG. 63).

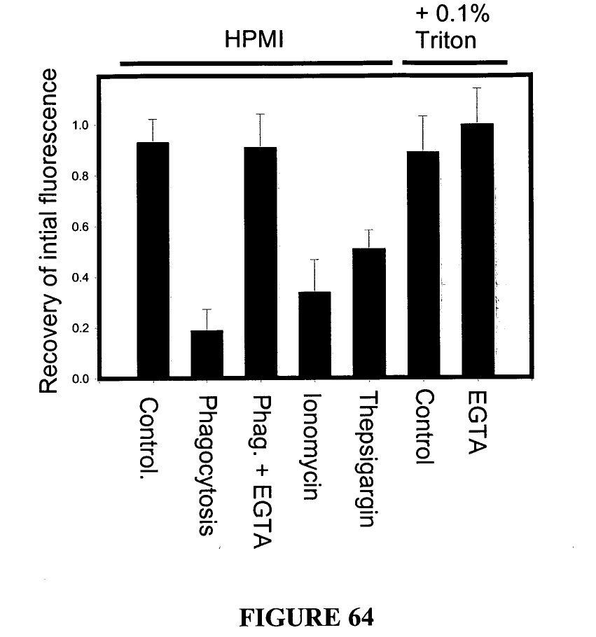

FIG. 64 shows the use of ligand bead/Confocal assay system to view drug effect on ion levels and their downstream effects on the mobility of receptor associated proteins at or near the site of modified particles or ligand coated beads at or near the cell surface.

For example we screened the effect of drugs that alter calcium levels in cells to measure their effect on the mobility of the SRC class proteins LYN's N terminus fused to GFP as measured by FRAP analysis.

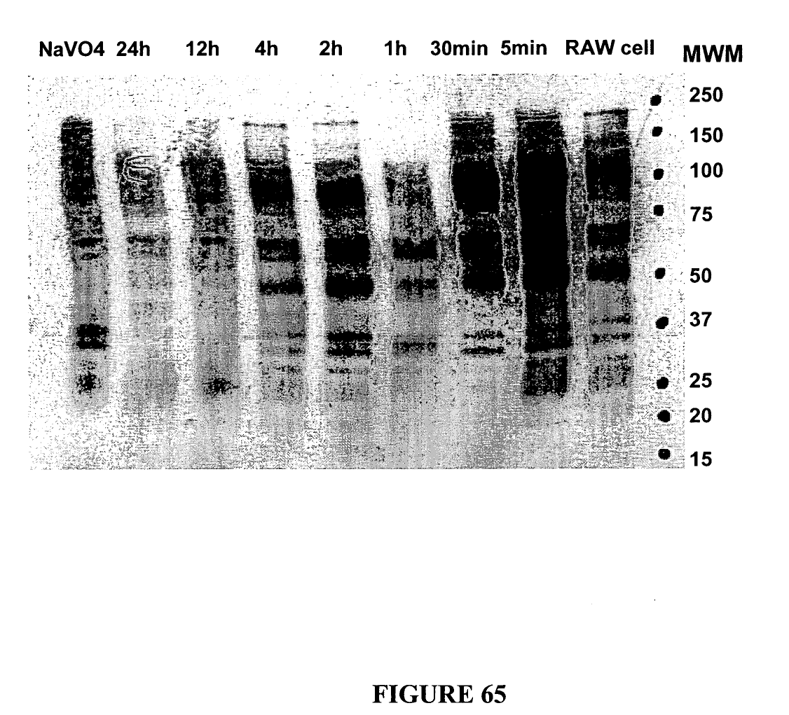

FIG. 65 shows the use of RAW macrophages to view receptor associated protein phosphorylation in the cell matrix. RAW cells stimulated with ligand coated beads produced a transient increase in the accumulation of the PIP3 binding domain from AKT fused to GFP at the site of activated receptor where the ligand coated bead contacts the surface membrane of the cell (FIG. 27, 31, 33). Receptor associated signal proteins can also be stimulated or inhibited with drugs. Stimulation of the RAW macrophage foam cell model system with receptor associated G protein stimulatory drug Alf4 (with peroxy vanadate serving as a positive control) was monitored by western blots problem with an anti phosphotyrosine antibody. The cells were disrupted with a mortar and pestle, or sonication, or detergents or a French press or other methods. The cellular contents were then separated into different fractions based on buoyant density by differential centrifugation. Protein phosphorylation in the leukocyte model of foam cell formation could monitored with respect to time or cellular activation by biochemical means such as immuno staining with western blots. The RAW leukocyte model foam cells system alone or in combination with the bead based biology system can be used to characterize the kinetics of receptor or cellular activation in terms of second messengers such as lipid and protein phosphorylation with respect to drug treatment or time in the matrix of leukocytes.

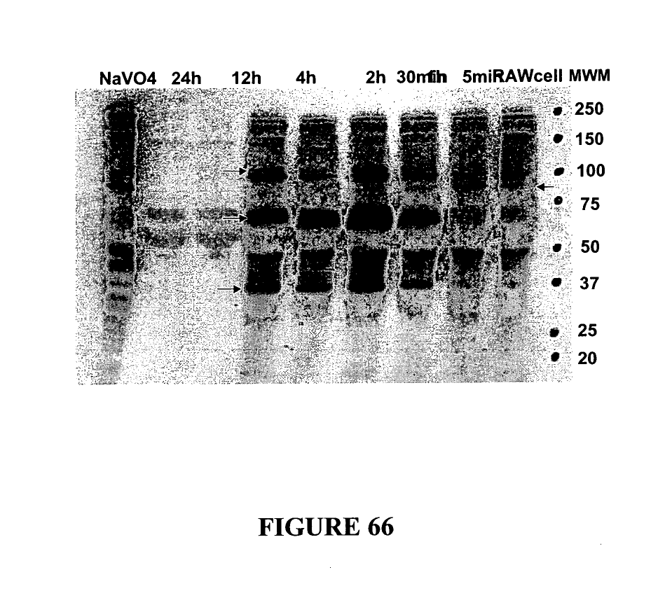

FIG. 66 shows the use of RAW macrophages to view receptor associated protein phosphorylation in macrophages. The RAW leukocyte model foam cells system alone or in combination with the bead based biology system can be used to characterize the kinetics of receptor or cellular activation in terms of second messengers such as lipid and protein phosphorylation with respect to drug treatment or time in the cytosol of leukocytes.

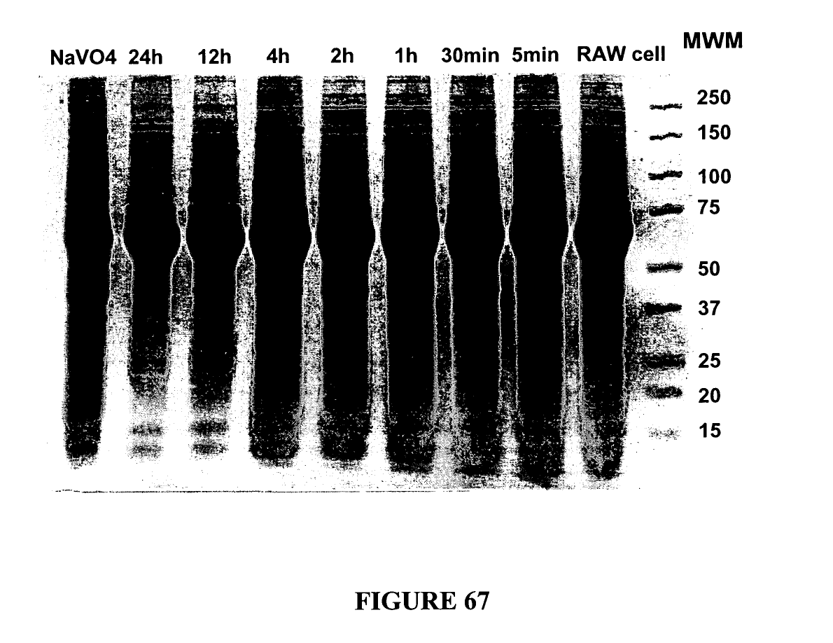

FIG. 67 shows the use of biochemical analysis of receptor associated protein activation in macrophages. The drugs used in the leukocyte model system to examine the kinetics of receptor function at modified particles may result in changes to the cell including changes in gene expression at the level of DNA transcription, RNA production, accumulation or post-transcription processing, mRNA production accumulation or expression all of which may also alter protein expression. We examined the use of these model systems with and without beads as a system to screen the effects of drugs that are known to effect receptor response at or near the cell surface or interior of the cells. We observe that drugs that effect receptor associated signaling may also alter gene, RNA or ultimately protein expression in cells resulting in changes in protein levels. Proteins that change levels in response to a drug may be themselves drug target proteins. The receptor associated G protein stimulatory drug AlF4 was observed to alter levels of proteins in the membranes of the leukocytes, RAW macrophages. The RAW leukocyte model foam cells system alone or in combination with the bead based biology system can be used to characterize the effects of receptor or cellular activation in terms of second messengers such as lipid and protein phosphorylation with respect to drug treatment or time in the cytosol of leukocytes (FIG. 67).

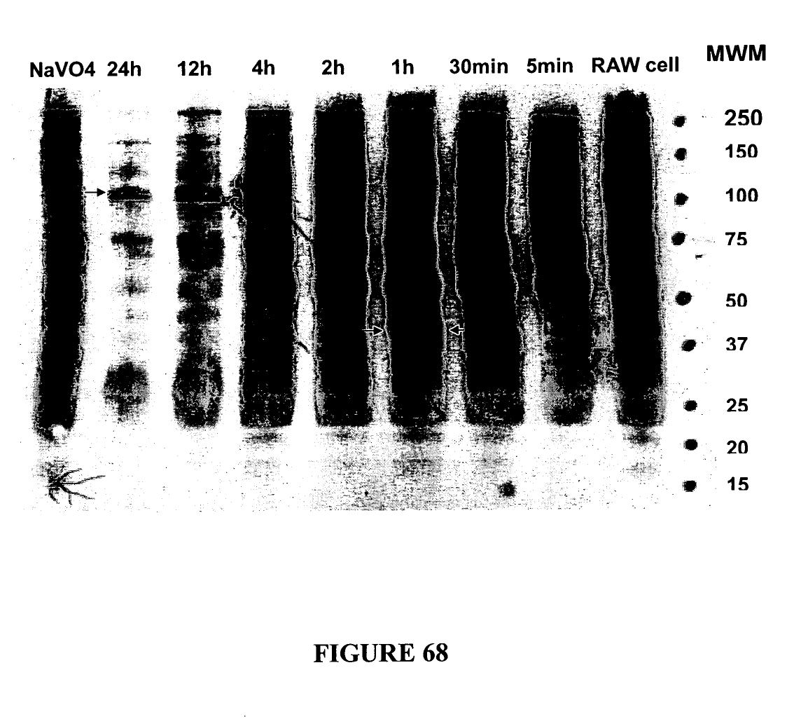

FIG. 68 shows the use of RAW macrophages to view receptor associated protein phosphorylation in macrophages. The RAW leukocyte model foam cells system alone or in combination with the bead based biology system can be used to characterize the kinetics of receptor or cellular activation in terms of second messengers such as lipid and protein phosphorylation with respect to drug treatment or time in the membrane of leukocytes.

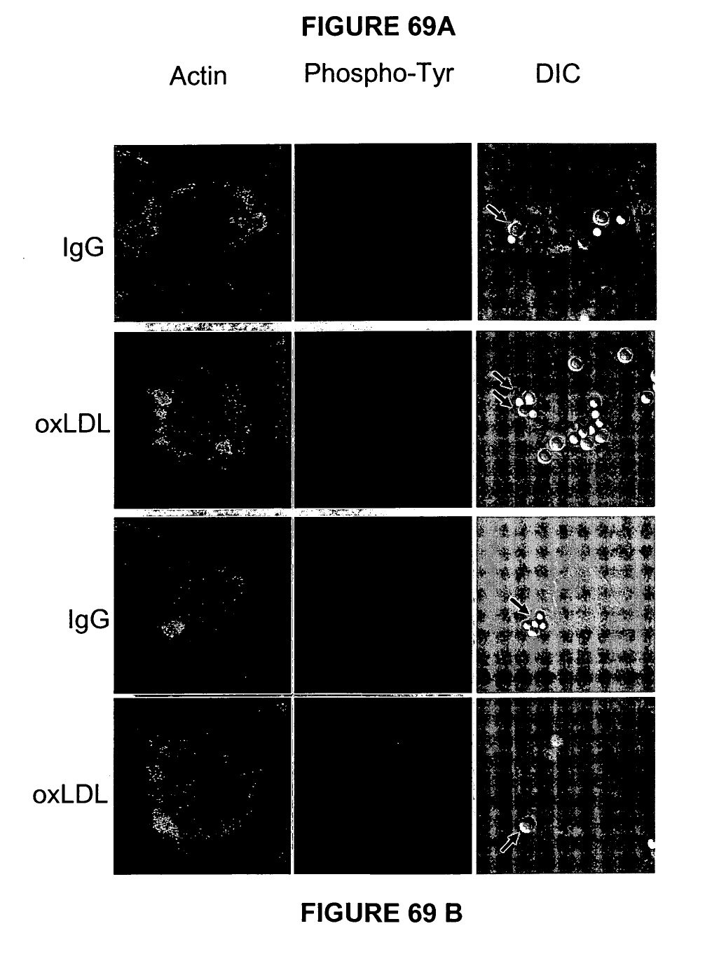

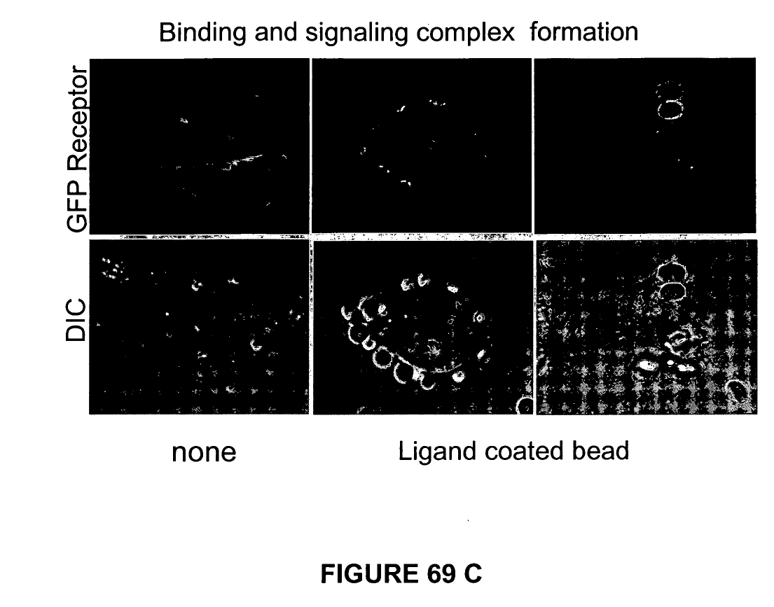

FIG. 69. Illustrates J774, CHO cells expressing the Fc receptor and RAW 264.7 leukocytes binding IgG and oxLDL coated 2 um beads at the cell surface. Associated Actin (green) and phospho-Tyrosine accumulation at the vicinity of ligand-coated and receptor associated complex formation is shown;

Model cells may be created to contain receptors or receptor associated proteins to test their function and mechanism of action. For example the Fc receptors was transfected into the CHO cell line conferring on the CHO cells the ability to engulf particles in a manner similar to leukocytes (FIG. 69).

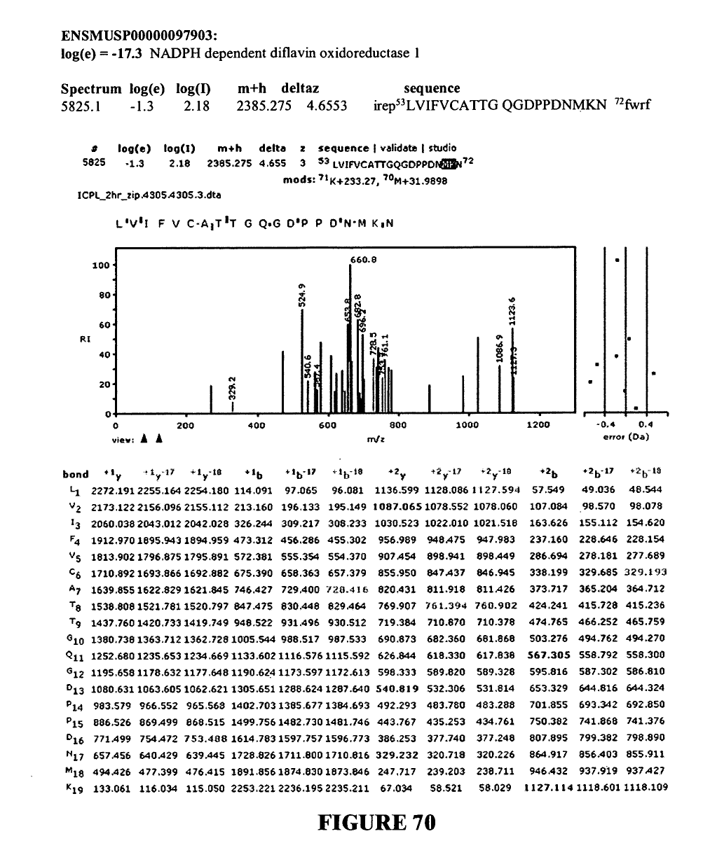

FIG. 70. Mascot search results; isotopically labeled peptide belonging to NADPH oxidase is present only in the fraction collected from a signaling complex at the cell surface (labeled with the ICPL light +233.27) reagent and not control (expected label +239.22);

It may be possible to distinguish population of proteins from different receptor ligand or control beads by chemically modifying the different control or receptor complex proteins including modifications such as isotopic or isobaric labels labeling prior to mass spectrometry or prior to enzymatic digestion of modification proteins and mass spectrometry of the peptides. For example control beads incubated with crude homogenates showed no isotopically labeled NADPH oxidase while the samples from the IgG ligand coated bead showed the presence of isotopically labeled NADP oxidase, as protein know to associate with the Fc receptor.

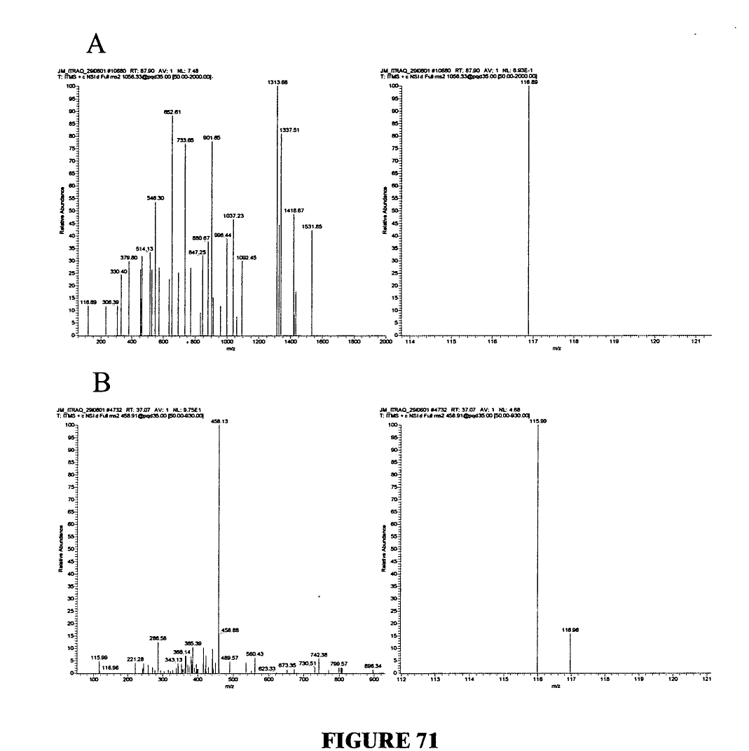

FIG. 71. ITRAQ isobarically labeled 116 control and 117 labeled IgG coated beads pulled from the cell membrane; A, Left panel shows MS/MS of protein PAK2 known and Right panel shows quantification, where it is only observed in the bead coated with IgG ligand when bound to cell surface and not in the control, B, Left panel shows MS/MS of RNA-binding region RNP-1 (RNA recognition motif), Right panel confirms that it is localized at 10× higher concentration in control non-specifically bound fraction than at the 117 labeled IgG ligand coated bead bound to the cell surface.

Proteins or peptides may be labeled with isotopic or isobaric or otherwise chemically modified to distinguish proteins associated with at least one receptor ligand coated bead compared to other receptor ligands or control beads. Peptides from digests of control beads were chemical modified to include an isobaric label resulting in a fragmentation product of 116 m/z while peptides from IgG coated beads were labeled with a chemical modification that resulted in the production of a 117 m/z product. The ratio of the chemical products can be used to differentiate between the control and IgG receptor associated proteins. For example PAK was associated with the IgG coated beads while a protein containing an RNA binding motif was associated with the control beads.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

1. A process for analyzing activated receptor signaling complexes from live cells comprising:
coating at least one bead with at least one receptor ligand which ligand binds to said live cells;
contacting said ligand coated bead with said live cells thereby forming at least one bead binding site; and
thereby initiating formation of at least one activated receptor signaling complex at said at least one bead binding site, which specifically and mutually binds said at least one bead via said ligand to at least one activated receptor;
whereby each said specifically activated receptor signaling complex may be isolated and subjected to biochemical or biophysical analysis.

2. A process for analyzing activated receptor signaling complexes from live cells comprising:
coating at least one bead with at least one receptor ligand which ligand binds to said live cells;
contacting said ligand coated bead with said live cells thereby forming at least one bead binding site; and
initiating formation of at least one activated receptor signaling complex at said at least one bead binding site, which specifically and mutually binds said at least one bead via said ligand to at least one activated receptor; and
whereby each said specifically activated receptor signaling complex may be isolated and subjected to biochemical or biophysical analysis in situ.

3. A process for capturing activated receptor signaling complexes from live cells comprising:
coating at least one bead with at least one receptor ligand which ligand binds to said live cells;
contacting said ligand coated bead with said live cells thereby forming at least one bead binding site; and
initiating formation of at least one activated receptor signaling complex at said at least one bead binding site, which specifically and mutually binds said at least one bead via said ligand to at least one activated receptor; and
disrupting or homogenizing said cells and collecting said activated receptor signaling complex;
whereby each said specifically activated receptor signaling complex may be isolated and subjected to biochemical or biophysical analysis.

4. A process for distinguishing non-specifically bound proteins from specifically bound activated receptor signaling complexes comprising:
providing at least one bead coated with at least one receptor ligand which ligand binds said bead to live cells, and initiates formation of at least one activated receptor signaling complex;
disrupting or homogenizing said live cells and collecting said at least one activated receptor signaling complex;
further providing at least one control bead;
forming a non-specifically bound control complex by disrupting said live cells and incubating a homogenate derived therefrom, or other non-specific mixture of proteins, in the presence of at least one control bead; and
distinguishing between said specifically bound receptor complexes and said non-specifically bound proteins.

5. A process in accordance with claim 4 wherein said step of distinguishing non-specifically bound proteins from specifically bound activated receptor signaling complexes is selected from the group consisting of database comparison, algorithmic subtractive analysis of ms or ms/ms spectra and differential chemical modifications including isotopic and isobaric tagging.

6. A process for identifying a cell biopolymer function modulating material comprising:
providing at least one bead coated with at least one receptor ligand which ligand binds to said live cells;
contacting said coated bead with said live cells thereby initiating formation of at least one activated receptor signaling complex, in conjunction with introduction of at least one amount of a putative cell biopolymer function modulating material on a surface or interior of a cell; and
determining effectiveness of each said at least one amount of said putative cell biopolymer receptor function modulating material to act as a modulator of said receptor biopolymer, by measuring receptor pathway function

7. Use of PI3K as a therapeutic target for modulating the engulfment or phagocytosis of modified particles;
wherein said modulation is effective to prevent macrophage foam cell precursors or foam cells from becoming filled with modified particles.

8. A process for modulating the engulfment or phagocytosis of modified particles by macrophage foam cell precursors and/or foam cells resultant therefrom comprising:
contacting said macrophage foam cell precursors or foam cells resultant therefrom with at least one therapeutic molecule targeted against PI3K Class 1 Alpha.

9. The process of claim 8 wherein said therapeutic molecule is silencing RNA specific to PI3K Class 1 Alpha.

10. The process of claim 8 wherein said therapeutic molecule is a compound effective to specifically inhibit enzymatic activity of PI3K.

11. Use of PAP-1 as a therapeutic target for modulating the engulfment or phagocytosis of modified particles;
wherein said modulation is effective to prevent macrophage foam cell precursors or foam cells from becoming filled with modified particles.

12. A process for modulating the engulfment or phagocytosis of modified particles by macrophage foam cell precursors and/or foam cells resultant therefrom comprising:
contacting said macrophage foam cell precursors or foam cells resultant therefrom with at least one therapeutic molecule targeted against PAP-1.

13. The process of claim 12 wherein said therapeutic molecule is a compound effective to specifically inhibit enzymatic activity of PAP-1.

14. Use of RhoG, RhoA or P115RhoGEF as a therapeutic target for modulating the engulfment or phagocytosis of modified particles;
wherein said modulation is effective to prevent macrophage foam cell precursors or foam cells from becoming filled with modified particles.

15. A process for modulating the engulfment or phagocytosis of modified particles by macrophage foam cell precursors and/or foam cells resultant therefrom comprising:
contacting said macrophage foam cell precursors or foam cells resultant therefrom with at least one therapeutic molecule targeted against RhoG, RhoA or P115RhoGEF therein.

16. The process of claim 15 wherein said therapeutic molecule is silencing RNA or a dominant negative mutant specific to RhoG, RhoA or P115RhoGEF.

17. Use of Crk1 or crk1 as a therapeutic target for modulating the engulfment or phagocytosis of modified particles;
wherein said modulation is effective to prevent macrophage foam cell precursors or foam cells from becoming filled with modified particles.

18. A process for modulating the engulfment or phagocytosis of modified particles by macrophage foam cell precursors and/or foam cells resultant therefrom comprising:
contacting said macrophage foam cell precursors or foam cells resultant therefrom with at least one therapeutic molecule targeted against Crk1 or crk1.

19. The process of claim 18 wherein said therapeutic molecule is silencing RNA specific to cCrk1 or crk1.

20. Use of at least one statin to prevent accumulation of particles in macrophage foam cell precursors.

21. A process in accordance with claim 6 wherein measuring of receptor pathway function is performed at said bead binding site by determining particle internalization, or by accumulation of proteins, or by changes in rate of production of metabolites or by ionic concentrations, or by a combination thereof.

22. A process for localizing the activation or inactivation of a signaling complex or enzyme in time and space on or within live cells comprising;
introduction of at least one fluorescent protein or fluorescent protein domain into or upon said live cells;
stimulating or activating said signaling complex pathway or enzyme therein by contact with at least one ligand coated bead; and
assaying said activation of said signaling complex pathway or enzyme therein by measuring an accumulation of fluorescent proteins or a change in the accumulation of fluorescent metabolite binding domains.

23. The process of claim 22, wherein said metabolite is a small molecule.

24. The process of claim 22, wherein said metabolite is a post translational modification of a protein.

25. The process of claim 22, wherein said post translational modification is phosphorylation.

26. A process for localizing the activation or inactivation of a signaling complex or enzyme in time and space on or within fixed cells comprising;
stimulating or activating said signaling complex pathway or enzyme therein by contact with at least one ligand coated bead; and
assaying said activation of said signaling complex pathway or enzyme therein by measuring an accumulation of a protein or a change in the accumulation of metabolite using a specific binding reagent.

27. A process for quantifying penetration, efficacy and specificity of a cell biopolymer function modulating material comprising:
introducing an amount of said function modulating material on a surface or interior of a cell, in conjunction with at least one fluorescent protein or fluorescent protein domain or a nucleic acid encoding said fluorescent protein or fluorescent protein domain in a manner effective to enable determining the penetration, efficacy and specificity of said cell biopolymer function modulating material;
stimulating or activating said signaling complex pathway or enzyme therein by contact with at least one ligand coated bead; and
assaying said activation of said signaling complex pathway or enzyme therein by measuring a change in the accumulation or mobility of fluorescent proteins or a change in the accumulation or mobility of fluorescent metabolite binding domains;
whereby said penetration, efficacy or specificity of said amount of protein modulating material is quantified.

28. Process for quantifying penetration, efficacy and specificity of a cell biopolymer function modulating material comprising:
introducing an amount of said function modulating material on a surface or interior of a cell,
stimulating or activating said signaling complex pathway or enzyme therein by contact with at least one ligand coated bead; and
assaying said activation of said signaling complex pathway or enzyme therein by measuring the engulfment or phagocytosis of modified particle;
whereby said penetration, efficacy or specificity of said amount of protein modulating material is quantified.

29. The process of claim 28, wherein said specific binding reagent is an antibody.

30. The method of claim 1 where the receptor ligand affixed to the beads is IgG or OX-LDL.

31. The method of claim 1 where the beads are isolated by homogenizing the cells in buffer or buffer with non-ionic detergents or nuclease enzymes and the beads purified by centrifugation through a dense medium.

32. The method of claim 31 wherein the dense medium is dissolved sucrose.

33. The method of claim 31 wherein the dense medium is osmotically active.

34. The method of claim 6 where the proteins are eluted from the isolated beads by salt solutions, chaeotropes, or mass spectrometry compatible detergents or acids or base.

35. The method of claim 34, further including a step of digestion of said eluted proteins with lytic enzymes

36. The method of claim 35 wherein said lytic enzymes are proteases.

37. The method of claim 6 where the proteins bound to beads are directly digested with lytic enzymes with or without the presence of organic solvents.

38. The method of claim 4 where proteins or peptides arising from said beads are identified.

39. The method of claim 38 where specific proteins and peptides identified by mass spectroscopy from beads coated with a receptor ligand that engaged a receptor on live cells and triggered assembly of a membrane receptor complex are differentiated from proteins in homogenates or growth media that non-specifically bind the control beads using computation.

40. The method of claim 39 utilizing BLAST searching for full length homology or for short nearly exact sequences, or database comparisons of exact peptide sequences, or unknown ions or ion fragments or isotopic and isobaric tags, to subtract non-specific proteins detected on the control beads and reveal a set of specifically associated proteins.

41. The method of claim 6 where cell biopolymers specifically associated with the receptor signaling complex assembled in response to engagement of the receptor ligand are confirmed to be bound to said ligand coated beads or at said receptor signaling complex of said cell at said binding site of said ligand coated bead using immunological techniques including at least one of western blots, immuno staining, ELISA, and cellular methods including expression of fluorescent proteins or binding of fluorescent antibodies.

42. The process for modulating the engulfment or phagocytosis of modified particles by macrophage foam cell precursors and/or foam cells resultant therefrom in accordance with any one of claims 8, 12, 15, or 18, wherein said foam cell precursors include leukocytes selected from RAW macrophages, J774 macrophages, U937 macrophages, human neutrophils or model cell expressing receptor complex proteins.

43. The process for identifying a cell biopolymer function modulating material in accordance with any one of claims 1, 2, 3, 4 or 6, wherein said receptor ligand is selected from the group consisting of Immunoglobulin G (IgG), lipopolysaccharides (LPS), oxidatively modified low density lipoprotein (OX-LDL), acetyl LDL, and cholesterol.

44. The method of claim 2 where the beads are intrinsically fluorescent, or are fluorescently labeled with at least one fluorescent molecule either directly or via the attachment of fluorescent antibodies and where the cell biopolymer function modulating agent may also be fluorescently labeled.

45. The method of claim 1 or 2 or 3 wherein said biophysical analysis includes at least one of microscopy using fluorescent normal, polarized, differential interference contrast (DIC) or fluorescent detection with microscopes, deconvolution microscopes, laser confocal microscopes and deconvolution laser microscopy.

46. The method of any one of claims 1, 2, 3, 4, 6, 8, 12, 15 or 18 where beads internalized or engulfed by macrophage are detected by staining all particles outside said cells using a first fluorescent dye and further detecting all the particles in intact or permeabilized cells using a second fluorescent dye such that when the emission of said external particles attributed to said first fluorescent dye is compared to the emission of said internal particles attributed to said second fluorescent dye, the total number and fluorescence associated with internalized particles and the ratio of emission intensities between said external and internal particles are readily calculated and integrated.

47. A process in accordance with any one of claims 1, 2, 3, 4, 6, 8, 12, 15 or 18 wherein internal particles are calculated by lysing the beads with water whereby said engulfed cells are directly imaged, wherein said engulfed cells are quantified using non-fluorescent imaging.

48. Use of the enzyme magnesium dependent PAP-1 as a drug target in phagocytic diseases such as atherosclerosis, cancer, arthritis, Alzheimer's and cancer and in processes that result in aging such as free radical production.

49. Use of the enzyme magnesium dependent PAP-1 as a drug target in inflammatory diseases such as cancer and arthritis.

50. Use of the enzyme magnesium dependent PAP-1 as a drug target in free radical diseases such as multiple sclerosis, ischemia, neurodegeneration or spinal cord injury.

51. Use of IgG, OX-LDL, Acetyl-LDL, or LD-LDL as a receptor ligand affixed onto beads for the purpose of discovering or validating drug targets or screening drugs in RAW 264.7 macrophages and other ligands in other cells.

52. A process for identifying a cell biopolymer function modulating material in RAW 264.7 macrophages comprising:
providing a coated bead by affixing to said bead at least one receptor ligand selected from the group consisting of IgG, OX-LDL, Acetyl-LDL, and LD-LDL;
contacting said RAW 264.7 macrophages with said coated bead to form at least one bead binding site, which specifically binds to said bead;
measuring receptor pathway function by determining particle internalization, by accumulation of proteins, or by changes in accumulation of metabolites, or ionic concentrations; and
determining effectiveness of said cell biopolymer function modulating material as a modulator of cell biopolymer function.

53. Use of at least one PROTEIN selected from the group consisting of P115, RhoG, RhoA, thrombospondin, PLC beta and PAP-1 as a therapeutic target for at least one phagocytic disease selected from atherosclerosis, arthritis, cancer, and Alzheimer's dementia.

54. Use of a RAW macrophage cell line to characterize and screen drugs and therapeutic agents against phagocytosis.

55. A process in accordance with claim 52 wherein said cell biopolymer function modulating material is linked to at least one phagocytic disease including atherosclerosis, arthritis, cancer and Alzheimer's dementia.

56. A process for confirming or establishing protein-protein or protein complex interactions with in situ measurements comprising:
coating at least one bead with at least one ligand which is capable of binding said bead to said live cells;
contacting said coated bead with said live cells thereby forming at least one bead binding site; and
initiating formation of a receptor complex at said at least one bead binding site, which specifically and mutually binds to said bead;
whereby rate of diffusion and bound fraction of said receptor complex biopolymers are measured directly by fluorescence recovery after photo-bleaching (FRAP) analysis.

57. Use of ligand coated beads to characterize kinetics of receptor or receptor pathway functions including modulation of cellular calcium, accumulation of biopolymers or metabolites, and changes in phosphorylations states of biopolymers.

58. Use of RAW macrophages as a model of foam cell formation in atherosclerotic disease to examine the effects of drugs on the expression, localization, phosphorylation or function of proteins in response to drug or stimulatory agents.
